(19) United States
(12) Patent Application Publication
(10) Pub. No.: US 2020/0316185 A1
(43) Pub. Date: Oct. 8, 2020

(54) VACCINES AND METHODS OF VACCINATION AGAINST SCHISTOSOMA

(71) Applicant: THE ROYAL INSTITUTION FOR THE ADVANCEMENT OF LEARNING/MCGILL UNIVERSITY, Montreal (CA)

(72) Inventors: Brian J. Ward, Montreal (CA); Adam Hassan, Montreal (CA); Momar Ndao, Montreal (CA)

(73) Assignee: THE ROYAL INSTITUTION FOR THE ADVANCEMENT OF LEARNING/MCGILL UNIVERSITY, Montreal (CA)

(21) Appl. No.: 16/812,212

(22) Filed: Mar. 6, 2020

Related U.S. Application Data
(60) Provisional application No. 62/816,029, filed on Mar. 8, 2019.
(60) Provisional application No. 62/895,492, filed on Sep. 3, 2019.
(60) Provisional application No. 62/860,556, filed on Jun. 12, 2019.

(51) Int. Cl. A61K 39/112; A61K 9/00; A61K 39/39; A61P 33/12

(52) U.S. Cl. CPC A61K 39/0275; A61K 9/0019; A61K 2039/54; A61P 33/12; A61K 39/39

(57) ABSTRACT

A method of immunizing a human against infection by parasitic worms, comprising orally administering a live attenuated recombinant bacterium, expressing at least one antigen corresponding to a parasitic worm antigen; and a sterile injectable vaccine comprising the at least one antigen corresponding to a parasitic worm antigen. The method is effective against worms, including schistosomes.

### CROSS REFERENCE TO RELATED APPLICATIONS

This application claims benefit of priority under 35 U.S.C. § 119(e), and is a non-provisional of, U.S. Provisional Patent Application No. 62/895,492, filed Sep. 3, 2019, and U.S. Provisional Patent Application 62/860,556, filed Jun. 12, 2019, and U.S. Provisional Patent Application 62/816,029, filed Mar. 8, 2019, each of which is expressly incorporated herein by reference in its entirety.

### FIELD OF THE INVENTION

The present invention relates to the field of vaccines and methods of vaccination against parasitic worms, and in particular use of vaccine components which employ a live bacterium to generate and deliver a Schistosoma antigen. See, Hassan, Adam S., Nicholas H. Zelt, Dilhan J. Perera, Momar Ndao, and Brian J. Ward. “Vaccination against the digestive enzyme Cathepsin B using a YS1646 Salmonella enterica Typhimurium vector provides almost complete protection against Schistosoma mansoni challenge in a mouse model.” bioRxiv (2019): 652644; Adam S Hassan, Nicholas H Zelt, Dilhan J Perera, Momar Ndao, Brian J Ward, “Vaccination against the digestive enzyme Cathepsin B using a YS1646 Salmonella enterica Typhimurium vector provides almost complete protection against Schistosoma mansoni challenge in a mouse model”, PLoS New Tropical Diseases (2019).

### BACKGROUND

All references cited herein are expressly incorporated by reference in their entirety.

Schistosomiasis is caused by a number of Schistosoma spp. These trematodes currently infect >250 million people worldwide and more than 800 million are at risk of infection [1]. The World Health Organization (WHO) considers schistosomiasis to be the most important human helminth infection in terms of mortality and morbidity [2]. Of the three main human schistosome species, S. mansoni is very widespread; causing a significant burden of disease in South America, Sub-Saharan Africa, and the Caribbean [3].

The current treatment of schistosomiasis relies heavily on the drug praziquantel (PZQ). This oral anthelminthic paralyzes the adult worms and has a reported efficacy of 85-90% [4]. The availability of only one effective drug is a precarious situation however and praziquantel resistance has been observed both experimentally [5, 6] and reduced PZQ cure rates have been observed in the field [7, 8]. Furthermore, praziquantel treatment does not prevent re-infection. There is a clear need for a vaccine that can be used in conjunction with mass drug administration (MDA) and vector control efforts.

The WHO Special Program for Research and Training in Tropical Diseases (TDR/WHO) has encouraged the search for a vaccine that can provide ≥40% protection against S. mansoni [9]. Despite this relatively ‘low bar’, few candidate vaccines have achieved >50% protection in murine or other animal models [10] and even fewer have progressed to human trials [11]. Our group has previously demonstrated 60-70% protection in a S. mansoni murine challenge model by targeting Cathepsin B using intramuscular (IM)-adjuvanted formulations [12, 13]. Cathepsin B (CatB) is a cysteine protease found in the cecum of both the migratory larval form of S. mansoni (ie: the schistosomula) and in the gut of the adult worm. CatB is important for the digestion of host blood macromolecules such as hemoglobin, serum albumin and immunoglobulin G (IgG) [14]. Suppression of CatB expression using RNA interference (RNAi) has a major impact on parasite growth and fitness [15]. Because the schistosomulae migrate through the lungs and the adult worms reside in mesenteric veins adjacent to the gut mucosa, we wished to determine if a vaccination strategy that targeted induction of both mucosal and systemic responses to CatB could improve protection.

YS1646 is a highly attenuated Salmonella enterica serovar Typhimurium carrying mutations in the msbB (lipopolysaccharide or LPS) and purI (purine biosynthesis pathway) genes that was originally developed as a possible cancer therapeutic [16]. Although its development was halted when it failed to provide benefit in a large phase I trial in subjects with advanced cancer, it was well-tolerated when administered intravenously at doses of up to 3.0×10⁸ colony-forming units/m² [16]. We are seeking to repurpose YS1646 as a novel vaccination platform and reasoned that a locally-invasive but highly attenuated Salmonella vector might induce both local and systemic responses to CatB. The flagellin protein of S. typhimurium has been proposed as a general mucosal adjuvant through its action on toll-like receptor (TLR) 5 [17]. Other Salmonella products such as LPS would be expected to further enhance immune responses by triggering TLR4 [18, 19]. Indeed, live attenuated Salmonella have multiple potential advantages as vaccine vectors and have been used to express foreign antigens against infectious diseases and cancers [20-22]. They directly target the intestinal microfold (M) cells overlying the gut-associated lymphoid tissues (GALT) [21, 23-26], have large ‘carrying’ capacity [27] and are easy to manipulate both in the laboratory and at industrial scale. Although there is considerable experience with the attenuated S. typhi vaccine strain (Ty21a: Vivotif™) in the delivery of heterologous antigens [21, 28], far less is known about the potential of other Salmonella strains. Of direct relevance to the current work, Chen and colleagues used YS1646 to express a chimeric S. japonicum antigen that induced both strong antibody and cellular responses after repeated oral dosing and provided up to 62% protection in a murine challenge model [29].

See, U.S. 20190017057; 20180271787; 20170157239; 20170051260; 20160222393; 20160028148; 20150017204; 20140220661; 20120142080; 20110223241; 20100136048; 20100135961; 20090169517; 20080124355; 20070009489; 20050255088; 20050249706; 20050052892; 20050036987; 20040219169; 20040042274; 20040037117; 20030170276; 20030113293; 20030109026; 20020026655; U.S. Pat. Nos. 10,286,051; 10,188,722; 10,141,626; 10,087,451; 9,878,023; 9,739,773; 9,737,592; 9,657,085; 9,616,114; 9,597,379; 9,593,339; 9,486,513; 9,421,252; 9,365,625; 9,315,817; 9,200,289; 9,200,251; 9,068,187; 8,956,859; 8,771,669; 8,647,642; 8,623,350; 8,524,220; 8,440,207; 8,241,623; 7,514,089; 7,452,531; 7,354,592; 7,211,843; 6,962,696; 6,934,176; 6,923,972; 6,863,894; 6,798,684; 6,685,935; 6,475,482; 6,447,784; 6,190,657; and 6,080,849.

In the mid-1990s, the Tropical Diseases Research (TDR) committee of the World Health Organization (WHO) launched the search for a S. mansoni vaccine candidate capable of providing ≥40% protection [9]. This initiative targeted reduced worm numbers as well as reductions in egg burden in both the liver and the intestinal tissues. S. mansoni female worms can produce hundreds of eggs per day [35]. While the majority are excreted in the feces, some are trapped in host tissues where they cause most of the pathology associated with chronic infection [36]. Eggs trapped in the liver typically induce a vigorous granulomatous response that can lead to fibrosis, portal hypertension and death while egg-induced granulomas in the intestine cause local lesions that contribute to colonic polyp formation [37]. Reducing the hepatic egg burden would therefore be predicted to decrease S. mansoni-associated morbidity and mortality while reducing the intestinal egg burden would likely decrease transmission.

The protective efficacy of CatB-based vaccines delivered IM with adjuvants has been previously described [12, 13]. Using CpG dinucleotides to promote a Th1-type response, vaccination resulted in a 59% reduction in worm burden after challenge with 56% and 54% decreases in hepatic and intestinal egg burden respectively compared to adjuvant-alone control animals [12]. Parasitologic outcomes were slightly better in the same challenge model when the oil-in-water adjuvant Montanide ISA 720 VG was used to improve the antibody response: 56-62% reductions in worm numbers and the egg burden in tissues [13]. These results were well above the 40% threshold suggested by the TDR/WHO and provided proof-of-concept for CatB as a promising target antigen. Based on this success, we expanded our vaccine discovery program to explore alternate strategies and potentially more powerful delivery systems. The availability of the highly attenuated Salmonella enterica Typhimurium strain YS1646 that had been used in a phase 1 clinical cancer trial at doses up to 3×10⁸ IV was attractive for many reasons. Although S. enterica species replicate in a membrane-bound host cell compartment or vacuole [38], foreign protein antigens can be efficiently exported from the vacuole into the cytoplasm using the organism's T3SS. Like all Salmonella enterica species, YS1646 has two distinct T3SS located in Salmonella pathogenicity islands 1 and 2 (SPI-I and SPI-II) [39] that are active at different phases of infection [40]. The SPI-I T3SS translocates proteins upon first contact of the bacterium with epithelium cells through to the stage of early cell invasion while SPI-II expression is induced once the bacterium has been phagocytosed [41]. These T3SS have been used by many groups to deliver heterologous antigens in Salmonella-based vaccine development programs [22, 42, reviewed by Galen J E, Buskirk A D, Tennant S M, Pasetti M F, “Live Attenuated Human Salmonella Vaccine Candidates: Tracking the Pathogen in Natural Infection and Stimulation of Host Immunity”, EcoSal Plus. 2016 November; 7(1). doi: 10.1128/ecosalplus.ESP-0010-2016].

In recent years, live attenuated Salmonella has been increasingly used to express foreign antigens against infectious diseases and cancers. [Clark-Curtiss J E, Curtiss R. 2018. Salmonella Vaccines: Conduits for Protective Antigens. Journal of immunology (Baltimore, Md.: 1950) 200:39-48; Galen J E, Buskirk A D, Tennant S M, Pasetti M F. 2016. Live Attenuated Human Salmonella Vaccine Candidates: Tracking the Pathogen in Natural Infection and Stimulation of Host Immunity. EcoSal Plus 7; Panthel K, Meinel K M, Sevil Domènech V E E, Trülzsch K, Rüssmann H. 2008. Salmonella type III-mediated heterologous antigen delivery: a versatile oral vaccination strategy to induce cellular immunity against infectious agents and tumors. International journal of medical microbiology: IJMM 298:99-103.; Bolhassani, Azam, and Farnaz Zahedifard. “Therapeutic live vaccines as a potential anticancer strategy.” International journal of cancer 131, no. 8 (2012): 1733-1743; Medina, Eva, and Carlos Alberto Guzmán. “Use of live bacterial vaccine vectors for antigen delivery: potential and limitations.” Vaccine 19, no. 13-14 (2001): 1573-1580; Seegers, Jos F M L. “Lactobacilli as live vaccine delivery vectors: progress and prospects.” Trends in biotechnology 20, no. 12 (2002): 508-515; Shams, Homayoun. “Recent developments in veterinary vaccinology.” The veterinary journal 170, no. 3 (2005): 289-299; Kang, Ho Young, Jay Srinivasan, and Roy Curtiss. “Immune responses to recombinant pneumococcal PspA antigen delivered by live attenuated Salmonella enterica serovar Typhimurium vaccine.” Infection and immunity 70, no. 4 (2002): 1739-1749; Cardenas, Lucia, and J. D. Clements. “Oral immunization using live attenuated Salmonella spp. as carriers of foreign antigens.” Clinical microbiology reviews 5, no. 3 (1992): 328-342; Buckley, Anthony M., Jinhong Wang, Debra L. Hudson, Andrew J. Grant, Michael A. Jones, Duncan J. Maskell, and Mark P. Stevens. “Evaluation of live-attenuated Salmonella vaccines expressing Campylobacter antigens for control of C. jejuni in poultry.” Vaccine 28, no. 4 (2010): 1094-1105; Dougan, G., C. E. Hormaeche, and D. J. Maskell. “Live oral Salmonella vaccines: potential use of attenuated strains as carriers of heterologous antigens to the immune system.” Parasite immunology 9, no. 2 (1987): 151-160; Mastroeni, Pietro, Bernardo Villarreal-Ramos, and Carlos E. Hormaeche. “Role of T cells, TNFα and IFNγ in recall of immunity to oral challenge with virulent Salmonellae in mice vaccinated with live attenuated aro− Salmonella vaccines.” Microbial pathogenesis 13, no. 6 (1992): 477-491; Galen, James E., Oscar G. Gomez-Duarte, Genevieve A. Losonsky, Jane L. Halpern, Carol S. Lauderbaugh, Shevon Kaintuck, Mardi K. Reymann, and Myron M. Levine. “A murine model of intranasal immunization to assess the immunogenicity of attenuated Salmonella typhi live vector vaccines in stimulating serum antibody responses to expressed foreign antigens.” Vaccine 15, no. 6-7 (1997): 700-708; Shahabi, Vafa, Paulo C. Maciag, Sandra Rivera, and Anu Wallecha. “Live, attenuated strains of Listeria and Salmonella as vaccine vectors in cancer treatment.” Bioengineered bugs 1, no. 4 (2010): 237-245; Fraillery, Dominique, David Baud, Susana Yuk-Ying Pang, John Schiller, Martine Bobst, Nathalie Zosso, Françoise Ponci, and Denise Nardelli-Haefliger. “Salmonella enterica serovar Typhi Ty21a expressing human papillomavirus type 16 L1 as a potential live vaccine against cervical cancer and typhoid fever.” Clin. Vaccine Immunol. 14, no. 10 (2007): 1285-1295; Paterson, Yvonne, Patrick D. Guirnalda, and Laurence M. Wood. “Listeria and Salmonella bacterial vectors of tumor-associated antigens for cancer immunotherapy.” In Seminars in immunology, vol. 22, no. 3, pp. 183-189. Academic Press, 2010; Wieckowski, Sébastien, Lilli Podola, Marco Springer, Iris Kobl, Zina Koob, Caroline Mignard, Amine Adda Berkane et al. “Immunogenicity and antitumor efficacy of live attenuated Salmonella typhimurium-based oral T-cell vaccines VXM01m, VXM04m and VXM06m.” (2017): 4558-4558; Wieckowski, Sébastien, Lilli Podola, Marco Springer, Iris Kobl, Zina Koob, Caroline Mignard, Alan Broadmeadow et al. “Non-clinical safety, immunogenicity and antitumor efficacy of live attenuated Salmonella typhimurium-based oral T-cell vaccines VXM01m, VXM04m and VXM06m.” In Molecular Therapy, vol. 25, no. 5, pp. 360-360. 50 Hampshire St, Floor 5, Cambridge, Mass. 02139 USA: Cell Press, 2017; Wieckowski, Sébastien, Lilli Podola, Heiko Smetak, Anne-Lucie Nugues, Philippe Slos, Amine Adda Berkane, Ming Wei et al. “Modulating T cell immunity in tumors by targeting PD-L1 and neoantigens using a live attenuated oral Salmonella platform.” (2018): 733-733; Vendrell, Alejandrina, Claudia Mongini, Maria José Gravisaco, Andrea Canellada, Agustina Inés Tesone, Juan Carlos Goin, and Claudia Inés Waldner. “An oral Salmonella-based vaccine inhibits liver metastases by promoting tumor-specific T-cell-mediated immunity in celiac and portal lymph nodes: a preclinical study.” Frontiers in Immunology 7 (2016): 72.)

Salmonella enterica is a facultative intracellular pathogen that replicates in a unique membrane-bound host cell compartment, the Salmonella-containing vacuole [Ibarra J A, Steele-Mortimer O. 2009. Salmonella—the ultimate insider. Salmonella virulence factors that modulate intracellular survival. Cell Microbiol 11:1579-1586.]. Although this location limits exposure of both Salmonella and foreign proteins produced by the bacterium to the immune system, the organism's type III secretion systems (T3SS) can be exploited to translocate heterologous antigens into the host cell cytoplasm. Salmonella enterica encodes two distinct T3SS within the Salmonella pathogenicity islands 1 and 2 (SPI-I and SPI-II) that become active at different phases of infection [Gerlach R G, Hensel M. 2007. Salmonella pathogenicity islands in host specificity, host pathogen-interactions and antibiotics resistance of Salmonella enterica. Berl Munch Tierarztl Wochenschr 120:317-327.]. The SPI-I T3SS translocates effector proteins upon first contact of the bacterium with epithelium cells through to the stage of early cell invasion. In contrast, SPI-II expression is induced when the bacterium has been phagocytosed. Several effector proteins translocated by these T3SSs have been tested in the promotion of heterologous antigen expression in Salmonella-based vaccine development programs but how effector protein-mediated secretion of heterologous antigens affects immune responses is still poorly understood. [Panthel K, Meinel K M, Sevil Domènech V E E, Trülzsch K, Rüssmann H. 2008. Salmonella type III-mediated heterologous antigen delivery: a versatile oral vaccination strategy to induce cellular immunity against infectious agents and tumors. International journal of medical microbiology: IJMM 298:99-103; Xiong G, Husseiny M I, Song L, Erdreich-Epstein A, Shackleford G M, Seeger R C, Jackel D, Hensel M, Metelitsa L S. 2010. Novel cancer vaccine based on genes of Salmonella pathogenicity island 2. Int J Cancer 126:2622-2634.]

There is considerable experience in using the attenuated S. typhi vaccine strain (Ty21a: Vivotif™) in the delivery of heterologous antigens [Panthel K, Meinel K M, Sevil Domènech V E E, Trülzsch K, Rüssmann H. 2008. Salmonella type III-mediated heterologous antigen delivery: a versatile oral vaccination strategy to induce cellular immunity against infectious agents and tumors. International journal of medical microbiology: IJMM 298:99-103.]. However, S. typhimurium YS1646 was selected as a candidate vector. This strain is attenuated by mutations in its msbB (LPS) and purI (purine biosynthesis pathway) genes and was originally developed as a non-specific ‘cancer vaccine’ for solid tumors. With a major investment from Vion Inc., YS1646 was carried through pre-clinical and toxicity testing in rodents, dogs and non-human primates before a phase I clinical trial where it ultimately failed [Clairmont C, Lee K C, Pike J, Ittensohn M, Low K B, Pawelek J, Bermudes D, Brecher S M, Margitich D, Turnier J, Li Z, Luo X, King I, Zheng L M. 2000. Biodistribution and genetic stability of the novel antitumor agent VNP20009, a genetically modified strain of Salmonella typhimurium. The Journal of infectious diseases 181:1996-2002.]. More recently, YS1646 has been used to express a chimeric Schistosoma japonicum antigen that was tested in a murine model of schistosomiasis [Toso J F, Gill V J, Hwu P, Marincola F M, Restifo N P, Schwartzentruber D J, Sherry R M, Topalian S L, Yang J C, Stock F, Freezer L J, Morton K E, Seipp C, Haworth L, Mavroukakis S, White D, MacDonald S, Mao J, Sznol M, Rosenberg S A. 2002. Phase I study of the intravenous administration of attenuated Salmonella typhimurium to patients with metastatic melanoma. Journal of clinical oncology: official journal of the American Society of Clinical Oncology 20:142-152.]. Repeated oral administration of one of the engineered strains in this study elicited a strong systemic IgG antibody response, induced antigen-specific T cells and provided up to 75% protection against S. japonicum challenge.

The present technology, according to various embodiments, consists of known and/or antigens, chimeric proteins, or combinations of proteins, that are expressed, secreted, surface displayed and/or released by bacteria and result in immunologic activity, and may optionally include the combination with secreted protease inhibitors. The bacterial delivery vector may be attenuated, non-pathogenic, low pathogenic (including wild type), or a probiotic bacterium. The bacteria are introduced either systemically (e.g., parenteral, intravenous (IV), intramuscular (IM), intralymphatic (IL), intradermal (ID), subcutaneously (sub-q), local-regionally (e.g., intralesionally, intratumorally (IT), intraperitoneally (IP), topically, intrathecally (intrathecal), by inhaler or nasal spray) or to the mucosal system through oral, nasal, pulmonary intravessically, enema or suppository administration where they are able to undergo limited replication, express, surface display, secrete and/or release the anti-cancer inhibitory proteins or a combination thereof, and thereby provide a therapeutic or preventive benefit.

Promoters, i.e., genetic regulatory elements that control the expression of the genes encoding the therapeutic molecules described above that are useful in the present technology, according to various embodiments, include constitutive and inducible promoters. A preferred constitutive promoter is that from the vector pTrc99a (Promega). Preferred inducible promoters include the tetracycline inducible promoter (TET promoter), colicin promoters, sulA promoters and hypoxic-inducible promoters including but not limited to the PepT promoter (Bermudes et al., WO 01/25397), the arabinose inducible promoter (AraBAD) (Lossner et al., 2007, Cell Microbiol. 9: 1529-1537; WO/2006/048344) the salicylate (aspirin) derivatives inducible promoter (Royo et al., 2007, Nature Methods 4: 937-942; WO/2005/054477), or a quorum-sensing (autoinduction) promoter Anerson et al., 2006 Environmentally controlled invasion of cancer cells by engineered bacteria, J. Mol. Biol. 355: 619-627.

A single promoter may be used to drive the expression of more than one gene, such as an antigen and a protease inhibitor. The genes may be part of a single synthetic operon (polycistronic), or may be separate, monocystronic constructs, with separate individual promoters of the same type used to drive the expression of their respective genes. The promoters may also be of different types, with different genes expressed by different constitutive or inducible promoters. Use of two separate inducible promoters for more than one antigen or other effector type peptide allows, when sufficient tetracycline, arabinose or salicylic acid is administered following administration of the bacterial vector, their expression to occur simultaneously, sequentially, or alternatingly (i.e., repeated). An inducible promoter is not required, and a constitutive promoter may be employed.

The present technology, according to various embodiments, consists of known and/or antigens, chimeric proteins, or combinations of proteins, that are expressed, secreted, surface displayed and/or released by bacteria and result in immunologic activity, and may optionally include the combination with secreted protease inhibitors. The bacterial delivery vector may be attenuated, non-pathogenic, low pathogenic (including wild type), or a probiotic bacterium. The bacteria are introduced either systemically (e.g., parenteral, intravenous (IV), intramuscular (IM), intralymphatic (IL), intradermal (ID), subcutaneously (sub-q), local-regionally (e.g., intralesionally, intratumorally (IT), intraperitoneally (IP), topically, intrathecally (intrathecal), by inhaler or nasal spray) or to the mucosal system through oral, nasal, pulmonary intravessically, enema or suppository administration where they are able to undergo limited replication, express, surface display, secrete and/or release the anti-cancer inhibitory proteins or a combination thereof, and thereby provide a therapeutic or preventive benefit.

1. Weerakoon K G A D, Gobert G N, Cai P, McManus D P. Advances in the Diagnosis of Human Schistosomiasis. Clinical Microbiology Reviews. 2015; 28(4):939-67.

2. King C L. Chapter 68—Schistosomiasis. In: Barrett A D T, Stanberry L R, editors. Vaccines for Biodefense and Emerging and Neglected Diseases. London: Academic Press; 2009. p. 1401-21.

3. McManus D P, Dunne D W, Sacko M, Utzinger J, Vennervald B J, Zhou X-N. Schistosomiasis. Nature Reviews Disease Primers. 2018; 4(1):13.

4. Cioli D, Pica-Mattoccia L, Basso A, Guidi A. Schistosomiasis control: praziquantel forever? Molecular and biochemical parasitology. 2014; 195(1):23-9.

5. Couto F F, Coelho P M, Araujo N, Kusel J R, Katz N, Jannotti-Passos L K, et al. Schistosoma mansoni: a method for inducing resistance to praziquantel using infected Biomphalaria glabrata snails. Memorias do Instituto Oswaldo Cruz. 2011; 106(2):153-7.

6. Ismail M M, Farghaly A M, Dyab A K, Afify H A, el-Shafei M A. Resistance to praziquantel, effect of drug pressure and stability test. Journal of the Egyptian Society of Parasitology. 2002; 32(2):589-600.

7. Fenwick A, Webster J P. Schistosomiasis: challenges for control, treatment and drug resistance. Current opinion in infectious diseases. 2006; 19(6):577-82.

8. Melman S D, Steinauer M L, Cunningham C, Kubatko L S, Mwangi I N, Wynn N B, et al. Reduced susceptibility to praziquantel among naturally occurring Kenyan isolates of Schistosoma mansoni. PLoS neglected tropical diseases. 2009; 3(8):e504.

9. McManus D P, Loukas A. Current status of vaccines for schistosomiasis. Clin Microbiol Rev. 2008; 21(1):225-42.

10. Tebeje B M, Harvie M, You H, Loukas A, McManus D P. Schistosomiasis vaccines: where do we stand? Parasites & vectors. 2016; 9(1):528.

11. Merrifield M, Hotez P J, Beaumier C M, Gillespie P, Strych U, Hayward T, et al. Advancing a vaccine to prevent human schistosomiasis. Vaccine. 2016; 34(26):2988-91.

12. Ricciardi A, Dalton J P, Ndao M. Evaluation of the immune response and protective efficacy of Schistosoma mansoni Cathepsin B in mice using CpG dinucleotides as adjuvant. Vaccine. 2015; 33(2):346-53.

13. Ricciardi A, Visitsunthorn K, Dalton J P, Ndao M. A vaccine consisting of Schistosoma mansoni cathepsin B formulated in Montanide ISA 720 VG induces high level protection against murine schistosomiasis. BMC infectious diseases. 2016; 16:112.

14. Sajid M, McKerrow J H, Hansell E, Mathieu M A, Lucas K D, Hsieh I, et al. Functional expression and characterization of Schistosoma mansoni cathepsin B and its trans-activation by an endogenous asparaginyl endopeptidase. Molecular and biochemical parasitology. 2003; 131(1):65-75.

15. Correnti J M, Brindley P J, Pearce E J. Long-term suppression of cathepsin B levels by RNA interference retards schistosome growth. Molecular and biochemical parasitology. 2005; 143(2):209-15.

16. Toso J F, Gill V J, Hwu P, Marincola F M, Restifo N P, Schwartzentruber D J, et al. Phase I study of the intravenous administration of attenuated Salmonella typhimurium to patients with metastatic melanoma. Journal of clinical oncology: official journal of the American Society of Clinical Oncology. 2002; 20(1):142-52.

17. Makvandi M, Teimoori A, Parsa Nahad M, Khodadadi A, Cheshmeh M G D, Zandi M. Expression of Salmonella typhimurium and Escherichia coli flagellin protein and its functional characterization as an adjuvant. Microbial pathogenesis. 2018; 118:87-90.

18. Hayashi F, Smith K D, Ozinsky A, Hawn T R, Yi E C, Goodlett D R, et al. The innate immune response to bacterial flagellin is mediated by Toll-like receptor 5. Nature. 2001; 410(6832):1099-103.

19. Poltorak A, He X, Smirnova I, Liu M Y, Van Huffel C, Du X, et al. Defective LPS signaling in C3H/HeJ and C57BL/10ScCr mice: mutations in Tlr4 gene. Science (New York, N.Y.). 1998; 282(5396):2085-8.

20. Clark-Curtiss J E, Curtiss R, 3rd. Salmonella Vaccines: Conduits for Protective Antigens. Journal of immunology (Baltimore, Md.: 1950). 2018; 200(1):39-48.

21. Galen J E, Buskirk A D, Tennant S M, Pasetti M F. Live Attenuated Human Salmonella Vaccine Candidates: Tracking the Pathogen in Natural Infection and Stimulation of Host Immunity. EcoSal Plus. 2016; 7(1).

22. Panthel K, Meinel K M, Sevil Domenech V E, Trulzsch K, Russmann H. Salmonella type III-mediated heterologous antigen delivery: a versatile oral vaccination strategy to induce cellular immunity against infectious agents and tumors. International journal of medical microbiology: IJMM. 2008; 298(1-2):99-103.

23. Jepson M A, Clark M A. The role of M cells in Salmonella infection. Microbes and infection. 2001; 3(14-15):1183-90.

24. Hohmann E L, Oletta C A, Loomis W P, Miller S I. Macrophage-inducible expression of a model antigen in Salmonella typhimurium enhances immunogenicity. Proceedings of the National Academy of Sciences of the United States of America. 1995; 92(7):2904-8.

25. Penha Filho R A, Moura B S, de Almeida A M, Montassier H J, Barrow P A, Berchieri Junior A. Humoral and cellular immune response generated by different vaccine programs before and after Salmonella Enteritidis challenge in chickens. Vaccine. 2012; 30(52):7637-43.

26. Sztein M B. Cell-mediated immunity and antibody responses elicited by attenuated Salmonella enterica Serovar Typhi strains used as live oral vaccines in humans. Clinical infectious diseases: an official publication of the Infectious Diseases Society of America. 2007; 45 Suppl 1:S15-9.

27. Miller S I, Pulkkinen W S, Selsted M E, Mekalanos J J. Characterization of defensin resistance phenotypes associated with mutations in the phoP virulence regulon of Salmonella typhimurium. Infection and immunity. 1990; 58(11):3706-10.

28. Gentschev I, Spreng S, Sieber H, Ures J, Mollet F, Collioud A, et al. Vivotif—a ‘magic shield’ for protection against typhoid fever and delivery of heterologous antigens. Chemotherapy. 2007; 53(3):177-80.

29. Chen G, Dai Y, Chen J, Wang X, Tang B, Zhu Y, et al. Oral delivery of the Sj23LHD-GST antigen by Salmonella typhimurium type III secretion system protects against Schistosoma japonicum infection in mice. PLoS neglected tropical diseases. 2011; 5(9):e1313.

30. Yam K K, Gupta J, Winter K, Allen E, Brewer A, Beaulieu E, et al. AS03-Adjuvanted, Very-Low-Dose Influenza Vaccines Induce Distinctive Immune Responses Compared to Unadjuvanted High-Dose Vaccines in BALB/c Mice. Frontiers in immunology. 2015; 6:207.

31. Frey A, Di Canzio J, Zurakowski D. A statistically defined endpoint titer determination method for immunoassays. Journal of immunological methods. 1998; 221(1-2):35-41.

32. Tucker M S, Karunaratne L B, Lewis F A, Freitas T C, Liang Y S. Schistosomiasis. Current protocols in immunology. 2013; 103:Unit 19.1.

33. Cronan M R, Matty M A, Rosenberg A F, Blanc L, Pyle C J, Espenschied S T, et al. An explant technique for high-resolution imaging and manipulation of mycobacterial granulomas. Nature Methods. 2018; 15(12):1098-107.

34. Ebenezer J A, Christensen J M, Oliver B G, Oliver R A, Tjin G, Ho J, et al. Periostin as a marker of mucosal remodelling in chronic rhinosinusitis. Rhinology. 2017; 55(3):234-41.

35. Loverde P T, Chen L. Schistosome female reproductive development. Parasitology today (Personal ed). 1991; 7(11):303-8.

36. Elbaz T, Esmat G. Hepatic and intestinal schistosomiasis: review. Journal of advanced research. 2013; 4(5):445-52.

37. Mohamed A R, al Karawi M, Yasawy M I. Schistosomal colonic disease. Gut. 1990; 31(4):439-42.

38. Ibarra J A, Steele-Mortimer O. Salmonella—the ultimate insider. Salmonella virulence factors that modulate intracellular survival. Cellular microbiology. 2009; 11(11):1579-86.

39. Haraga A, Ohlson M B, Miller S I. Salmonellae interplay with host cells. Nature reviews Microbiology. 2008; 6(1):53-66.

40. Gerlach R G, Hensel M. Salmonella pathogenicity islands in host specificity, host pathogen-interactions and antibiotics resistance of Salmonella enterica. Berliner und Munchener tierarztliche Wochenschrift. 2007; 120(7-8):317-27.

41. Lee A K, Detweiler C S, Falkow S. OmpR regulates the two-component system SsrA-ssrB in Salmonella pathogenicity island 2. Journal of bacteriology. 2000; 182(3):771-81.

42. Xiong G, Husseiny M I, Song L, Erdreich-Epstein A, Shackleford G M, Seeger R C, et al. Novel cancer vaccine based on genes of Salmonella pathogenicity island 2. International journal of cancer. 2010; 126(11):2622-34.

43. Beaumier C M, Gillespie P M, Hotez P J, Bottazzi M E. New vaccines for neglected parasitic diseases and dengue. Translational research: the journal of laboratory and clinical medicine. 2013; 162(3):144-55.

44. Tendler M, Simpson A J. The biotechnology-value chain: development of Sm14 as a schistosomiasis vaccine. Acta tropica. 2008; 108(2-3):263-6.

45. Pearson M S, Pickering D A, McSorley H J, Bethony J M, Tribolet L, Dougall A M, et al. Enhanced protective efficacy of a chimeric form of the schistosomiasis vaccine antigen Sm-TSP-2. PLoS neglected tropical diseases. 2012; 6(3):e1564.

46. Tran M H, Pearson M S, Bethony J M, Smyth D J, Jones M K, Duke M, et al. Tetraspanins on the surface of Schistosoma mansoni are protective antigens against schistosomiasis. Nature medicine. 2006; 12(7):835-40.

47. Ahmad G, Torben W, Zhang W, Wyatt M, Siddiqui A A. Sm-p80-based DNA vaccine formulation induces potent protective immunity against Schistosoma mansoni. Parasite immunology. 2009; 31(3):156-61.

48. Su F, Patel G B, Hu S, Chen W. Induction of mucosal immunity through systemic immunization: Phantom or reality? Human vaccines & immunotherapeutics. 2016; 12(4):1070-9.

49. Wahid R, Pasetti M F, Maciel M, Jr., Simon J K, Tacket C O, Levine M M, et al. Oral priming with Salmonella typhi vaccine strain CVD 909 followed by parenteral boost with the S. typhi Vi capsular polysaccharide vaccine induces CD27+IgD-S. Typhi-specific IgA and IgG B memory cells in humans. Clinical immunology (Orlando, Fla.). 2011; 138(2):187-200.

50. Grzych J M, Grezel D, Xu C B, Neyrinck J L, Capron M, Ouma J H, et al. IgA antibodies to a protective antigen in human Schistosomiasis mansoni. Journal of immunology (Baltimore, Md.: 1950). 1993; 150(2):527-35.

51. Mangold B L, Dean D A. Passive transfer with serum and IgG antibodies of irradiated cercaria-induced resistance against Schistosoma mansoni in mice. Journal of immunology (Baltimore, Md.: 1950). 1986; 136(7):2644-8.

52. Melo T T, Sena I C, Araujo N, Fonseca C T. Antibodies are involved in the protective immunity induced in mice by Schistosoma mansoni schistosomula tegument (Smteg) immunization. Parasite immunology. 2014; 36(2):107-11.

53. Cuburu N, Kim R, Guittard G C, Thompson C D, Day P M, Hamm D E, et al. A Prime-Pull-Amplify Vaccination Strategy To Maximize Induction of Circulating and Genital-Resident Intraepithelial CD8(+) Memory T Cells. Journal of immunology (Baltimore, Md.: 1950). 2019; 202(4):1250-64.

54. Pearce E J, C M K, Sun J, J J T, McKee A S, Cervi L. Th2 response polarization during infection with the helminth parasite Schistosoma mansoni. Immunological reviews. 2004; 201:117-26.

55. El Ridi R, Tallima H, Selim S, Donnelly S, Cotton S, Gonzales Santana B, et al. Cysteine peptidases as schistosomiasis vaccines with inbuilt adjuvanticity. PloS one. 2014; 9(1):e85401.

56. Ricciardi A, Zelt N H, Visitsunthorn K, Dalton J P, Ndao M. Immune Mechanisms Involved in Schistosoma mansoni-Cathepsin B Vaccine Induced Protection in Mice. Frontiers in immunology. 2018; 9:1710.

57. Wang J Y, Harley R H, Galen J E. Novel methods for expression of foreign antigens in live vector vaccines. Human vaccines & immunotherapeutics. 2013; 9(7):1558-64.

58. Glenting J, Wessels S. Ensuring safety of DNA vaccines. Microbial cell factories. 2005; 4:26.

59. Hindle Z, Chatfield S N, Phillimore J, Bentley M, Johnson J, Cosgrove C A, et al. Characterization of Salmonella enterica derivatives harboring defined aroC and Salmonella pathogenicity island 2 type III secretion system (ssaV) mutations by immunization of healthy volunteers. Infection and immunity. 2002; 70(7):3457-67.

60. Gryseels B, Polman K, Clerinx J, Kestens L. Human schistosomiasis. Lancet (London, England). 2006; 368(9541):1106-18.

61. Ferrari M L, Coelho P M, Antunes C M, Tavares C A, da Cunha A S. Efficacy of oxamniquine and praziquantel in the treatment of Schistosoma mansoni infection: a controlled trial. Bulletin of the World Health Organization. 2003; 81(3):190-6.

62. Loverde P T, Chen L. Schistosome female reproductive development. Parasitology today (Personal ed). 1991; 7(11):303-8.

63. Mountford, A. P., A. Fisher, and R. A. Wilson, The profile of IgG1 and IgG2a antibody responses in mice exposed to Schistosoma mansoni. Parasite Immunol, 1994. 16(10): p. 521-7.

The infective cycle of Schistosoma mansoni involves asexual reproduction within an intermediate snail host, followed by infection of a human host. Cercariae, the larval stage which exits from an intermediate snail host, infect humans by penetrating human skin. These juvenile schistosomes mature to schistosomula, undergo an intricate migration through the host's lungs and liver, and develop into sexually mature egg-laying adults. Sexually mature male and female schistosomes begin the egg-laying phase of the life cycle within the intestinal venules. The constant production of large numbers of ova results in the excretion of some eggs with fecal matter, and in heavy infection, entrapment of eggs in visceral organs with ensuing host granulomatous immune responses directed against them. It is this egg-induced organ damage which results in complications such as hepatic fibrosis, portal hypertension, and esophageal varices, which lead to the death of chronically infected hosts.

The chronic nature of this debilitating disease results in cumulative damage to the liver, spleen, and colon due to the granulomatous reaction to accumulated embryonated eggs. Infection results in the production of circulating anti-schistosomal antibodies. The immune response is erratic, however, and does not lead to sterile immunity. Additionally, the adult parasites evade immune clearance by complex and multifactorial mechanisms.

Several adult S. mansoni proteins have been considered as potential vaccine candidates. Ideally, the most promising vaccine candidates may be those which are surface-exposed and are indispensable for the parasite's survival within the human host.

Schistosomes interact closely with their host, performing functions such as immune evasion, nutrient uptake, and attachment. Host-exposed schistosome proteins that undertake such essential functions are effective targets for a schistosomiasis vaccine. One such protein is the large subunit of calpain (Sm-p80) which plays an important role in the surface membrane renewal of schistosomes, an immune evasion mechanism employed by blood-dwelling helminths to evade host immunity. Sm-p80 is exposed at the host parasite interface and is naturally immunogenic. While the natural immunogenicity of the molecule does not provide protection under conditions of natural infection, it is possible to present calpain to the immune system in such a way as to induce potent immunity. The UNDP/World Bank/WHO-TDR special panel designated Sm-p80 as one of the priority antigens “with established credentials, needing further development” and Sm-p80 is now considered as one of the “first-tier candidates” by international experts in the field.

The T3SS secretion system is discussed in U.S. 2019/0055569, 2010/0120124, 2012/0021517, 2015/0359909, U.S. Pat. Nos. 9,951,340, 6,306,387, expressly incorporated herein by reference.

Some bacterial pathogens comprise a type three secretion system (T3SS), which serves as a needle-like system for delivering bacterial polypeptides (effectors) into host cells. These effector polypeptides typically contribute to the virulence of the bacterial cell. In contrast, commensal microbes have not been described to comprise a T3SS.

A T3SS is a multi-protein structure found in gram negative bacteria. It moves polypeptides from the cytoplasm of the bacterial cell through the interior of the T3SS “needle” into the cytoplasm of a target cell. T3SS's are found in pathogenic strains and have been observed in pathogenic isolates of, e.g., Shigella, Salmonella, E. coli, Burkholderia, Yersinia, Chlamydia, Pseudomonas, Erwinia, Ralstonia, Rhizobium, Vibrio, and Xanthamonas. Further discussion of T3SS's can be found, e.g. in Izore et al. Structure 2011 19:603-612; Korotkov et al. Nature Reviews Microbiology 2012 10:336-351; Wooldridge, K. (ed) Bacterial Secreted Proteins. Caster Academic Press 2009; Snyder and Champness (eds.) Molecular Genetics of Bacteria. 3rd Ed. ASM Press: 2007; each of which is incorporated by reference herein in its entirety.

The suite of T3SS-related proteins in a given wild-type cell is typically divided into structural proteins (those proteins which form the needle itself), substrate proteins (those proteins which are transported through the needle to the host), and chaperones (those proteins that bind effectors in the cytoplasm to protect, process, and/or shuttle the effectors to the needle). As used herein, a “functional T3SS” refers, minimally, to the set of structural proteins which are required in order to transfer at least one polypeptide to a target cell. In some embodiments, a functional T3SS system can comprise one or more chaperone proteins. In some embodiments, a functional T3SS can comprise one or more, for example, two, three, or four, substrates which are not virulence factor (e.g. certain translocators). In some embodiments, a functional T3SS does not comprise a virulence factor which is delivered to the target cell.

As used herein, a “virulence factor” refers to those substrates which affect and/or manipulate a target cell in a manner which is beneficial to infection and deleterious to the target cell, i.e., they perturb the normal function of the target cell. Examples of actions of virulence factors include, but are not limited to, modulation of actin polymerization, induction of apoptosis, modulation of the cell cycle, modulation of gene transcription. Not all substrates are necessarily virulence factors. By way of non-limiting example, a T3SS (and a functional T3SS) can comprise proteins referred to as translocators. These substrates are secreted by the T3SS as it nears a complete form and create a pore in the target cell membrane, allowing further substrates to be delivered into the cytoplasm of the target cell, i.e., translocators are substrates in that they travel through the needle to the target cell and are also structural proteins in that they form part of the structure through which other substrates are delivered into the target cell. In some embodiments, a single polypeptide can be both a translocator and a virulence factor (e.g. IpaB of Shigella). A functional T3SS system can be introduced into a non-pathogenic bacterial cell.

Homologs of any given polypeptide or nucleic acid sequence can be found using, e.g., BLAST programs (freely available on the world wide web at blast.ncbi.nlm.nih.gov/), e.g. by searching freely available databases of sequence for homologous sequences, or by querying those databases for annotations indicating a homolog (e.g. search strings that comprise a gene name or describe the activity of a gene). The homologous amino acid or DNA sequence can be at least 90%, at least 95%, at least 96%, at least 97%, at least 98%, at least 99%, or more, identical to a reference sequence. The degree of homology (percent identity) between a reference and a second sequence can be determined, for example, by comparing the two sequences using freely available computer programs commonly employed for this purpose on the world wide web.

Examples of T3SS secretion signals and chaperone-binding domains are known in the art, see, e.g. Schmitz et al. Nat Methods 2009 6:500-2; which described the signals and domains of Shigella effectors and which is incorporated by reference herein in its entirety. Additional examples are known in the art, e.g. Sory et al. PNAS 1995 92:11998-20002; which is is incorporated by reference herein in its entirety. It is contemplated that a T3SS signal may reduce the activity of the non-T3SS signal portion of the T3SS-compatible polypeptide once it is delivered to the target cell. Accordingly, in some embodiments, the T3SS-compatible polypeptide can comprise a cleavage site after the T3SS signal sequence. In some embodiments, the cleavage site is a site recognized by an endogenous component of the target cell, e.g. a calpain, sumo, and/or furin cleavage site. In some embodiments, instead of a cleavage site, the T3SS-compatible polypeptide can comprise a ubiquitin molecule after the T3SS signal sequence such that the ubiquitin molecule and the sequence N-terminal of it is removed from the remainder of the polypeptide by a eukaryotic target cell. In some embodiments, the first amino acid C-terminal of the ubiquitin molecule can be a methionine.

The T3SS-compatible polypeptide may be an antigen. An engineered microbial cell comprising a T3SS-compatible antigen polypeptide may be to a subject, e.g., orally.

In one aspect, described herein is a kit comprising an engineered microbial cell as described herein. In one aspect, described herein is a kit comprising an engineered microbial cell comprising a first nucleic acid sequence comprising genes encoding a functional type three secretion system (T3SS); and a second nucleic acid sequence encoding an T3SS-compatible polypeptide; wherein the engineered microbial cell is non-pathogenic with respect to a target cell. Citation or identification of any reference herein, in any section of this application, shall not be construed as an admission that such reference is available as prior art to the present application. The disclosures of each reference disclosed herein, whether U.S. or foreign patent literature, or non-patent literature, are hereby incorporated by reference in their entirety in this application, and shall be treated as if the entirety thereof forms a part of this application.

Such references are provided for their disclosure of technologies to enable practice of the present invention, to provide basis for claim language, to make clear applicant's possession of the invention with respect to the various aggregates, combinations, and subcombinations of the respective disclosures or portions thereof (within a particular reference or across multiple references). The citation of references is intended to be part of the disclosure of the invention, and not merely supplementary background information. The incorporation by reference does not extend to teachings which are inconsistent with the invention as expressly described herein, and is evidence of a proper interpretation by persons of ordinary skill in the art of the terms, phrase and concepts discussed herein, without being limiting as the sole interpretation available.

Genetically-engineered bacterial vectors represent a promising method of therapy for various diseases and as a biomolecule delivery system.

Tumor-targeted bacteria, especially those derived from wild type samples, are typically capable of producing a chronic infection without strong acute response. That is, these bacteria seem to have evolved to avoid triggering a debilitating immune response in the host while at the same time establishing long term colonization of tissues, in the case of tumor targeting bacteria, tissues which may include necrotic regions. According to some evolutionary theories, the attenuated host response to these bacteria may result from a survival benefit for the host in permitting the colonization. Indeed, there are at least anecdotal reports of successful eradication of tumors by bacterial therapy. This implies that bacteria derived from these strains can be pharmaceutically acceptable, for administration through various routes of administration.

Much research has been performed on bacterial therapies and bacterial delivery vectors. For example, tumor targeting bacteria offer tremendous potential advantages for the treatment of solid tumors, including the targeting from a distant inoculation site and the ability to express therapeutic agents directly within the tumor (Pawelek et al., 1997, Tumor-targeted Salmonella as a novel anticancer agent, Cancer Research 57: 4537-4544; Low et al., 1999, Lipid A mutant Salmonella with suppressed virulence and TNF-alpha induction retain tumor-targeting in vivo, Nature Biotechnol. 17: 37-41). However, the primary shortcoming of tumor-targeted bacteria investigated in the human clinical trials (Salmonella strain VNP20009 also known as YS1646, and its derivative TAPET-CD; Toso et al., 2002, Phase I study of the intravenous administration of attenuated Salmonella typhimurium to patients with metastatic melanoma, J. Clin, Oncol. 20: 142-152; Meir et al., 2001, Phase 1 trial of a live, attenuated Salmonella typhimurium (VNP20009) administered by direct Intra-tumoral (IT) injection, Proc Am Soc Clin Oncol 20: abstr 1043); Nemunaitis et al., 2003, Pilot trial of genetically modified, attenuated Salmonella expressing the E. coli cytosine deaminase gene in refractory cancer patients, Cancer Gene Therapy 10: 737-744) is that no significant antitumor activity has been observed, even in patients where the bacteria was documented to target the tumor. One method of increasing the ability of the bacteria to kill tumor cells is to engineer the bacteria to express conventional bacterial toxins (e.g., WO 2009/126189, WO 03/014380, WO/2005/018332, WO/2008/073148, US 2003/0059400 U.S. Pat. Nos. 7,452,531, 7,354,592, 6,962,696, 6,923,972, 6,863,894, 6,685,935, 6,475,482, 6,447,784, 6,190,657 and 6,080,849, 8,241,623, 8,524,220 8,771,669, 8,524,220).

Use of secreted proteins in live bacterial vectors has been demonstrated by several authors. Holland et al. (U.S. Pat. No. 5,143,830) have illustrated the use of fusions with the C-terminal portion of the hemolysin A (hlyA) gene, a member of the type I secretion system. When co-expressed in the presence of the hemolysin protein secretion channel (hlyBD) and a functional TolC, heterologous fusions are readily secreted from the bacteria. The type I secretion system that has been utilized most widely, and although it is currently considered the best system available, is thought to have limitations for delivery by attenuated bacteria (Hahn and Specht, 2003, FEMS Immunology and Medical Microbiology, 37: 87-98). Those limitations include the amount of protein secreted and the ability of the protein fused to it to interfere with secretion. Improvements of the type I secretion system have been demonstrated by Sugamata and Shiba (2005 Applied and Environmental Microbiology 71: 656-662), using a modified hlyB, and by Gupta and Lee (2008 Biotechnology and Bioengineering, 101: 967-974), by addition of rare codons to the hlyA gene. Fusion to the gene ClyA (Galen et al., 2004, Infection and Immunity, 72: 7096-7106 and Type III secretion proteins have also been used. Surface display has been used to export proteins outside of the bacteria. For example, fusion of the Lpp protein amino acids 1-9 with the transmembrane region B3-B7 of OmpA has been used for surface display (Samuelson et al., 2002, Display of proteins on bacteria, J. Biotechnology 96: 129-154). The autotransporter surface display has been described by Berthet et al., WO/2002/070645.

Other heterologous protein secretion systems utilizing the autotransporter family can be modulated to result in either surface display or complete release into the medium (see Henderson et al., 2004, Type V secretion pathway: the autotransporter story, Microbiology and Molecular Biology Reviews 68: 692-744; Jose, 2006 Applied Microbiol. Biotechnol. 69: 607-614; Jose J, Zangen D (2005) Autodisplay of the protease inhibitor aprotinin in Escherichia coli. Biochem Biophys Res Commun 333:1218-1226 and Rutherford and Mourez 2006 Microbial Cell Factories 5: 22). For example, Veiga et al. (2003 Journal of Bacteriology 185: 5585-5590 and Klauser et al., 1990 EMBO Journal 9: 1991-1999), demonstrated hybrid proteins containing the b-autotransporter domain of the immunoglobulin A (IgA) protease of Nisseria gonorrhea. Fusions to flagellar proteins have been demonstrated. The peptide, usually of 15 to 36 amino acids in length, is inserted into the central, hypervariable region of the FliC gene such as that from Salmonella muenchen (Verma et al. 1995 Vaccine 13: 235-24; Wu et al., 1989 Proc. Natl. Acad. Sci. USA 86: 4726-4730; Cuadro et al., 2004 Infect. Immun. 72: 2810-2816; Newton et al., 1995, Res. Microbiol. 146: 203-216, each of which is expressly incorporated by reference in its entirety). Multihybrid FliC insertions of up to 302 amino acids have also been prepared (Tanskanen et al. 2000, Appl. Env. Microbiol. 66: 4152-4156). Trimerization of antigens and functional proteins can be achieved using the T4 fibritin foldon trimerization sequence (Wei et al. 2008 J. Virology 82: 6200-6208) and VASP tetramerization domains (Kühnel et al., 2004 PNAS 101: 17027-17032). The multimerization domains are used to create, bi-specific, tri-specific, and quatra-specific targeting agents, whereby each individual agent is expressed with a multimerization tag, each of which may have the same or separate targeting peptide, such that following expression, surface display, secretion and/or release, they form multimers with multiple targeting domains. Other secretion systems include C-terminal fusions to the protein YebF (Zhang et al., 2006, Extracellular accumulation of recombinant proteins fused to the carrier protein YebF in Escherichia coli, Nat Biotechnol 24: 100-104), which is commercially available as a kit (pAES40; AthenaES, Baltimore, Md.). Fusions to OmsY and other proteins are also capable of secreting proteins into the medium (Zian et al., 2008, Proteome-Based Identification of Fusion Partner for High-Level Extracellular Production of Recombinant Proteins in Escherichia coli, Biotechnol Bioegineer 101: 587-601). Other secretions systems usable according to the present invention include that of Kotzsch et al. 2011 (A secretory system for bacterial production of high-profile protein targets, Protein Science 20: 597-609) using OmpA, OmpF and OsmY, or those described by Yoon et al., 2010 (Secretory production of recombinant proteins in Escherichia coli, Recent Patents on Biotechnology 4: 23-29. See, US2006-7094579B2, WO2009021548A1, EP1402036B1, US2006-7070989B2, US2008/0193974A1, US2006-7052867B2, US2003-6605697B1, U.S. Pat. No. 5,470,719A, US2007/0287171A1, US2009/0011995A1, US2008/0076157A1, US2006-7112434B2, US2005-6919198B1, US2002-6455279B1, US2007-7291325B2, US2008-7410788B2, US2000-6083715A, EP1270730A1, US2004-6673569B1, US2001-6309861B1, U.S. Pat. No. 5,989,868A, US2006-7056732B2, US2005-6852512B2, US2005-6861403B2, EP1407052B1, WO2008089132A2, U.S. Pat. No. 5,824,502A, EP1068339B1, US2008/0166757A1, US2001-6329172B1, US2003-6596509B1, US2003-6642027B2, WO2006017929A1, US2003-6596510B1, US2008/0280346A1, US2007-7202059B2, US2008/0280346A1, US2007-7202059B2, US2009-7491528B2, US2008/0206814A1, US2008/0166764A1, US2008/0182295A1, US2008/0254511A1, US2008/0206818A1, US2006-7105327B1, US2004/0005695A1, U.S. Pat. No. 5,508,192, EP866132A2, U.S. Pat. Nos. 6,921,659B2, 6,828,121B2, US2008/0064062A1, EP786009B1, US2006/0270043A1, and U.S. Pat. No. 7,202,059.

Compositions described in accordance with various embodiments herein include, without limitation, Salmonella enterica serovar Typhimurium (“S. typhimurium”), Salmonella montevideo, Salmonella enterica serovar Typhi (“S. typhi”), Salmonella enterica serovar Paratyphi A, Paratyphi B (“S. paratyphi 13”), Salmonella enterica serovar Paratyphi C (“S. paratyphi C”), Salmonella enterica serovar Hadar (“S. hadar”), Salmonella enterica serovar Enteriditis (“S. enteriditis”), Salmonella enterica serovar Kentucky (“S. kentucky”), Salmonella enterica serovar Infantis (“S. infantis”), Salmonella enterica serovar Pullorum (“S. pullorum”), Salmonella enterica serovar Gallinarum (“S. gallinarum”), Salmonella enterica serovar Muenchen (“S. muenchen”), Salmonella enterica serovar Anaturn (“S. anatum”), Salmonella enterica serovar Dublin (“S. dublin”), Salmonella enterica serovar Derby (“S. derby”), Salmonella enterica serovar Choleraesuis var. kunzendorf (“S. cholerae kunzendorf”), and Salmonella enterica serovar minnesota (S. minnesota).

By way of example, live bacteria in accordance with aspects of the invention include known strains of S. enterica serovar Typhimurium (S. typhimurium) and S. enterica serovar Typhi (S. typhi) which are further modified as provided by various embodiments of the invention. Such Strains include Ty21a, CMV906, CMV908, CMV906-htr, CMV908-htr, Ty800, aroA-/serC-, holavax, M01ZH09, VNP20009. These strains contain defined mutations within specific serotypes of bacteria. The technology also includes the use of these same (or different) mutational combinations contained within alternate serotypes or strains in order to avoid immune reactions which may occur in subsequent administrations. For example, S. typhimurium, S. montevideo, and S. typhi which have non-overlapping O-antigen presentation (e.g., S. typhimurium is O—1, 4, 5, 12 and S. typhi is Vi, S. montevideo is O—6, 7) may be used. Thus, for example, S. typhimurium is a suitable serotype for a first administration and another serotype such as S. typhi or S. montevideo are used for a second administration and third administration. Likewise, the flagellar antigens are also selected for non-overlapping antigenicity between different administrations. The flagellar antigen may be H1 or H2 or no flagellar antigen, which, when combined with the three different O-antigen serotypes, provides three completely different antigenic profiles.

Winter, K, Xing, L. and Ward, B. G., McGill University, “Attenuated Salmonella typhimurium as a vector for a novel Clostridium difficile vaccine”, Abstract II 084, CSM 2017 Poster Session, 67th Annual Conference of the Canadian Society of Microbiologists, University of Waterloo, Waterloo, Ontario, June 20th-Jun. 23, 2017, earlier work of the inventors suggest that attenuated Salmonella enterica species are attractive as vaccine vectors due to their potential to induce both local (mucosal) and systemic immune responses. To facilitate stimulation of immune responses, type III secretion systems (T3SS) of Salmonella can be employed to deliver heterologous antigens to antigen-presenting cells. The genome of S. enterica contains two loci termed Salmonella pathogenicity island 1 and 2 (SPI-I and SPI-II) that encode distinct T3SS that translocate effector proteins at the different stages of Salmonella infection. While these secretion systems have been exploited previously to deliver foreign antigens in Salmonella-based vaccine development efforts, the distinct spatial and temporal functions of the SPI-I and SPI-II systems on immune responses, particularly in terms of mucosal immunity, have yet to be systemically investigated. Proposed antigenic targets are the C-terminal receptor binding domains (RBDs) of Clostridium difficile toxins A and B (TcdA, TcdB). Anti-RBD antibodies have been shown to protect against C. difficile infection in both animal models and humans. A panel of 13 vaccine candidates has been developed based on a well-characterized, attenuated S. typhimurium strain (YS1646) that express the RBDs of either TcdA or TcdB using different SPI-I and SPI-II promoters and secretory signals. Western Blot and immunofluorescence results show that expression of these antigens is variable in vitro, both when the bacteria is grown in LB broth and upon invasion of a mouse macrophage cell line (RAW264.7).

Preliminary data in a mouse vaccination model (3 doses of 10⁹ bacteria by gavage either every other day or every 2 weeks) suggest that several of these vaccine candidate that exploit different SPI-I and SPI-II T3SS promoters and secretory signals elicit systemic immune responses at least (IgG by ELISA). The vaccine schedule was not optimized to find the construct that elicit both systemic and mucosal immunity (serum IgG, stool fluid IgA, cellular responses). Thus, while it was shown that YS1646 could be used to produce vaccine candidates with TcdA and TcdB antigens secreted by the SPI-I or SPI-II T3SS system, and that these could raise IgG immune responses in mice, the existence of IgA response or protective immunity was not demonstrated, and required seven doses of bacteria. See also, Wang, Yuanguo; Wang, Shaohui; Bouillaut, Laurent; Li, Chunhui; Duan, Zhibian; Zhang, Keshan; Tzipori, Saul; Sonenshein, Abraham; Sun, Xingmin. (2018). Oral immunization with non-toxic C. difficile strains expressing chimeric fragments of TcdA and TcdB elicits protective immunity against C. difficile infection in both mice and hamsters. Infection and Immunity. 10.1128/IAI.00489-18.

See also, U.S. Pat. No. 6,548,287, and EP0973911. See also, US 20140256922; 20120108640; 20110318308; 20090215754; 20090169517; 20070298012; 20070110752; 20070004666; 20060115483; 20060104955; 20060089350; 20060025387; 20050267103; 20050249706; 20050112642; 20050009750; 20040229338; 20040219169; 20040058849; 20030143676; 20030113293; 20030031628; 20030022835; 20020151063; 20140220661; 20140212396; 20140186401; 20140178341; 20140155343; 20140093885; 20130330824; 20130295054; 20130209405; 20130130292; 20120164687; 20120142080; 20120128594; 20120093773; 20120020883; 20110275585; 20110111496; 20110111481; 20100239546; 20100189691; 20100136048; 20100135973; 20100135961; 20100092438; 20090300779; 20090180955; 20090175829; 20090123426; 20090053186; 20080311081; 20080124355; 20080038296; 20070110721; 20070104689; 20060083716; 20050026866; 20050008618; 20040202663; 20050255088; 20030109026; 20020026655; 20110223241; 20070009489; 20050036987; 20030170276; 20140148582; 20130345114; 20130287810; 20130164380; 20130164307; 20130078275; 20120225454; 20120177682; 20120148601; 20120144509; 20120083587; 20120021517; 20110274719; 20110268661; 20110165680; 20110091493; 20110027349; 20100172976; 20090317404; 20090220540; 20090123382; 20090117049; 20090117048; 20090117047; 20090068226; 20080249013; 20080206284; 20070202591; 20070191262; 20070134264; 20060127408; 20060057152; 20050118193; 20050069491; 20050064526; 20040234455; 20040202648; 20040054142; 20030170211; 20030059400; 20030036644; 20030009015; 20030008839; 20020176848; 20020102242; 20140205538; 20140112951; 20140086950; 20120244621; 20120189572; 20110104196; 20100233195; 20090208534; 20090136542; 20090028890; 20080260769; 20080187520; 20070031382; 20060140975; 20050214318; 20050214317; 20050112140; 20050112139; 20040266003; 20040115174; 20040009936; 20030153527; 20030125278; 20030045492; U.S. Pat. Nos. 8,828,681; 8,822,194; 8,784,836; 8,771,669; 8,734,779; 8,722,668; 8,715,641; 8,703,153; 8,685,939; 8,663,634; 8,647,642; 8,642,257; 8,623,350; 8,604,178; 8,591,862; 8,586,022; 8,568,707; 8,551,471; 8,524,220; 8,440,207; 8,357,486; 8,343,509; 8,323,959; 8,282,919; 8,241,623; 8,221,769; 8,198,430; 8,137,904; 8,066,987; 8,021,662; 8,008,283; 7,998,461; 7,955,600; 7,939,319; 7,915,218; 7,887,816; 7,842,290; 7,820,184; 7,803,531; 7,790,177; 7,786,288; 7,763,420; 7,754,221; 7,740,835; 7,736,898; 7,718,180; 7,700,104; 7,691,383; 7,687,474; 7,662,398; 7,611,883; 7,611,712; 7,588,771; 7,588,767; 7,514,089; 7,470,667; 7,452,531; 7,404,963; 7,393,525; 7,354,592; 7,344,710; 7,247,296; 7,195,757; 7,125,718; 7,084,105; 7,083,791; 7,015,027; 6,962,696; 6,923,972; 6,916,918; 6,863,894; 6,770,632; 6,685,935; 6,682,729; 6,506,550; 6,500,419; 6,475,482; 6,447,784; 6,207,648; 6,190,657; 6,150,170; 6,080,849; 6,030,624; and 5,877,159.

Novel strains are also encompassed that are, for example, attenuated in virulence by mutations in a variety of metabolic and structural genes. The invention therefore may provide a live composition for treating cancer comprising a live attenuated bacterium that is a serovar of Salmonella enterica comprising an attenuating mutation in a genetic locus of the chromosome of said bacterium that attenuates virulence of said bacterium and wherein said attenuating mutation is the Suwwan deletion (Murray et al., 2004. Hot spot for a large deletion in the 18-19 Cs region confers a multiple phenotype in Salmonella enterica serovar Typhimurium strain ATCC 14028. Journal of Bacteriology 186: 8516-8523 (2004)) or combinations with other known attenuating mutations. Other attenuating mutation useful in the Salmonella bacterial strains described herein may be in a genetic locus selected from the group consisting of phoP, phoQ, edt, cya, crp, poxA, rpoS, htrA, nuoG, pmi, pabA, pts, damA, pur, purA, purB, purI, purF, zwf, aroA, aroB, aroC, aroD, serC, gua, cadA, rfc, rjb, rfa, ompR, msbB, leucine and arginine, and combinations thereof. Strains of Salmonella deleted in stn are particularly preferred.

Attenuated gram-positive bacteria are also available as delivery vectors. For example, Staphylococcus epidermidis, group B Streptococcus including S. agalaciae, and Listeria species including L. monocytogenes may be employed. It is known to those skilled in the art that variations in molecular biology techniques such as use of gram-positive origins of replication, gram-positive signal sequences and gram-positive promoters and filamentous phage (e.g., phage B5; Chopin et al., 2002 J. Bacteriol. 184: 2030-2033, described further below) may be employed and substituted as needed. Other bacterial strains may also be encompassed, including non-pathogenic bacteria of the gut skin (such as Staphylococcus epidermidis, Proprionibacteria) and other body locations known as the human microbiome (Grice et al., Topographical and temporal diversity of the human skin microbiome, Science 324: 1190-1192; A framework for human microbiome research; The Human Microbiome Project Consortium, 14 Jun. 2012 Nature 486, 215-221; Spor et al., 2011, Unravelling the effects of the environment and host genotype on the gut microbiome, Nature Reviews Microbiology 9: 279-290) such as E. coli strains, Bacteroides, Bifidobacterium and Bacillus, attenuated pathogenic strains of E. coli including enteropathogenic and uropathogenic isolates, Enterococcus sp. and Serratia sp. as well as attenuated Neisseria sp., Shigella sp., Staphylococcus sp., Staphylococcus carnosis, Yersinia sp., Streptococcus sp. and Listeria sp. including L. monocytogenes. Bacteria of low pathogenic potential to humans and other mammals or birds or wild animals, pets and livestock, such as insect pathogenic Xenorhabdus sp., Photorhabdus sp. and human wound Photorhabdus (Xenorhabdus) are also encompassed. Probiotic strains of bacteria are also encompassed, including Lactobacillus sp. (e.g., Lactobacillus acidophilus, Lactobacillus salivarius) Lactococcus sp., (e.g., Lactococcus lactis, Lactococcus casei) Leuconostoc sp., Pediococcus sp., Streptococcus sp. (e.g., S. salivariu, S. thermophilus), Bacillus sp., Bifidobacterium sp., Bacteroides sp., and Escherichia coli such as the 1917 Nissel strain.

It is known to those skilled in the art that minor variations in molecular biology techniques such as use of gram-positive origins of replication, gram-positive signal sequences gram-positive promoters (e.g., Lactococcus expression, Mohamadzadeh et al., PNAS Mar. 17, 2009 vol. 106 no. 11 4331-4336) may be used and substituted as needed. The bacteria may be further modified to be internalized into the host cell (Guimaraes et al., 2006, Use of Native Lactococci as Vehicles for Delivery of DNA into Mammalian Epithelial Cells, Appl Environ Microbiol. 2006 November; 72(11): 7091-7097; Innocentin et al., 2009, Lactococcus lactis Expressing either Staphylococcus aureus Fibronectin-Binding Protein A or Listeria monocytogenes Internalin A Can Efficiently Internalize and Deliver DNA in Human Epithelial Cells Appl Environ Microbiol. 2009 July; 75(14): 4870-4878).

Each of the following is also expressly incorporated herein by reference in its entirety:

U.S. Pat. Nos. 4,190,495; 4,888,170; 4,968,619; 5,066,596; 5,098,998; 5,294,441; 5,330,753; 5,387,744; 5,424,065; 5,468,485; 5,527,678; 5,627,067; 5,628,996; 5,643,771; 5,654,184; 5,656,488; 5,662,905; 5,672,345; 5,679,880; 5,686,079; 5,695,983; 5,717,071; 5,731,196; 5,736,367; 5,747,028; 5,770,214; 5,773,007; 5,811,105; 5,824,538; 5,830,702; 5,837,509; 5,837,541; 5,840,483; 5,843,426; 5,855,879; 5,855,880; 5,869,066; 5,874,088; 5,877,159; 5,888,799; 6,024,961; 6,051,416; 6,077,678; 6,080,849; 6,100,388; 6,129,917; 6,130,082; 6,150,170; 6,153,203; 6,177,083; 6,190,657; 6,207,167; 6,245,338; 6,254,875; 6,284,477; 6,294,655; 6,337,072; 6,339,141; 6,365,163; 6,365,723; 6,365,726; 6,372,892; 6,383,496; 6,410,012; 6,413,523; 6,426,191; 6,444,445; 6,447,784; 6,471,964; 6,475,482; 6,495,661; 6,500,419; 6,506,550; 6,511,666; 6,531,313; 6,537,558; 6,541,623; 6,566,121; 6,593,147; 6,599,509; 6,610,300; 6,610,529; 6,653,128; 6,682,729; 6,685,935; 6,719,980; 6,737,521; 6,749,831; 6,752,994; 6,780,405; 6,825,028; 6,855,814; 6,863,894; 6,872,547; 6,887,483; 6,905,691; 6,913,753; 6,916,478; 6,923,958; 6,923,972; 6,962,696; 6,992,237; 6,994,860; 7,005,129; 7,018,835; 7,026,155; 7,045,336; 7,056,700; 7,063,850; 7,083,794; 7,094,410; 7,115,269; 7,144,580; 7,144,982; 7,183,105; 7,195,757; 7,226,588; 7,235,234; 7,264,812; 7,279,464; 7,341,725; 7,341,841; 7,341,860; 7,354,592; 7,393,525; 7,407,790; 7,425,438; 7,452,531; 7,459,161; 7,473,247; 7,510,717; 7,514,089; 7,514,415; 7,531,723; 7,541,043; 7,569,219; 7,569,552; 7,569,682; 7,588,767; 7,588,771; 7,601,804; 7,622,107; 7,625,572; 7,657,380; 7,662,398; 7,666,656; 7,691,393; 7,695,725; 7,700,091; 7,700,104; 7,718,179; 7,732,187; 7,754,221; 7,758,876; 7,763,420; 7,772,386; 7,776,527; 7,794,734; 7,803,531; 7,803,990; 7,807,184; 7,807,456; 7,820,184; 7,829,104; 7,833,775; 7,842,289; 7,842,290; 7,850,958; 7,850,970; 7,871,604; 7,871,815; 7,871,816; 7,887,816; 7,919,081; 7,927,606; 7,930,107; 7,951,386; 7,951,786; 7,955,600; 7,960,518; 7,972,604; 7,985,573; 7,993,651; 8,012,466; 8,021,662; 8,021,848; 8,034,359; 8,043,857; 8,048,428; 8,049,000; 8,053,181; 8,053,421; 8,066,987; 8,071,084; 8,071,319; 8,076,099; 8,101,396; 8,114,409; 8,114,414; 8,124,068; 8,124,408; 8,133,493; 8,137,904; 8,147,820; 8,168,421; 8,173,773; 8,187,610; 8,202,516; 8,207,228; 8,211,431; 8,221,769; 8,227,584; 8,241,623; 8,241,631; 8,241,637; 8,257,713; 8,273,361; 8,287,883; 8,288,359; 8,318,661; 8,323,668; 8,323,959; 8,329,685; 8,337,832; 8,337,861; 8,343,509; 8,343,512; 8,349,586; 8,357,486; 8,357,533; 8,361,707; 8,367,055; 8,399,618; 8,440,207; 8,445,254; 8,445,426; 8,445,662; 8,460,666; 8,465,755; 8,470,551; 8,481,055; 8,501,198; 8,524,220; 8,551,497; 8,557,789; 8,568,707; 8,580,280; 8,586,022; 8,591,862; 8,609,114; 8,623,350; 8,628,776; 8,632,783; 8,633,305; 8,642,257; 8,642,656; 8,647,642; 8,658,350; 8,663,940; 8,669,355; 8,673,311; 8,679,505; 8,703,153; 8,715,929; 8,716,254; 8,716,343; 8,722,064; 8,748,150; 8,758,766; 8,771,669; 8,772,013; 8,778,683; 8,784,829; 8,784,836; 8,790,909; 8,840,908; 8,853,382; 8,859,256; 8,877,212; 8,883,147; 8,889,121; 8,889,150; 8,895,062; 8,916,372; 8,926,993; 8,937,074; 8,951,531; 8,956,618; 8,956,621; 8,956,859; 8,961,989; 8,980,279; 8,992,943; 9,005,665; 9,011,870; 9,012,213; 9,017,986; 9,023,635; 9,040,059; 9,040,233; 9,045,528; 9,045,742; 9,050,285; 9,050,319; 9,051,574; 9,056,909; 9,062,297; 9,068,187; 9,107,864; 9,140,698; 9,161,974; 9,163,219; 9,169,302; 9,173,930; 9,173,935; 9,173,936; 9,180,183; 9,181,546; 9,198,960; 9,200,251; 9,200,289; 9,205,142; 9,220,764; 9,248,177; 9,255,149; 9,255,283; 9,265,804; 9,267,108; 9,289,481; 9,297,015; 9,303,264; 9,309,493; 9,315,817; 9,320,787; 9,320,788; 9,333,251; 9,339,533; 9,358,283; 9,364,528; 9,365,625; 9,376,686; 9,408,880; 9,415,077; 9,415,098; 9,421,252; 9,428,572; 9,441,204; 9,453,227; 9,457,074; 9,457,077; 9,463,238; 9,474,831; 9,480,740; 9,481,884; 9,481,888; 9,486,513; 9,487,577; 9,492,534; 9,499,606; 9,504,750; 9,506,922; 9,526,778; 9,529,005; 9,539,313; 9,540,407; 9,546,199; 9,549,956; 9,556,442; 9,561,270; 9,562,080; 9,562,837; 9,566,321; 9,566,322; 9,567,375; 9,580,478; 9,580,718; 9,592,283; 9,593,339; 9,597,379; 9,598,697; 9,603,799; 9,610,342; 9,616,114; 9,622,486; 9,636,386; 9,642,881; 9,642,904; 9,649,345; 9,651,559; 9,655,815; 9,657,085; 9,657,327; 9,662,385; 9,663,758; 9,670,270; 9,695,229; 9,714,426; 9,717,782; 9,730,996; 9,737,592; 9,737,601; 9,739,773; 9,750,802; 9,758,572; 9,764,021; 9,775,896; 9,795,641; 9,796,762; 9,801,930; 9,808,517; 9,814,772; 9,827,305; 9,844,592; 9,845,342; 9,855,336; 9,856,311; 9,867,785; 9,872,898; 9,878,023; 9,878,024; 9,878,043; 9,884,108; 9,885,051; 9,889,165; 9,901,082; 9,907,755; 9,907,845; 9,913,893; 9,925,257; 9,950,063; 9,986,724; 9,987,355; 9,994,809; 9,999,660; 20010014673; 20020025325; 20020028215; 20020044938; 20020068068; 20020076417; 20020077272; 20020081317; 20020086032; 20020086332; 20020090376; 20020132789; 20020146430; 20020151462; 20020156009; 20020176848; 20030017162; 20030023075; 20030045492; 20030065039; 20030068328; 20030100100; 20030108562; 20030108957; 20030124516; 20030125278; 20030130827; 20030152589; 20030153527; 20030157637; 20030166099; 20030166279; 20030170211; 20030170613; 20030176377; 20030180260; 20030180304; 20030180320; 20030185802; 20030186908; 20030190601; 20030190683; 20030190749; 20030194714; 20030194755; 20030194798; 20030198995; 20030198996; 20030199005; 20030199088; 20030199089; 20030202937; 20030203411; 20030203481; 20030207833; 20030211086; 20030211103; 20030211461; 20030211476; 20030211599; 20030219408; 20030219888; 20030224369; 20030224444; 20030232335; 20030235577; 20040005700; 20040009540; 20040009936; 20040013658; 20040013689; 20040023310; 20040033539; 20040052817; 20040053209; 20040077067; 20040121307; 20040121474; 20040126871; 20040131641; 20040132678; 20040137003; 20040156865; 20040192631; 20040202663; 20040213804; 20040228877; 20040229338; 20040237147; 20040258703; 20040258707; 20040265337; 20050008618; 20050010032; 20050026866; 20050042755; 20050048076; 20050075298; 20050106151; 20050106176; 20050129711; 20050163791; 20050175630; 20050180985; 20050222057; 20050229274; 20050232937; 20050233408; 20050249706; 20050249752; 20050255125; 20050271643; 20050271683; 20050281841; 20050287123; 20060018877; 20060019239; 20060074039; 20060078572; 20060083716; 20060115494; 20060121045; 20060121054; 20060140971; 20060140975; 20060147418; 20060147461; 20060153875; 20060171960; 20060182754; 20060189792; 20060193874; 20060233835; 20060240494; 20060246083; 20060257415; 20060269570; 20070031382; 20070031458; 20070037225; 20070048331; 20070059323; 20070104733; 20070104736; 20070110717; 20070116725; 20070122881; 20070128216; 20070134214; 20070134272; 20070141082; 20070154495; 20070169226; 20070189982; 20070258901; 20070281328; 20070286874; 20070298012; 20080008718; 20080020441; 20080038296; 20080063655; 20080095794; 20080107653; 20080112974; 20080124355; 20080131466; 20080138359; 20080181892; 20080188436; 20080193373; 20080213308; 20080241179; 20080241858; 20080254058; 20080261869; 20080286852; 20080311081; 20080317742; 20090017000; 20090017048; 20090028892; 20090053186; 20090074816; 20090081250; 20090081257; 20090104204; 20090117151; 20090117152; 20090123426; 20090142310; 20090148473; 20090169517; 20090169562; 20090180987; 20090181078; 20090196887; 20090214476; 20090227013; 20090253778; 20090263414; 20090263418; 20090285844; 20090297552; 20090297561; 20090304750; 20090305398; 20090324503; 20090324576; 20090324638; 20090324641; 20100047286; 20100055082; 20100055127; 20100068214; 20100092438; 20100092518; 20100099600; 20100129406; 20100135961; 20100136048; 20100136055; 20100136058; 20100137192; 20100166786; 20100166800; 20100172938; 20100172976; 20100189691; 20100196524; 20100209446; 20100226891; 20100226931; 20100226941; 20100233212; 20100233213; 20100239546; 20100272748; 20100272759; 20100285592; 20100291148; 20100297184; 20100297740; 20100303862; 20100310602; 20100322957; 20110008389; 20110014274; 20110020401; 20110021416; 20110052628; 20110059126; 20110064723; 20110064766; 20110070290; 20110086059; 20110104186; 20110110979; 20110111481; 20110111496; 20110123565; 20110183342; 20110195093; 20110200631; 20110201092; 20110201676; 20110206694; 20110209228; 20110212090; 20110213129; 20110217323; 20110243992; 20110256214; 20110268739; 20110275585; 20110281330; 20110287046; 20110293662; 20110312020; 20120003298; 20120009247; 20120014881; 20120027811; 20120036589; 20120039931; 20120039994; 20120058142; 20120071545; 20120077206; 20120093773; 20120093850; 20120093865; 20120100177; 20120107340; 20120115223; 20120121647; 20120135039; 20120135503; 20120141493; 20120142079; 20120142080; 20120144509; 20120164687; 20120189657; 20120189661; 20120208866; 20120225454; 20120237491; 20120237537; 20120237544; 20120258128; 20120258129; 20120258135; 20120276167; 20120282181; 20120282291; 20120288523; 20120294948; 20120301422; 20120315278; 20130004547; 20130018089; 20130040370; 20130058997; 20130064845; 20130078275; 20130078278; 20130084307; 20130095131; 20130096103; 20130101523; 20130110249; 20130121968; 20130149321; 20130156809; 20130177589; 20130177593; 20130217063; 20130236948; 20130251719; 20130266635; 20130267481; 20130273144; 20130302380; 20130315950; 20130330824; 20130336990; 20130337012; 20130337545; 20140004178; 20140004193; 20140010844; 20140017279; 20140017285; 20140037691; 20140056940; 20140065187; 20140093477; 20140093954; 20140099320; 20140112951; 20140134662; 20140155343; 20140178425; 20140186398; 20140186401; 20140187612; 20140193459; 20140206064; 20140212396; 20140220661; 20140234310; 20140234379; 20140271563; 20140271719; 20140294883; 20140322249; 20140322265; 20140322267; 20140322268; 20140335125; 20140341921; 20140341942; 20140341970; 20140341974; 20140356415; 20140369986; 20140370036; 20140370057; 20140371428; 20150017138; 20150017191; 20150017204; 20150030573; 20150037370; 20150050311; 20150056246; 20150071994; 20150093824; 20150125485; 20150125921; 20150132335; 20150140028; 20150140034; 20150140037; 20150165011; 20150174178; 20150182611; 20150184167; 20150190500; 20150196659; 20150202276; 20150204845; 20150218254; 20150219645; 20150225692; 20150238589; 20150258190; 20150265696; 20150273045; 20150316567; 20150321037; 20150335736; 20150343050; 20150376242; 20160000896; 20160022592; 20160030494; 20160045591; 20160054299; 20160058860; 20160074505; 20160090395; 20160101168; 20160103127; 20160108096; 20160136285; 20160136294; 20160158334; 20160158335; 20160169921; 20160175415; 20160175428; 20160193256; 20160193257; 20160199422; 20160199474; 20160206727; 20160208261; 20160213770; 20160220652; 20160222393; 20160228523; 20160228530; 20160243204; 20160250311; 20160263209; 20160289287; 20160317637; 20160324783; 20160324939; 20160346381; 20160354462; 20160366862; 20160367650; 20160369282; 20170007683; 20170014513; 20170015735; 20170021011; 20170028048; 20170042987; 20170042996; 20170051260; 20170072042; 20170080078; 20170081642; 20170081671; 20170095548; 20170106028; 20170106074; 20170114319; 20170129942; 20170136102; 20170136111; 20170143815; 20170145061; 20170145065; 20170151321; 20170157232; 20170157239; 20170174746; 20170182155; 20170191058; 20170209502; 20170216378; 20170240615; 20170246281; 20170258885; 20170290889; 20170290901; 20170304434; 20170318817; 20170327830; 20170340720; 20170350890; 20170360540; 20170368156; 20170368166; 20180008701; 20180021424; 20180028642; 20180028649; 20180043021; 20180044406; 20180049413; 20180050099; 20180066041; 20180066225; 20180071377; 20180087060; 20180099999; 20180104328; 20180140665; 20180147278; 20180164221; 20180168488; 20180168489; 20180168490; 20180169222; 20180169226; 20180185469; 20180193003; 20180193441; 20180206726; 20180206769; 20180221286; 20180221470; 20180236063; 20180243347; and 20180243348.

Amdekar, Sarika, Deepak Dwivedi, Purabi Roy, Sapna Kushwah, and Vinod Singh. “Probiotics: multifarious oral vaccine against infectious traumas.” FEMS Immunology & Medical Microbiology 58, no. 3 (2010): 299-306.

Ananthakrishnan, A. N. Clostridium difficile infection: epidemiology, risk factors and management. Nature Reviews Gastroenterology and Hepatology 8, 17-26, doi:10.1038/nrgastro.2010.190 (2010).

Arnold, Heinz, Dirk Bumann, Melanie Felies, Britta Gewecke, Meike Sörensen, J. Engelbert Gessner, Joachim Freihorst, Bernd Ulrich Von Specht, and Ulrich Baumann. “Enhanced immunogenicity in the murine airway mucosa with an attenuated Salmonella live vaccine expressing OprF-OprI from Pseudomonas aeruginosa.” Infection and immunity 72, no. 11 (2004): 6546-6553.

Aslam, Saima, Richard J. Hamill, and Daniel M. Musher. “Treatment of Clostridium difficile-associated disease: old therapies and new strategies.” The Lancet infectious diseases 5, no. 9 (2005): 549-557.

Avezov, E. et al. Lifetime imaging of a fluorescent protein sensor reveals surprising stability of ER thiol redox. The Journal of cell biology 201, 337-349, doi:10.1083/jcb.201211155 (2013).

Baliban, S. M. et al. An optimized, synthetic DNA vaccine encoding the toxin A and toxin B receptor binding domains of Clostridium difficile induces protective antibody responses in vivo. Infect. Immun. 82, 4080-4091, doi:10.1128/IAI.01950-14 (2014).

Baud, David, Françoise Ponci, Martine Bobst, Pierre De Grandi, and Denise Nardelli-Haefliger. “Improved efficiency of a Salmonella-based vaccine against human papillomavirus type 16 virus-like particles achieved by using a codon-optimized version of L1.” Journal of virology 78, no. 23 (2004): 12901-12909.

Bermúdez-Humarán, Luis G. “Lactococcus lactis as a live vector for mucosal delivery of therapeutic proteins.” Human vaccines 5, no. 4 (2009): 264-267.

Best, E. L., Freeman, J. & Wilcox, M. H. Models for the study of Clostridium difficile infection. Gut microbes 3, 145-167, doi:10.4161/gmic.19526 (2012).

Bézay, N. et al. Safety, immunogenicity and dose response of VLA84, a new vaccine candidate against Clostridium difficile, in healthy volunteers. Vaccine, doi:10.1016/j.vaccine.2016.03.098 (2016).

Blisnick, Thierry, Patrick Ave, Michel Huerre, Elisabeth Carniel, and Christian E. Demeure. “Oral vaccination against bubonic plague using a live avirulent Yersinia pseudotuberculosis strain.” Infection and immunity 76, no. 8 (2008): 3808-3816.

Bolhassani, Azam, and Farnaz Zahedifard. “Therapeutic live vaccines as a potential anticancer strategy.” International journal of cancer 131, no. 8 (2012): 1733-1743.

Branger, Christine G., Roy Curtiss III, Robert D. Perry, and Jacqueline D. Fetherston. “Oral vaccination with different antigens from Yersinia pestis KIM delivered by live attenuated Salmonella typhimurium elicits a protective immune response against plague.” In The Genus Yersinia, pp. 387-399. Springer, New York, N.Y., 2007.

Bruhn, Kevin W., Noah Craft, and Jeff F. Miller. “Listeria as a vaccine vector.” Microbes and infection 9, no. 10 (2007): 1226-1235.

Bruxelle, Jean-François, Séverine Péchiné, and Anne Collignon. “Immunization Strategies Against Clostridium difficile.” In Updates on Clostridium difficile in Europe, pp. 197-225. Springer, Cham, 2018. doi:10.1007/978-3-319-72799-8_12.

Bumann, Dirk. “In vivo visualization of bacterial colonization, antigen expression, and specific T-cell induction following oral administration of live recombinant Salmonella enterica serovar Typhimurium.” Infection and immunity 69, no. 7 (2001): 4618-4626.

Cardenas, Lucia, and J. D. Clements. “Oral immunization using live attenuated Salmonella spp. as carriers of foreign antigens.” Clinical microbiology reviews 5, no. 3 (1992): 328-342.

Carter, G. P. et al. Defining the Roles of TcdA and TcdB in Localized Gastrointestinal Disease, Systemic Organ Damage, and the Host Response during Clostridium difficile Infections. mBio 6, doi:10.1128/mBio.00551-15 (2015).

Carter, G. P., Rood, J. I. & Lyras, D. The role of toxin A and toxin B in Clostridium difficile-associated disease: Past and present perspectives. Gut microbes 1, 58-64, doi:10.4161/gmic.1.1.10768 (2010).

Chabalgoity, José A., Gordon Dougan, Pietro Mastroeni, and Richard J. Aspinall. “Live bacteria as the basis for immunotherapies against cancer.” Expert review of vaccines1, no. 4 (2002): 495-505.

Cheminay, Cédric, Annette Möhlenbrink, and Michael Hensel. “Intracellular Salmonella inhibit antigen presentation by dendritic cells.” The Journal of Immunology 174, no. 5 (2005): 2892-2899.

Chen, G. et al. Oral Delivery of the Sj23LHD-GST Antigen by Salmonella typhimurium Type III Secretion System Protects against Schistosoma japonicum Infection in Mice. PLoS Negl. Trop. Dis. 5, doi:10.1371/journal.pntd.0001313 (2011).

Chen, Inês, Theresa M. Finn, Liu Yanqing, Qi Guoming, Rino Rappuoli, and Mariagrazia Pizza. “A Recombinant Live Attenuated Strain of Vibrio cholerae Induces Immunity against Tetanus Toxin and Bordetella pertussis Tracheal Colonization Factor.” Infection and immunity 66, no. 4 (1998): 1648-1653.

Chen, X. et al. A Mouse Model of Clostridium difficile-Associated Disease. Gastroenterology 135, 1984-1992, doi:10.1053/j.gastro.2008.09.002 (2008).

Clairmont C, Lee K C, Pike J, Ittensohn M, Low K B, Pawelek J, Bermudes D, Brecher S M, Margitich D, Turnier J, Li Z, Luo X, King I, Zheng L M. 2000. Biodistribution and genetic stability of the novel antitumor agent VNP20009, a genetically modified strain of Salmonella typhimurium. The Journal of infectious diseases 181:1996-2002.

Clark-Curtiss, J. E. & Curtiss, R. Salmonella Vaccines: Conduits for Protective Antigens. Journal of immunology (Baltimore, Md.: 1950) 200, 39-48, doi:10.4049/jimmunol.1600608 (2018).

Cohen, O. R., Steele, J. A., Zhang, Q., Schmidt, D. J. & one, W.-Y. Systemically administered IgG anti-toxin antibodies protect the colonic mucosa during infection with Clostridium difficile in the piglet model. PLoS One, doi:10.1371/journal.pone.0111075 (2014).

Cote-Sierra, Javier, Erik Jongert, Amin Bredan, Dinesh C. Gautam, M. Parkhouse, Pierre Cornelis, Patrick De Baetselier, and Hilde Revets. “A new membrane-bound OprI lipoprotein expression vector: high production of heterologous fusion proteins in Gram (−) bacteria and the implications for oral vaccination.” Gene 221, no. 1 (1998): 25-34.

Darji, Ayub, Carlos A. Guzmán, Birgit Gerstel, Petra Wachholz, Kenneth N. Timmis, Jürgen Wehland, Trinad Chakraborty, and Siegfried Weiss. “Oral somatic transgene vaccination using attenuated S. typhimurium.” Cell 91, no. 6 (1997): 765-775.

Del Rio, Beatriz, Raymond J. Dattwyler, Miguel Aroso, Vera Neves, Luciana Meirelles, Jos F M L Seegers, and Maria Gomes-Solecki. “Oral immunization with recombinant Lactobacillus plantarum induces a protective immune response in mice with Lyme disease.” Clinical and Vaccine Immunology 15, no. 9 (2008): 1429-1435.

Detmer, Ann, and Jacob Glenting. “Live bacterial vaccines—a review and identification of potential hazards.” Microbial cell factories 5, no. 1 (2006): 23.

Donald, R. G. K. et al. A novel approach to generate a recombinant toxoid vaccine against Clostridium difficile. Microbiology 159, 1254-1266, doi:10.1099/mic.0.066712-0 (2013).

Du, Aifang, and Suhua Wang. “Efficacy of a DNA vaccine delivered in attenuated Salmonella typhimurium against Eimeria tenella infection in chickens.” International Journal for Parasitology 35, no. 7 (2005): 777-785.

Fayolle, C., D. O'Callaghan, P. Martineau, A. Charbit, J. M. Clément, M. Hofnung, and C. Leclerc. “Genetic control of antibody responses induced against an antigen delivered by recombinant attenuated Salmonella typhimurium.” Infection and immunity 62, no. 10 (1994): 4310-4319.

Freer G, Pistello M. 2018. Varicella-zoster virus infection: natural history, clinical manifestations, immunity and current and future vaccination strategies. New Microbiol 41:95-105.

Gahan, Michelle E., Diane E. Webster, Steven L. Wesselingh, and Richard A. Strugnell. “Impact of plasmid stability on oral DNA delivery by Salmonella enterica serovar Typhimurium.” Vaccine 25, no. 8 (2007): 1476-1483.

Galen, J. E., Buskirk, A. D., Tennant, S. M. & Pasetti, M. F. Live Attenuated Human Salmonella Vaccine Candidates: Tracking the Pathogen in Natural Infection and Stimulation of Host Immunity. EcoSal Plus 7, doi:10.1128/ecosalplus.ESP-0010-2016 (2016).

Garmory, Helen S., Sophie E C Leary, Kate F. Griffin, E. Diane Williamson, Katherine A. Brown, and Richard W. Titball. “The use of live attenuated bacteria as a delivery system for heterologous antigens.” Journal of drug targeting 11, no. 8-10 (2003): 471-479.

Gentschev, Ivaylo, Simone Spreng, Heike Sieber, Jose Ures, Fabian Mollet, Andre Collioud, Jon Pearman et al. “Vivotif®—a ‘magic shield’ for protection against typhoid fever and delivery of heterologous antigens.” Chemotherapy 53, no. 3 (2007): 177-180.

Georgiou, George, Christos Stathopoulos, Patrick S. Daugherty, Amiya R. Nayak, Brent L. Iverson, and Roy Curtiss III. “Display of heterologous proteins on the surface of microorganisms: from the screening of combinatorial libraries to live recombinant vaccines.” Nature biotechnology 15, no. 1 (1997): 29-34.

Gerding, Dale N. “Clostridium difficile infection prevention: biotherapeutics, immunologics, and vaccines.” Discovery medicine 13, no. 68 (2012): 75-83.

Gerlach, Roman G., and Michael Hensel. “Salmonella pathogenicity islands in host specificity, host pathogen-interactions and antibiotics resistance of Salmonella enterica.” Berliner und Munchener tierarztliche Wochenschrift 120, no. 7/8 (2007): 317.

Ghose, C. et al. Toll-like receptor 5-dependent immunogenicity and protective efficacy of a recombinant fusion protein vaccine containing the nontoxic domains of Clostridium difficile toxins A and B and Salmonella enterica serovar typhimurium flagellin in a mouse model of Clostridium difficile disease. Infect. Immun. 81, 2190-2196, doi:10.1128/IAI.01074-12 (2013).

Giannasca, Paul J., and Michel Warny. “Active and passive immunization against Clostridium difficile diarrhea and colitis.” Vaccine 22, no. 7 (2004): 848-856.

Gradoni, L. “An update on antileishmanial vaccine candidates and prospects for a canine Leishmania vaccine.” Veterinary parasitology 100, no. 1-2 (2001): 87-103.

Grangette, Corinne, Heide Müller-Alouf, Denise Goudercourt, Marie-Claude Geoffroy, Mireille Turneer, and Annick Mercenier. “Mucosal immune responses and protection against tetanus toxin after intranasal immunization with recombinant Lactobacillus plantarum.” Infection and immunity 69, no. 3 (2001): 1547-1553.

Grangette, Corinne, Heide Müller-Alouf, Denise Goudercourt, Marie-Claude Geoffroy, Mireille Turneer, and Annick Mercenier. “Mucosal immune responses and protection against tetanus toxin after intranasal immunization with recombinant Lactobacillus plantarum.” Infection and immunity 69, no. 3 (2001): 1547-1553.

Grangette, Corinne, Heide Müller-Alouf, Marie-Claude Geoffroy, Denise Goudercourt, Mireille Turneer, and Annick Mercenier. “Protection against tetanus toxin after intragastric administration of two recombinant lactic acid bacteria: impact of strain viability and in vivo persistence.” Vaccine 20, no. 27-28 (2002): 3304-3309.

Greenberg, R. N., Marbury, T. C., Foglia, G. & Warny, M. Phase I dose finding studies of an adjuvanted Clostridium difficile toxoid vaccine. Vaccine 30, 2245-2249, doi:10.1016/j.vaccine.2012.01.065 (2012).

Guo, Shanguang, Weiwei Yan, Sean P. McDonough, Nengfeng Lin, Katherine J. Wu, Hongxuan He, Hua Xiang, Maosheng Yang, Maira Aparecida S. Moreira, and Yung-Fu Chang. “The recombinant Lactococcus lactis oral vaccine induces protection against C. difficile spore challenge in a mouse model.” Vaccine 33, no. 13 (2015): 1586-1595, doi:10.1016/j.vaccine.2015.02.006.

Guzman, Carlos A., Stefan Borsutzky, Monika Griot-Wenk, Ian C. Metcalfe, Jon Pearman, Andre Collioud, Didier Favre, and Guido Dietrich. “Vaccines against typhoid fever.” Vaccine 24, no. 18 (2006): 3804-3811.

Hahn, Heinz P., and Bernd-Ulrich von Specht. “Secretory delivery of recombinant proteins in attenuated Salmonella strains: potential and limitations of Type I protein transporters.” FEMS Immunology & Medical Microbiology 37, no. 2-3 (2003): 87-98.

Hansson, Marianne, and Stefan Sta. “Design and production of recombinant subunit vaccines.” Biotechnology and applied biochemistry 32, no. 2 (2000): 95-107.

Harrison, J. A., B. Villarreal-Ramos, P. Mastroeni, R. Demarco de Hormaeche, and C. E. Hormaeche. “Correlates of protection induced by live Aro− Salmonella typhimurium vaccines in the murine typhoid model.” Immunology 90, no. 4 (1997): 618-625.

Haselbeck, A. H. et al. Current perspectives on invasive nontyphoidal Salmonella disease. Curr. Opin. Infect. Dis. 30, 498-503, doi:10.1097/QCO.0000000000000398 (2017).

Hayashi, F. et al. The innate immune response to bacterial flagellin is mediated by Toll-like receptor 5. Nature 410, 1099-1103, doi:10.1038/35074106 (2001).

Heimann, S. M., Cruz Aguilar, M. R., Mellinghof, S. & Vehreschild, M. J. G. T. J. Economic burden and cost-effective management of Clostridium difficile infections. Med. Mal. Infect. 48, 23-29, doi:10.1016/j.medmal.2017.10.010 (2018).

Hindle, Z. et al. Characterization of Salmonella enterica derivatives harboring defined aroC and Salmonella pathogenicity island 2 type III secretion system (ssaV) mutations by immunization of healthy volunteers. Infect. Immun. 70, 3457-3467 (2002).

Hong, Huynh A., Krisztina Hitri, Siamand Hosseini, Natalia Kotowicz, Donna Bryan, Fatme Mawas, Anthony J. Wilkinson, Annie van Broekhoven, Jonathan Kearsey, and Simon M. Cutting. “Mucosal antibodies to the C-terminus of toxin A prevent colonization of Clostridium difficile.” Infection and immunity (2017): IAI-01060. doi:10.1128/IAI.01060-16.

Huang, Jen-Min, Michela Sali, Matthew W. Leckenby, David S. Radford, Hong A. Huynh, Giovanni Delogu, Rocky M. Cranenburgh, and Simon M. Cutting. “Oral delivery of a DNA vaccine against tuberculosis using operator-repressor titration in a Salmonella enterica vector.” Vaccine 28, no. 47 (2010): 7523-7528.

Husseiny, Mohamed I., and Michael Hensel. “Evaluation of an intracellular-activated promoter for the generation of live Salmonella recombinant vaccines.” Vaccine 23, no. 20 (2005): 2580-2590.

Ibarra, A. J. & Mortimer, O. Salmonella—the ultimate insider. Salmonella virulence factors that modulate intracellular survival. Cell. Microbiol. 11, 1579-1586, doi:10.1111/j.1462-5822.2009.01368.x (2009).

Jabbar, Ibtissam A., Germain J P Fernando, Nick Saunders, Anne Aldovini, Richard Young, Karen Malcolm, and Ian H. Frazer. “Immune responses induced by BCG recombinant for human papillomavirus L1 and E7 proteins.” Vaccine 18, no. 22 (2000): 2444-2453.

Jensen, Eric R., Hao Shen, Felix O. Wettstein, Rafi Ahmed, and Jeff F. Miller. “Recombinant Listeria monocytogenes as a live vaccine vehicle and a probe for studying cell-mediated immunity.” Immunological reviews 158, no. 1 (1997): 147-157.

Jepson, M. A. & and infection, C.-M. A. The role of M cells in Salmonella infection. Microbes and infection (2001).

Kardani, K., Bolhassani, A. & Vaccine, S.-S. Prime-boost vaccine strategy against viral infections: Mechanisms and benefits. Vaccine (2016).

Karsten, Verena, Sean R. Murray, Jeremy Pike, Kimberly Troy, Martina Ittensohn, Manvel Kondradzhyan, K. Brooks Low, and David Bermudes. “msbB deletion confers acute sensitivity to CO 2 in Salmonella enterica serovar Typhimurium that can be suppressed by a loss-of-function mutation in zwf.” BMC microbiology 9, no. 1 (2009): 170.

Kelly, Ciaran P., and Lorraine Kyne. “The host immune response to Clostridium difficile.” Journal of medical microbiology 60, no. 8 (2011): 1070-1079.

Killeen, K., D. Spriggs, and J. Mekalanos. “Bacterial mucosal vaccines: Vibrio cholerae as a live attenuated vaccine/vector paradigm.” In Defense of Mucosal Surfaces: Pathogenesis, Immunity and Vaccines, pp. 237-254. Springer, Berlin, Heidelberg, 1999.

Kim, K. S., M. C. Jenkins, and HYUN S. Lillehoj. “Immunization of chickens with live Escherichia coli expressing Eimeria acervulina merozoite recombinant antigen induces partial protection against coccidiosis.” Infection and immunity 57, no. 8 (1989): 2434-2440.

Knudsen, Maria Lisa, Karl Ljungberg, Maria Kakoulidou, Linda Kostic, David Hallengärd, Juan García-Arriaza, Andres Merits, Mariano Esteban, and Peter Liljeström. “Kinetic and phenotypic analysis of CD8+ T cell responses after priming with alphavirus replicons and homologous or heterologous booster immunizations.” Journal of virology (2014): JVI-02223. doi:10.1128/JVI.02223-14.

Kociolek, L. K. & Gerding, D. N. Breakthroughs in the treatment and prevention of Clostridium difficile infection. Nature reviews. Gastroenterology & hepatology, doi:10.1038/nrgastro.2015.220 (2016).

Kotton, Camille N., and Elizabeth L. Hohmann. “Enteric pathogens as vaccine vectors for foreign antigen delivery.” Infection and immunity 72, no. 10 (2004): 5535-5547.

Kotton, Camille N., and Elizabeth L. Hohmann. “Enteric pathogens as vaccine vectors for foreign antigen delivery.” Infection and immunity 72, no. 10 (2004): 5535-5547.

Kuehne, S. A., Cartman, S. T., Heap, J. T. & Nature, K.-M. L. The role of toxin A and toxin B in Clostridium difficile infection. Nature (2010).

Kuipers, Kirsten, Maria H. Daleke-Schermerhorn, Wouter S P Jong, M. Corinne, Fred van Opzeeland, Elles Simonetti, Joen Luirink, and Marien I. de Jonge. “Salmonella outer membrane vesicles displaying high densities of pneumococcal antigen at the surface offer protection against colonization.” Vaccine 33, no. 17 (2015): 2022-2029.

Lakhashe, S. K., Byrareddy, S. N., Zhou, M. & Vaccine, B.-B. C. Multimodality vaccination against clade C SHIV: partial protection against mucosal challenges with a heterologous tier 2 virus. Vaccine (2014).

Lee, Bruce Y., Michael J. Popovich, Ye Tian, Rachel R. Bailey, Paul J. Ufberg, Ann E. Wiringa, and Robert R. Muder. “The potential value of Clostridium difficile vaccine: an economic computer simulation model.” Vaccine 28, no. 32 (2010): 5245-5253.

Lee, Jong-Soo, Kwang-Soon Shin, Jae-Gu Pan, and Chul-Joong Kim. “Surface-displayed viral antigens on Salmonella carrier vaccine.” Nature biotechnology 18, no. 6 (2000): 645.

Li, Long, Weihuan Fang, Jianrong Li, Li Fang, Yaowei Huang, and Lian Yu. “Oral DNA vaccination with the polyprotein gene of infectious bursal disease virus (IBDV) delivered by attenuated Salmonella elicits protective immune responses in chickens.” Vaccine 24, no. 33-34 (2006): 5919-5927.

Liljeqvist, Sissela, and Stefan Ståhl. “Production of recombinant subunit vaccines: protein immunogens, live delivery systems and nucleic acid vaccines.” Journal of biotechnology 73, no. 1 (1999): 1-33.

Loessner, Holger, and Siegfried Weiss. “Bacteria-mediated DNA transfer in gene therapy and vaccination.” Expert opinion on biological therapy 4, no. 2 (2004): 157-168.

Loessner, Holger, Anne Endmann, Sara Leschner, Heike Bauer, Andrea Zelmer, Susanne zur Lage, Kathrin Westphal, and Siegfried Weiss. “Improving live attenuated bacterial carriers for vaccination and therapy.” International Journal of Medical Microbiology 298, no. 1-2 (2008): 21-26.

Luke, C. J. & review of vaccines, S.-K. Improving pandemic H5N1 influenza vaccines by combining different vaccine platforms. Expert review of vaccines, doi:10.1586/14760584.2014.922416 (2014).

Makvandi, Manoochehr, Ali Teimoori, Mehdi Parsa Nahad, Ali Khodadadi, and Milad Zandi. “Expression of Salmonella typhimurium and Escherichia coli flagellin protein and its functional characterization as an adjuvant.” Microbial pathogenesis 118 (2018): 87-90.

McSorley, Stephen J., Damo Xu, and FYs Liew. “Vaccine efficacy of Salmonella strains expressing glycoprotein 63 with different promoters.” Infection and immunity 65, no. 1 (1997): 171-178.

Metzger, Wolfram G., E. Mansouri, M. Kronawitter, Susanne Diescher, Meike Soerensen, Robert Hurwitz, Dirk Bumann, Toni Aebischer, B-U. Von Specht, and Thomas F. Meyer. “Impact of vector-priming on the immunogenicity of a live recombinant Salmonella enterica serovar typhi Ty21a vaccine expressing urease A and B from Helicobacter pylori in human volunteers.” Vaccine 22, no. 17-18 (2004): 2273-2277.

Mielcarek, Nathalie, Sylvie Alonso, and Camille Locht. “Nasal vaccination using live bacterial vectors.” Advanced drug delivery reviews 51, no. 1-3 (2001): 55-69.

Mohamadzadeh, Mansour, Tri Duong, Timothy Hoover, and Todd R. Klaenhammer. “Targeting mucosal dendritic cells with microbial antigens from probiotic lactic acid bacteria.” Expert review of vaccines 7, no. 2 (2008): 163-174.

Nagarajan, Arvindhan G., Sudhagar V. Balasundaram, Jessin Janice, Guruswamy Karnam, Sandeepa M. Eswarappa, and Dipshikha Chakravortty. “SopB of Salmonella enterica serovar Typhimurium is a potential DNA vaccine candidate in conjugation with live attenuated bacteria.” Vaccine 27, no. 21 (2009): 2804-2811.

Niedergang, Florence, Jean-Claude Sirard, Corinne Tallichet Blanc, and Jean-Pierre Kraehenbuhl. “Entry and survival of Salmonella typhimurium in dendritic cells and presentation of recombinant antigens do not require macrophage-specific virulence factors.” Proceedings of the National Academy of Sciences 97, no. 26 (2000): 14650-14655.

Oggioni, Marco R., Riccardo Manganelli, Mario Contorni, Massimo Tommasino, and Gianni Pozzi. “Immunization of mice by oral colonization with live recombinant commensal streptococci.” Vaccine 13, no. 8 (1995): 775-779.

Okan, Nihal A., Patricio Mena, Jorge L. Benach, James B. Bliska, and A. Wali Karzai. “The smpB-ssrA mutant of Yersinia pestis functions as a live attenuated vaccine to protect mice against pulmonary plague infection.” Infection and immunity 78, no. 3 (2010): 1284-1293.

Pace, John Lee, Richard Ives Walker, and Steven Michael Frey. “Methods for producing enhanced antigenic Campylobacter bacteria and vaccines.” U.S. Pat. No. 5,679,564, issued Oct. 21, 1997.

Paglia, Paola, Ivano Arioli, Nicole Frahm, Trinad Chakraborty, Mario P. Colombo, and Carlos A. Guzmàn. “The defined attenuated Listeria monocytogenes Δmpl2 mutant is an effective oral vaccine carrier to trigger a long-lasting immune response against a mouse fibrosarcoma.” European journal of immunology 27, no. 6 (1997): 1570-1575.

Panthel, K., Meinel, K. M., Sevil Domènech, V. E. E., Trülzsch, K. & Rüssmann, H. Salmonella type III-mediated heterologous antigen delivery: a versatile oral vaccination strategy to induce cellular immunity against infectious agents and tumors. International journal of medical microbiology: IJMM 298, 99-103, doi:10.1016/j.ijmm.2007.07.002 (2008).

Pasetti, Marcela F., Myron M. Levine, and Marcelo B. Sztein. “Animal models paving the way for clinical trials of attenuated Salmonella enterica serovar Typhi live oral vaccines and live vectors.” Vaccine 21, no. 5-6 (2003): 401-418.

Paterson, Yvonne, Patrick D. Guirnalda, and Laurence M. Wood. “Listeria and Salmonella bacterial vectors of tumor-associated antigens for cancer immunotherapy.” In Seminars in immunology, vol. 22, no. 3, pp. 183-189. Academic Press, 2010.

Paterson, Yvonne. “Specific immunotherapy of cancer using a live recombinant bacterial vaccine vector.” U.S. Pat. No. 6,051,237, issued Apr. 18, 2000.

Patyar, S., R. Joshi, D S Prasad Byrav, A. Prakash, B. Medhi, and B. K. Das. “Bacteria in cancer therapy: a novel experimental strategy.” Journal of biomedical science 17, no. 1 (2010): 21.

Pawelek, John M., K. Brooks Low, and David Bermudes. “Tumor-targeted Salmonella as a novel anticancer vector.” Cancer research 57, no. 20 (1997): 4537-4544.

Péchiné, Séverine, Cécile Denéve, Alban Le Monnier, Sandra Hoys, Claire Janoir, and Anne Collignon. “Immunization of hamsters against Clostridium difficile infection using the Cwp84 protease as an antigen.” FEMS Immunology & Medical Microbiology 63, no. 1 (2011): 73-81.

Péchiné, Séverine, Claire Janoir, Hélène Boureau, Aude Gleizes, Nicolas Tsapis, Sandra Hoys, Elias Fattal, and Anne Collignon. “Diminished intestinal colonization by Clostridium difficile and immune response in mice after mucosal immunization with surface proteins of Clostridium difficile.” Vaccine 25, no. 20 (2007): 3946-3954.

Penha Filho, R. A. et al. Humoral and cellular immune response generated by different vaccine programs before and after Salmonella Enteritidis challenge in chickens. Vaccine 30, 7637-7643, doi:10.1016/j.vaccine.2012.10.020 (2012).

Prisco, A. & concepts, D. P. Memory immune response: a major challenge in vaccination. Biomolecular concepts (2012).

Qu, Daofeng, Suhua Wang, Weiming Cai, and Aifang Du. “Protective effect of a DNA vaccine delivered in attenuated Salmonella typhimurium against Toxoplasma gondii infection in mice.” Vaccine 26, no. 35 (2008): 4541-4548.

Reigadas, E., L. Alcala, M. Marín, A. Martin, C. Iglesias, and E. Bouza. “Role of binary toxin in the outcome of Clostridium difficile infection in a non-027 ribotype setting.” Epidemiology & Infection 144, no. 2 (2016): 268-273. doi:10.1017/s095026881500148x (2015).

Reveneau, Nathalie, Marie-Claude Geoffroy, Camille Locht, Patrice Chagnaud, and Annick Mercenier. “Comparison of the immune responses induced by local immunizations with recombinant Lactobacillus plantarum producing tetanus toxin fragment C in different cellular locations.” Vaccine 20, no. 13-14 (2002): 1769-1777.

Robinson, Karen, Lisa M. Chamberlain, Karin M. Schofield, Jeremy M. Wells, and Richard W F Le Page. “Oral vaccination of mice against tetanus with recombinant Lactococcus lactis.” Nature biotechnology 15, no. 7 (1997): 653.

Rosenkranz, Claudia D., Damasia Chiara, Caroline Agorio, Adriana Baz, Marcela F. Pasetti, Fernanda Schreiber, Silvia Dematteis, Miguel Martinez, Marcelo B. Sztein, and Jose A. Chabalgoity. “Towards new immunotherapies: targeting recombinant cytokines to the immune system using live attenuated Salmonella.” Vaccine 21, no. 7-8 (2003): 798-801.

Ross, Bruce C., Larissa Czajkowski, Dianna Hocking, Mai Margetts, Elizabeth Webb, Linda Rothel, Michelle Patterson et al. “Identification of vaccine candidate antigens from a genomic analysis of Porphyromonas gingivalis.” Vaccine 19, no. 30 (2001): 4135-4142.

Rota P A, Khan A S, Durigon E, Yuran T, Villamarzo Y S, Bellini W J. 1995. Detection of measles virus RNA in urine specimens from vaccine recipients. J Clin Microbiol 33:2485-2488.

Rupnik, M., Wilcox, M. H. & Microbiology, G. D. N. Clostridium difficile infection: new developments in epidemiology and pathogenesis. Clostridium difficile infection: new developments in epidemiology and pathogenesis, doi:10.1038/nrmicro2164 (2009).

Ryan, Edward T., Joan R. Butterton, Rex Neal Smith, Patricia A. Carroll, Thomas I. Crean, and Stephen B. Calderwood. “Protective immunity against Clostridium difficile toxin A induced by oral immunization with a live, attenuated Vibrio cholerae vector strain.” Infection and immunity 65, no. 7 (1997): 2941-2949.

Santos, Renato L., Shuping Zhang, Renée M. Tsolis, Robert A. Kingsley, L. Garry Adams, and Andreas J. Bäumler. “Animal models of Salmonella infections: enteritis versus typhoid fever.” Microbes and Infection 3, no. 14-15 (2001): 1335-1344.

Sbrogio-Almeida, M. E., Tainá Mosca, L. M. Massis, I. A. Abrahamsohn, and L. C. S. Ferreira. “Host and bacterial factors affecting induction of immune responses to flagellin expressed by attenuated Salmonella vaccine strains.” Infection and immunity 72, no. 5 (2004): 2546-2555.

Schorr, Joachim, Bernhard Knapp, Erika Hundt, Hans A. Küpper, and Egon Amann. “Surface expression of malarial antigens in Salmonella typhimurium: induction of serum antibody response upon oral vaccination of mice.” Vaccine 9, no. 9 (1991): 675-681.

Seegers, Jos F M L. “Lactobacilli as live vaccine delivery vectors: progress and prospects.” Trends in biotechnology 20, no. 12 (2002): 508-515.

Shams, Homayoun, Fernando Poblete, Holger Rüssmann, Jorge E. Galán, and Ruben O. Donis. “Induction of specific CD8+ memory T cells and long lasting protection following immunization with Salmonella typhimurium expressing a lymphocytic choriomeningitis MHC class I-restricted epitope.” Vaccine 20, no. 3-4 (2001): 577-585.

Shen, Hao, Mark K. Slifka, Mehrdad Matloubian, Eric R. Jensen, Rafi Ahmed, and Jeff F. Miller. “Recombinant Listeria monocytogenes as a live vaccine vehicle for the induction of protective anti-viral cell-mediated immunity.” Proceedings of the National Academy of Sciences 92, no. 9 (1995): 3987-3991.

Shukarev, Georgi, Benoit Callendret, Kerstin Luhn, Macaya Douoguih, and EBOVAC1 consortium. “A two-dose heterologous prime-boost vaccine regimen eliciting sustained immune responses to Ebola Zaire could support a preventive strategy for future outbreaks.” Human vaccines & immunotherapeutics 13, no. 2 (2017): 266-270. doi:10.1080/21645515.2017.1264755.

Silin, Dmytro S., Oksana V. Lyubomska, Vichai Jirathitikal, and Aldar S. Bourinbaiar. “Oral vaccination: where we are?.” Expert opinion on drug delivery 4, no. 4 (2007): 323-340.

Silva, Adilson José da, Teresa Cristina Zangirolami, Maria Teresa Marques Novo-Mansur, Roberto de Campos Giordano, and Elizabeth Angélica Leme Martins. “Live bacterial vaccine vectors: an overview.” Brazilian Journal of Microbiology 45, no. 4 (2014): 1117-1129.

Sjöstedt, A., G. Sandström, and A. Tärnvik. “Humoral and cell-mediated immunity in mice to a 17-kilodalton lipoprotein of Francisella tularensis expressed by Salmonella typhimurium.” Infection and immunity 60, no. 7 (1992): 2855-2862.

Solanki, Amit Kumar, Bharati Bhatia, Himani Kaushik, Sachin K. Deshmukh, Aparna Dixit, and Lalit C. Garg. “Clostridium perfringens beta toxin DNA prime-protein boost elicits enhanced protective immune response in mice.” Applied microbiology and biotechnology 101, no. 14 (2017): 5699-5708. doi:10.1007/s00253-017-8333-2.

Spreng, Simone, Guido Dietrich, and Gerald Weidinger. “Rational design of Salmonella-based vaccination strategies.” Methods 38, no. 2 (2006): 133-143.

Srinivasan, Aparna, Joseph Foley, and Stephen J. McSorley. “Massive number of antigen-specific CD4 T cells during vaccination with live attenuated Salmonella causes interclonal competition.” The Journal of Immunology 172, no. 11 (2004): 6884-6893.

Ståhl, Stefan, and Mathias Uhlén. “Bacterial surface display: trends and progress.” Trends in biotechnology 15, no. 5 (1997): 185-192.

Stevenson, Gordon, and Paul A. Manning. “Galactose epimeraseless (GalE) mutant G30 of Salmonella typhimurium is a good potential live oral vaccine carrier for fimbrial antigens.” FEMS microbiology letters 28, no. 3 (1985): 317-321.

Stocker, Bruce A D, and Salete M C Newton. “Immune responses to epitopes inserted in Salmonella flagellin.” International reviews of immunology 11, no. 2 (1994): 167-178.

Stocker, Bruce A D. “Novel non-reverting Salmonella live vaccines.” U.S. Pat. No. 4,735,801, issued Apr. 5, 1988.

Strindelius, Lena, Malin Filler, and Ingvar Sjöholm. “Mucosal immunization with purified flagellin from Salmonella induces systemic and mucosal immune responses in C3H/HeJ mice.” Vaccine 22, no. 27-28 (2004): 3797-3808.

Surawicz, C. M. & Alexander, J. Treatment of refractory and recurrent Clostridium difficile infection. Nature Reviews Gastroenterology and Hepatology 8, doi:10.1038/nrgastro.2011.59 (2011).

Takata, Tetsuo, Toshiro Shirakawa, Yoshiko Kawasaki, Shohiro Kinoshita, Akinobu Gotoh, Yasunobu Kano, and Masato Kawabata. “Genetically engineered Bifidobacterium animalis expressing the Salmonella flagellin gene for the mucosal immunization in a mouse model.” The Journal of Gene Medicine: A cross-disciplinary journal for research on the science of gene transfer and its clinical applications 8, no. 11 (2006): 1341-1346.

Thatte, Jayant, Satyajit Rath, and Vineeta Bal. “Immunization with live versus killed Salmonella typhimurium leads to the generation of an IFN-γ-dominant versus an IL-4-dominant immune response.” International immunology 5, no. 11 (1993): 1431-1436.

Tian, J.-H. H. et al. A novel fusion protein containing the receptor binding domains of C. difficile toxin A and toxin B elicits protective immunity against lethal toxin and spore challenge in preclinical efficacy models. Vaccine 30, 4249-4258, doi:10.1016/j.vaccine.2012.04.045 (2012).

Tite, J. P., X. M. Gao, C. M. Hughes-Jenkins, M. Lipscombe, D. O'Callaghan, G. Dougan, and F. Y. Liew. “Anti-viral immunity induced by recombinant nucleoprotein of influenza A virus. III. Delivery of recombinant nucleoprotein to the immune system using attenuated Salmonella typhimurium as a live carrier.” Immunology 70, no. 4 (1990): 540.

Toso J F, Gill V J, Hwu P, Marincola F M, Restifo N P, Schwartzentruber D J, Sherry R M, Topalian S L, Yang J C, Stock F, Freezer L J, Morton K E, Seipp C, Haworth L, Mavroukakis S, White D, MacDonald S, Mao J, Sznol M, Rosenberg S A. 2002. Phase I study of the intravenous administration of attenuated Salmonella typhimurium to patients with metastatic melanoma. Journal of clinical oncology: official journal of the American Society of Clinical Oncology 20:142-152.

Toussaint, Bertrand, Xavier Chauchet, Yan Wang, Benoit Polack, and Audrey Le Gouëllec. “Live-attenuated bacteria as a cancer vaccine vector.” Expert review of vaccines 12, no. 10 (2013): 1139-1154.

Troy S B, Ferreyra-Reyes L, Huang C, Sarnquist C, Canizales-Quintero S, Nelson C, Baez, Saldana R, Holubar M, Ferreira-Guerrero E, Garcia-Garcia L, Maldonado Y A. 2014. Community circulation patterns of oral polio vaccine serotypes 1, 2, and 3 after Mexican national immunization weeks. J Infect Dis 209:1693-1699.

Van Immerseel, Filip, U. Methner, I. Rychlik, B. Nagy, P. Velge, G. Martin, N. Foster, Richard Ducatelle, and Paul A. Barrow. “Vaccination and early protection against non-host-specific Salmonella serotypes in poultry: exploitation of innate immunity and microbial activity.” Epidemiology & Infection 133, no. 6 (2005): 959-978.

van Kleef, E., Deeny, S. R., Jit, M. & Vaccine, C.-B. The projected effectiveness of Clostridium difficile vaccination as part of an integrated infection control strategy. Vaccine (2016).

Walker, Mark J., Manfred Rohde, Kenneth N. Timmis, and Carlos A. Guzman. “Specific lung mucosal and systemic immune responses after oral immunization of mice with Salmonella typhimurium aroA, Salmonella typhi Ty21a, and invasive Escherichia coli expressing recombinant pertussis toxin S1 subunit.” Infection and immunity 60, no. 10 (1992): 4260-4268.

Wang, Shifeng, Qingke Kong, and Roy Curtiss III. “New technologies in developing recombinant attenuated Salmonella vaccine vectors.” Microbial pathogenesis 58 (2013): 17-28.

Wang, Shifeng, Yuhua Li, Huoying Shi, Wei Sun, Kenneth L. Roland, and Roy Curtiss. “Comparison of a regulated delayed antigen synthesis system with in vivo-inducible promoters for antigen delivery by live attenuated Salmonella vaccines.” Infection and immunity 79, no. 2 (2011): 937-949.

Ward, Stephen J., Gill Douce, Dayse Figueiredo, Gordon Dougan, and Brendan W. Wren. “Immunogenicity of a Salmonella typhimurium aroA aroD vaccine expressing a nontoxic domain of Clostridium difficile toxin A.” Infection and immunity 67, no. 5 (1999): 2145-2152.

Warren, C. A. et al. Amixicile, a novel inhibitor of pyruvate: ferredoxin oxidoreductase, shows efficacy against Clostridium difficile in a mouse infection model. Antimicrob. Agents Chemother. 56, 4103-4111, doi:10.1128/AAC.00360-12 (2012).

Wells, J. M., K. Robinson, L. M. Chamberlain, K. M. Schofield, and R. W. F. Le Page. “Lactic acid bacteria as vaccine delivery vehicles.” Antonie Van Leeuwenhoek 70, no. 2-4 (1996): 317-330.

Wiegand, P. N. et al. Clinical and economic burden of Clostridium difficile infection in Europe: a systematic review of healthcare-facility-acquired infection. J. Hosp. Infect. 81, 1-14, doi:10.1016/j.jhin.2012.02.004 (2012).

Wilcox, Mark H., Dale N. Gerding, Ian R. Poxton, Ciaran Kelly, Richard Nathan, Thomas Birch, Oliver A. Cornely et al. “Bezlotoxumab for prevention of recurrent Clostridium difficile infection.” New England Journal of Medicine 376, no. 4 (2017): 305-317. doi:10.1056/NEJMoa1602615.

Wu, Jane Y., Salete Newton, Amrit Judd, Bruce Stocker, and William S. Robinson. “Expression of immunogenic epitopes of hepatitis B surface antigen with hybrid flagellin proteins by a vaccine strain of Salmonella.” Proceedings of the National Academy of Sciences 86, no. 12 (1989): 4726-4730.

Wyszyńska, Agnieszka, Patrycja Kobierecka, Jacek Bardowski, and Elżbieta Katarzyna Jagusztyn-Krynicka. “Lactic acid bacteria—20 years exploring their potential as live vectors for mucosal vaccination.” Applied microbiology and biotechnology 99, no. 7 (2015): 2967-2977.

Xiong, G. et al. Novel cancer vaccine based on genes of Salmonella pathogenicity island 2. Int. J. Cancer 126, 2622-2634, doi:10.1002/ijc.24957 (2010).

Xu, Fengfeng, Mei Hong, and Jeffrey B. Ulmer. “Immunogenicity of an HIV-1 gag DNA vaccine carried by attenuated Shigella.” Vaccine 21, no. 7-8 (2003): 644-648.

Xu, Yigang, and Yijing Li. “Induction of immune responses in mice after intragastric administration of Lactobacillus casei producing porcine parvovirus VP2 protein.” Applied and environmental microbiology 73, no. 21 (2007): 7041-7047.

Yen C, Jakob K, Esona M D, Peckham X, Rausch J, Hull J J, Whittier S, Gentsch J R, LaRussa P. 2011. Detection of fecal shedding of rotavirus vaccine in infants following their first dose of pentavalent rotavirus vaccine. Vaccine 29:4151-4155.

Zegers, N. D., E. Kluter, H. van Der Stap, E. Van Dura, P. Van Dalen, M. Shaw, and L. Baillie. “Expression of the protective antigen of Bacillus anthracis by Lactobacillus casei: towards the development of an oral vaccine against anthrax.” Journal of applied microbiology 87, no. 2 (1999): 309-314.

Zhang, Ling, Lifang Gao, Lijuan Zhao, Baofeng Guo, Kun Ji, Yong Tian, Jinguo Wang et al. “Intratumoral delivery and suppression of prostate tumor growth by attenuated Salmonella enterica serovar typhimurium carrying plasmid-based small interfering RNAs.” Cancer research 67, no. 12 (2007): 5859-5864.

Zhang, Shanshan, Sarah Palazuelos-Munoz, Evelyn M. Balsells, Harish Nair, Ayman Chit, and Moe H. Kyaw. “Cost of hospital management of Clostridium difficile infection in United States—a meta-analysis and modelling study.” BMC infectious diseases 16, no. 1 (2016): 447.

Zhao, Zhanqin, Yun Xue, Bin Wu, Xibiao Tang, Ruiming Hu, Yindi Xu, Aizhen Guo, and Huanchun Chen. “Subcutaneous vaccination with attenuated Salmonella enterica serovar Choleraesuis C500 expressing recombinant filamentous hemagglutinin and pertactin antigens protects mice against fatal infections with both S. enterica serovar Choleraesuis and Bordetella bronchiseptica.” Infection and immunity 76, no. 5 (2008): 2157-2163.

Recently developed approaches to delivery of therapeutic molecules (U.S. Pat. Nos. 8,241,623; 8,524,220; 8,771,669; and 8,524,220) have coupled a protease sensitive therapeutic molecule with co-expression of protease inhibitors, expressly incorporated by reference herein.

Use of secreted proteins in live bacterial vectors has been demonstrated by several authors. Holland et al. (U.S. Pat. No. 5,143,830) have illustrated the use of fusions with the C-terminal portion of the hemolysin A (hlyA) gene, a member of the type I secretion system. When co-expressed in the presence of the hemolysin protein secretion channel (hlyBD) and a functional TolC, heterologous fusions are readily secreted from the bacteria. The type I secretion system that has been utilized most widely, and although it is currently considered the best system available, is thought to have limitations for delivery by attenuated bacteria (Hahn and Specht, 2003, FEMS Immunology and Medical Microbiology, 37: 87-98). Those limitations include the amount of protein secreted and the ability of the protein fused to it to interfere with secretion. Improvements of the type I secretion system have been demonstrated by Sugamata and Shiba (2005 Applied and Environmental Microbiology 71: 656-662) using a modified hlyB, and by Gupta and Lee (2008 Biotechnology and Bioengineering, 101: 967-974) by addition of rare codons to the hlyA gene, each of which is expressly incorporated by reference in their entirety herein. Fusion to the gene ClyA (Galen et al., 2004, Infection and Immunity, 72: 7096-7106 and Type III secretion proteins have also been used. Surface display has been used to export proteins outside of the bacteria. For example, fusion of the Lpp protein amino acids 1-9 with the transmembrane region B3-B7 of OmpA has been used for surface display (Samuelson et al., 2002, Display of proteins on bacteria, J. Biotechnology 96: 129-154, expressly incorporated by reference in its entirety herein).

The autotransporter surface display has been described by Berthet et al., WO/2002/070645, expressly incorporated by reference herein. Other heterologous protein secretion systems utilizing the autotransporter family can be modulated to result in either surface display or complete release into the medium (see Henderson et al., 2004, Type V secretion pathway: the autotransporter story, Microbiology and Molecular Biology Reviews 68: 692-744; Jose, 2006 Applied Microbiol. Biotechnol. 69: 607-614; Jose J, Zangen D (2005) Autodisplay of the protease inhibitor aprotinin in Escherichia coli. Biochem Biophys Res Commun 333:1218-1226 and Rutherford and Mourez 2006 Microbial Cell Factories 5: 22). For example, Veiga et al. (2003 Journal of Bacteriology 185: 5585-5590 and Klauser et al., 1990 EMBO Journal 9: 1991-1999) demonstrated hybrid proteins containing the β-autotransporter domain of the immunoglobulin A (IgA) protease of Nisseria gonorrhea. Fusions to flagellar proteins have been demonstrated. The peptide, usually of 15 to 36 amino acids in length, is inserted into the central, hypervariable region of the FliC gene such as that from Salmonella muenchen (Verma et al. 1995 Vaccine 13: 235-24; Wu et al., 1989 Proc. Natl. Acad. Sci. USA 86: 4726-4730; Cuadro et al., 2004 Infect. Immun. 72: 2810-2816; Newton et al., 1995, Res. Microbiol. 146: 203-216, expressly incorporated by reference in their entirety herein). Multihybrid FliC insertions of up to 302 amino acids have also been prepared (Tanskanen et al. 2000, Appl. Env. Microbiol. 66: 4152-4156, expressly incorporated by reference in its entirety herein).

Trimerization of antigens can be achieved using the T4 fibritin foldon trimerization sequence (Wei et al. 2008 J. Virology 82: 6200-6208) and VASP tetramerization domains (Kühnel et al., 2004 PNAS 101: 17027-17032), expressly incorporated by reference in their entirety herein. The multimerization domains are used to create, bi-specific, tri-specific, and quatra-specific targeting agents, whereby each individual agent is expressed with a multimerization tag, each of which may have the same or separate targeting peptide, such that following expression, surface display, secretion and/or release, they form multimers with multiple targeting domains. A fusion with the Pseudomonas ice nucleation protein (INP) wherein the N- and C-terminus of INP with an internal deletion consisting of the first 308 amino acids is followed by the mature sequence of the protein to be displayed (Jung et al., 1998, Surface display of Zymomonas mobilis levansucrase by using ice-nucleation protein of Pseudomonas syringae, Nature Biotechnology 16: 576-580; Kim et al., 2000, Bacterial surface display of an enzyme library for selective screening of improved cellulase variants, Applied and Environmental Microbiology 66: 788-793; Part:BBa_K811003 from www.iGEM.org; WO2005005630).

Salmonella are also encompassed that are, for example, attenuated in virulence by mutations in a variety of metabolic and structural genes. The technology therefore may provide a live composition for treating cancer comprising a live attenuated bacterium that is a serovar of Salmonella enterica comprising an attenuating mutation in a genetic locus of the chromosome of said bacterium that attenuates virulence of said bacterium and wherein said attenuating mutation is a combinations of other known attenuating mutations. Other attenuating mutation useful in the Salmonella bacterial strains described herein may be in a genetic locus selected from the group consisting of phoP, phoQ, edt, cya, crp, poxA, rpoS, htrA, nuoG, pmi, pabA, pts, damA, met, cys, pur, purA, purB, purI, purF, leu, ilv, arg, lys, zwf, aroA, aroB, aroC, aroD, serC, gua, cadA, rfc, rjb, rfa, ompR, msbB, pfkAB, crr, glk, ptsG, ptsHI, manXYZ and combinations thereof. The strain may also contain a mutation known as “Suwwan”, which is an approximately 100 kB deletion between two IS200 elements. The strain may also carry a defective thioredoxin gene (trxA−; which may be used i□combi□atio□with a TrxA fusio□), a defective glutathio□e oxidoreductase (gor−) and optionally, overexpress a protein disulfide bond isomerase (DsbA). The strain may also be engineered to express invasion and/or escape genes tlyA, tlyC patI and pld from Rickettsia, whereby the bacteria exhibit enhanced invasion and/or escape from the phagolysosome (Witworth et al., 2005, Infect. Immun. 73:6668-6673), thereby enhancing the activity of the effector genes described below. The strain may also be engineered to be deleted in an avirulence (anti-virulence) gene, such as zirTS, grvA and/or pcgL, or express the E. coli lac repressor, which is also an avirulence gene in order to compensate for over-attenuation. The strain may also express SlyA, a known transcriptional activator. In a preferred embodiment, the Salmonella strains are msbB mutants (msbB⁻). In a more preferred embodiment, the strains are msbB− and Suwwan. In a more preferred embodiment the strains are msbB⁻, Suwwan and zwf⁻. Zwf has recently been shown to provide resistance to CO2, acidic pH and osmolarity (Karsten et al., 2009, BMC Microbiology August 18; 9:170). Use of the msbB zwf genetic combination is also particularly preferred for use in combination with administered carbogen (an oxygen carbon dioxide mixture that may enhance delivery of therapeutic agents to a tumor). In a more preferred embodiment, the strains are msbB⁻, Suwwan, zwf⁻ and trxA⁻. In a most preferred embodiment, the strains are msbB⁻, Suwwan, zwf⁻, trxA⁻ and gor⁻.

The technology also provides, according to one embodiment, a process for preparing genetically stable therapeutic bacterial strains comprising genetically engineering the therapeutic genes of interest into a bacterially codon optimized expression sequence within a bacterial plasmid expression vector, endogenous virulence (VIR) plasmid (of Salmonella sp.), or chromosomal localization expression vector for any of the deleted genes or IS200 genes, defective phage or intergenic regions within the strain and further containing engineered restriction endonuclease sites such that the bacterially codon optimized expression gene contains subcomponents which are easily and rapidly exchangeable, and the bacterial strains so produced.

The present technology provides, for example, and without limitation, live bacterial compositions that are genetically engineered to express one or more protease inhibitors combined with antigens.

According to various embodiments, the technology provides pharmaceutical compositions comprising pharmaceutically acceptable carriers and one or more bacterial mutants. The technology also provides pharmaceutical compositions comprising pharmaceutically acceptable carriers and one or more bacterial mutants comprising nucleotide sequences encoding one or more peptides. Preferably, the bacterial mutants are attenuated by introducing one or more mutations in one or more genes in the lipopolysaccharide (LPS) biosynthetic pathway (for gram-negative bacteria), and optionally one or more mutations to auxotrophy for one or more nutrients or metabolites.

In one embodiment, a pharmaceutical composition comprises a pharmaceutically acceptable carrier and one or more bacterial mutants, wherein said attenuated bacterial mutants are facultative anaerobes or facultative aerobes. In another embodiment, a pharmaceutical composition comprises a pharmaceutically acceptable carrier and one or more attenuated bacterial mutants, wherein said attenuated bacterial mutants are facultative anaerobes or facultative aerobes. In one embodiment, a pharmaceutical composition comprises a pharmaceutically acceptable carrier and one or more bacterial mutants, wherein said attenuated bacterial mutants are facultative anaerobes or facultative aerobes. In another embodiment, a pharmaceutical composition comprises a pharmaceutically acceptable carrier and one or more attenuated bacterial mutants, wherein said attenuated bacterial mutants are facultative anaerobes or facultative aerobes.

A pharmaceutically effective dosage form may comprise between about 10⁵ to 10¹² live bacteria, within a lyophilized medium for oral administration. In some embodiments, about 10⁹ live bacteria are administered.

Pharmaceutically Acceptable Formulations

Pharmaceutically acceptable formulations may be provided for delivery by other various routes e.g. by intramuscular injection, subcutaneous delivery, by intranasal delivery (e.g. WO 00/47222, U.S. Pat. No. 6,635,246), intradermal delivery (e.g. WO02/074336, WO02/067983, WO02/087494, WO02/0832149 WO04/016281, each of which is expressly incorporated herein by reference it its entirety) by transdermal delivery, by transcutaneous delivery, by topical routes, etc. Injection may involve a needle (including a microneedle), or may be needle-free. See, e.g., U.S. Pat. Nos. 7,452,531, 7,354,592, 6,962,696, 6,923,972, 6,863,894, 6,685,935, 6,475,482, 6,447,784, 6,190,657, 6,080,849 and US Pub. 2003/0059400, each of which is expressly incorporated herein by reference.

Bacterial vector vaccines are known, and similar techniques may be used for the present bacteria as for bacterial vaccine vectors (U.S. Pat. No. 6,500,419, Curtiss, In: New Generation Vaccines: The Molecular Approach, Ed., Marcel Dekker, Inc., New York, N.Y., pages 161-188 and 269-288 (1989); and Mims et al, In: Medical Microbiology, Eds., Mosby-Year Book Europe Ltd., London (1993)). These known vaccines can enter the host, either orally, intranasally or parenterally. Once gaining access to the host, the bacterial vector vaccines express an engineered prokaryotic expression cassette contained therein that encodes a foreign antigen(s). Foreign antigens can be any protein (or part of a protein) or combination thereof from a bacterial, viral, or parasitic pathogen that has vaccine properties (New Generation Vaccines: The Molecular Approach, supra; Vaccines and Immunotherapy, supra; Hilleman, Dev. Biol. Stand., 82:3-20 (1994); Formal et al, Infect. Immun. 34:746-751 (1981); Gonzalez et al, J. Infect. Dis., 169:927-931 (1994); Stevenson et al, FEMS Lett., 28:317-320 (1985); Aggarwal et al, J. Exp. Med., 172:1083-1090 (1990); Hone et al, Microbial. Path., 5:407-418 (1988); Flynn et al, Mol. Microbiol., 4:2111-2118 (1990); Walker et al, Infect. Immun., 60:4260-4268 (1992); Cardenas et al, Vacc., 11:126-135 (1993); Curtiss et al, Dev. Biol. Stand., 82:23-33 (1994); Simonet et al, Infect. Immun., 62:863-867 (1994); Charbit et al, Vacc., 11:1221-1228 (1993); Turner et al, Infect. Immun., 61:5374-5380 (1993); Schodel et al, Infect. Immun., 62:1669-1676 (1994); Schodel et al, J. Immunol., 145:4317-4321 (1990); Stabel et al, Infect. Immun., 59:2941-2947 (1991); Brown, J. Infect. Dis., 155:86-92 (1987); Doggett et al, Infect. Immun., 61:1859-1866 (1993); Brett et al, Immunol., 80:306-312 (1993); Yang et al, J. Immunol., 145:2281-2285 (1990); Gao et al, Infect. Immun., 60:3780-3789 (1992); and Chatfield et al, Bio/Technology, 10:888-892 (1992)). Delivery of the foreign antigen to the host tissue using bacterial vector vaccines results in host immune responses against the foreign antigen, which provide protection against the pathogen from which the foreign antigen originates (Mims, The Pathogenesis of Infectious Disease, Academic Press, London (1987); and New Generation Vaccines: The Molecular Approach, supra). See also: Formal et al, Infect. Immun., 34:746-751 (1981); Wick et al, Infect. Immun., 62:4542-4548 (1994)); Hone et al, Vaccine, 9:810-816 (1991); Tacket et al, Infect. Immun., 60:536-541 (1992); Hone et al, J. Clin. Invest., 90:412-420 (1992); Chatfield et al, Vaccine, 10:8-11 (1992); Tacket et al, Vaccine, 10:443-446 (1992); van Damme et al, Gastroenterol., 103:520-531 (1992) (Yersinia pestis), Noriega et al, Infect. Immun., 62:5168-5172 (1994) (Shigella spp), Levine et al, In: Vibrio cholerae, Molecular to Global Perspectives, Wachsmuth et al, Eds, ASM Press, Washington, D.C., pages 395-414 (1994) (Vibrio cholerae), Lagranderie et al, Vaccine, 11:1283-1290 (1993); Flynn, Cell. Molec. Biol., 40(Suppl.1):31-36 (1994) (Mycobacterium strain BCG), Schafer et al, J. Immunol., 149:53-59 (1992) (Listeria monocytogenes), each of which is expressly incorporated herein by reference.

The bacteria are generally administered along with a pharmaceutically acceptable carrier and/or diluent. The particular pharmaceutically acceptable carrier and/or diluent employed is not critical to the present invention unless otherwise specific herein (or in a respective incorporated referenced relevant to the issue). Examples of diluents include a phosphate buffered saline, buffer for buffering against gastric acid in the stomach, such as citrate buffer (pH 7.0) containing sucrose, bicarbonate buffer (pH 7.0) alone (Levine et al, J. Clin. Invest., 79:888-902 (1987); and Black et al J. Infect. Dis., 155:1260-1265 (1987), expressly incorporated herein by reference), or bicarbonate buffer (pH 7.0) containing ascorbic acid, lactose, and optionally aspartame (Levine et al, Lancet, II:467-470 (1988), expressly incorporated herein by reference). Examples of carriers include proteins, e.g., as found in skim milk, sugars, e.g., sucrose, or polyvinylpyrrolidone. Typically, these carriers would be used at a concentration of about 0.1-30% (w/v) but preferably at a range of 1-10% (w/v).

Set forth below are other pharmaceutically acceptable carriers or diluents which may be used for delivery specific routes. Any such carrier or diluent can be used for administration of the bacteria of the invention, so long as the bacteria are still capable of invading a target cell. In vitro or in vivo tests for invasiveness can be performed to determine appropriate diluents and carriers. The compositions of the invention can be formulated for a variety of types of administration, including systemic and topical or localized administration. Lyophilized forms are also included, so long as the bacteria are invasive upon contact with a target cell or upon administration to the subject. Techniques and formulations generally may be found in Remington's Pharmaceutical Sciences, Meade Publishing Co., Easton, Pa., expressly incorporated herein by reference in its entirety. For systemic administration, injection is preferred, including intramuscular, intravenous, intraperitoneal, and subcutaneous. For injection, the composition, e.g., bacteria, of the invention can be formulated in liquid solutions, preferably in physiologically compatible buffers such as Hank's solution or Ringer's solution.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner.

For administration by inhalation, the pharmaceutical compositions for use according to the present invention are conveniently delivered in the form of an aerosol spray presentation from pressurized packs or a nebulizer, with the use of a suitable propellant, e.g., a hydrofluorocarbon (HFC), carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the composition, e.g., bacteria, and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

See also U.S. Pat. No. 6,962,696, expressly incorporated herein by reference in its entirety.

The present invention provides a pharmaceutical composition comprising a pharmaceutically acceptable carrier and an attenuated tumor-targeted bacteria comprising one or more nucleic acid molecules encoding one or more primary effector molecules operably linked to one or more appropriate promoters. The present invention provides a pharmaceutical composition comprising a pharmaceutically acceptable carrier and an attenuated tumor-targeted bacteria comprising one or more nucleic acid molecules encoding one or more primary effector molecules and one or more secondary effector molecules operably linked to one or more appropriate promoters.

The present invention provides a pharmaceutical composition comprising a pharmaceutically acceptable carrier and a bacterium.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, olive oil, and the like. Saline is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic attenuated tumor-targeted bacteria, in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a suspending agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the pharmaceutical composition of the invention which will be effective in the vaccination of a subject can be determined by standard clinical techniques. In addition, in vitro assays may optionally be employed to help identify optimal dosage ranges. The precise dose to be employed in the formulation will also depend on the route of administration, and should be decided according to the judgment of the practitioner and each patient's circumstances. However, suitable dosage ranges are generally from about 1.0 cfu/kg to about 1×10¹⁰ cfu/kg; optionally from about 1.0 cfu/kg to about 1×10⁸ cfu/kg; optionally from about 1×10² cfu/kg to about 1×10⁸ cfu/kg; optionally from about 1 10⁴ cfu/kg to about 1×10⁸ cfu/kg; and optionally from about 1×10⁴ cfu/kg to about 1×10¹⁰ cfu/kg (cfu=colony forming unit). Effective doses may be extrapolated from dose-response curves derived from in vitro or animal model test systems.

Various delivery systems are known and can be used to administer a pharmaceutical composition of the present invention. Methods of introduction include but are not limited to intradermal, intramuscular, intraperitoneal, intravenous, subcutaneous, intranasal, and oral routes. The compositions may be administered by any convenient route, for example by infusion or bolus injection, by absorption through epithelial or mucocutaneous linings (e.g., oral mucosa, rectal and intestinal-mucosa, etc.) and may be administered together with other biologically active agents. Administration can be systemic or local. Pulmonary administration can also be employed, e.g., by use of an inhaler or nebulizer, and formulation with an aerosolizing agent.

The invention also provides a pharmaceutical pack or kit comprising one or more containers filled with one or more of the ingredients of the pharmaceutical compositions of the invention. Optionally associated with such container(s) can be a notice in the form prescribed by governmental agency regulating the manufacture, use or sale of pharmaceuticals or biological products, which notice reflects approval by the agency of manufacture, use or sale for human administration.

The compositions and methods described herein can be administered to a subject in need of treatment, e.g. in need of treatment for inflammation or cancer. In some embodiments, the methods described herein comprise administering an effective amount of compositions described herein, e.g. engineered microbial cells to a subject in order to alleviate a symptom. As used herein, “alleviating a symptom” is ameliorating any condition or symptom associated with a given condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, subcutaneous, transdermal, airway (aerosol), cutaneous, topical, or injection administration. Administration can be local or systemic.

The term “effective amount” as used herein refers to the amount of engineered microbial cells needed to alleviate at least one or more symptom of the disease or disorder, and relates to a sufficient amount of pharmacological composition to provide the desired effect. The term “therapeutically effective amount” therefore refers to an amount of engineered microbial cells that is sufficient to effect a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the ED50 (the dose therapeutically effective in 50% of the population). The dosage can vary depending upon the dosage form employed and the route of administration utilized. The dose ratio between toxic and therapeutic effects is the therapeutic index and can be expressed as the ratio ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an engineered microbial cell which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for inflammation, among others. The dosage can be determined by a physician and adjusted, as necessary, to suit observed effects of the treatment.

In some embodiments, the technology described herein relates to a pharmaceutical composition comprising an engineered microbial cell as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutical compositions comprising an engineered microbial cell can be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

In certain embodiments, an effective dose of a composition comprising engineered microbial cells as described herein can be administered to a patient once. In certain embodiments, an effective dose of a composition comprising engineered microbial cells can be administered to a patient repeatedly. In some embodiments, the dose can be a daily administration, for example oral administration, of, e.g., a capsule comprising bacterial cells as described herein. In some embodiments, the dose can be, e.g. an injection or gavage of bacterial cells. In some embodiments, the dose can be administered systemically, e.g. by intravenous injection. In some embodiments, a dose can comprise from 10⁶ to 10¹² cells. In some embodiments, a dose can comprise from about 10⁸ to 10¹⁰ cells. A composition comprising engineered microbial cells can be administered over a period of time, such as over a 5 minute, 10 minute, 15 minute, 20 minute, or 25 minute period. The administration can be repeated, for example, on a regular basis, such as every few days, once a week, or biweekly (i.e., every two weeks) for one month, two months, three months, four months or longer.

In some embodiments, after an initial treatment regimen, the treatments can be administered on a less frequent basis. For example, after treatment biweekly for three months, treatment can be repeated once per month, for six months or a year or longer.

The efficacy of engineered microbial cells in, e.g. the raising of an appropriate immune response to a specified disease, e.g., schistosomiasis, can be determined by the skilled clinician. However, a treatment is considered “effective treatment,” as the term is used herein, clinically useful partial or complete immunity is achieved. Efficacy can be assessed, for example, by measuring a marker, indicator, population statistic, or any other measurable parameter appropriate.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, the terms “reduced”, “reduction”, “decrease”, or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or more or any decrease of at least 10% as compared to a reference level. In some embodiments, the terms can represent a 100% decrease, i.e., a non-detectable level as compared to a reference level. In the context of a marker or symptom, a “decrease” is a statistically significant decrease in such level. The decrease can be, for example, at least 10%, at least 20%, at least 30%, at least 40% or more, and is preferably down to a level accepted as within the range of normal for an individual without such disorder. In some instances, the symptom can be essentially eliminated which means that the symptom is reduced, i.e., the individual is in at least temporary remission.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or non-human animal. Usually the non-human animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Animals also include armadillos, hedgehogs, and camels, top name a few. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, cow, or pig, but is not limited to these examples. Mammals other than humans can be advantageously used as subjects that represent animal models of a given condition. A subject can be male or female.

A subject can be one who has been previously diagnosed with or identified as suffering from or having a condition in need of treatment, and optionally, have already undergone treatment. Alternatively, a subject can also be one who has not been previously diagnosed as having a condition. For example, a subject can be one who exhibits one or more risk factors or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operatively linked to appropriate regulatory sequences. A gene may or may not include regions preceding and following the coding region, e.g. 5′ untranslated (5′UTR) or “leader” sequences and 3′ UTR or “trailer” sequences.

The term “operatively linked” includes having an appropriate start signal (e.g., ATG) in front of the polynucleotide sequence to be expressed, and maintaining the correct reading frame to permit expression of the polynucleotide sequence under the control of the expression control sequence, and, optionally, production of the desired polypeptide encoded by the polynucleotide sequence. In some examples, transcription of a nucleic acid is under the control of a promoter sequence (or other transcriptional regulatory sequence) which controls the expression of the nucleic acid in a cell-type in which expression is intended. It will also be understood that the nucleic acid can be under the control of transcriptional regulatory sequences which are the same or which are different from those sequences which control transcription of the naturally-occurring form of a protein.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer or inflammation. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for all purposes, including, but not limited to, describing and disclosing, for example, the methodologies described in such publications that might be used in connection with the technology described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### SUMMARY OF THE INVENTION

Schistosoma mansoni threatens hundreds of millions of people in >50 countries. Schistosomulae (S. mansoni juveniles) migrate through the lung and adult worms reside in blood vessels adjacent to the intestinal mucosa. Current candidate vaccines aren't designed to elicit a mucosal response. We have repurposed an attenuated Salmonella enterica Typhimurium strain (YS1646) to produce such a vaccine targeting Cathepsin B (CatB), a digestive enzyme important for parasite survival. Promoter-Type 3 secretory signal pairs were screened for protein expression in vitro and transfected into YS1646 (also known as VNP 20009) to generate candidate vaccine strains. Two strains were selected for in vivo evaluation (nirB_SspH1 and SspH1_SspH1). Female C57BL/6 mice were immunized twice, 3 weeks apart, using six strategies: i) saline gavage (control), ii) the ‘empty’ YS1646 vector orally (PO) followed by intramuscular (IM) recombinant CatB (20 μg IM rCatB), iii) two doses of IM rCatB, iv) two PO doses of YS1646-CatB, v) IM rCatB then PO YS1646-CatB and vi) PO YS1646-CatB then IM rCatB. Serum IgG responses to CatB were monitored by ELISA. Three weeks after the second dose, mice were challenged with 150 cercariae and sacrificed 7 weeks later to assess adult worm and egg burden (liver and intestine), granuloma size and egg morphology. CatB-specific IgG antibodies were low/absent in the control and PO only groups but rose substantially in other groups (5898-6766 ng/mL). The highest response was in animals that received nirB_SspH1 YS1646 PO then IM rCatB. In this group, reductions in worm and intestine/liver egg burden (vs. control) were 93.1% and 79.5%/90.3% respectively (all P<0.0001). Granuloma size was reduced in all vaccinated groups (range 32.86-52.83×10³ μm²) and most significantly in the nirB_SspH1+CatB IM group (34.74±3.35×10³ μm² vs. 62.22±6.08×10³ μm²: vs. control P<0.01). Many eggs in the vaccinated animals had abnormal morphology. Targeting CatB using a multi-modality approach can provide almost complete protection against S. mansoni challenge.

The complete catB gene was successfully transfected into YS1646 using a number of promoters and secretory signals. Plasmid-bearing YS1646 strains were screened in vitro and several were advanced to the mouse model where they were immunogenic when administered in two doses 3 weeks apart (IM twice, PO twice, PO then IM, IM then PO).

The protection elicited by several of these schedules was among the highest ever reported for a Schistosome vaccine in mice: 80-93% reductions in worms and liver/intestinal egg burden. YS1646-based vaccination was also immunogenic and protective in the shorter IM+PO schedule described above (1×1 dose+3× PO doses over 5 days). Egg granulomas were much smaller in vaccinated animals and egg morphology was grossly abnormal suggesting decreased viability. The short schedule was also surprisingly effective at treating established S. mansoni infection in mice. When mice were vaccinated 1 month after infection and followed for 2 months, there were major reductions in worm burden (63.2±5%) and both intestinal (62.7±7%) and liver egg burden (58.2±4%).

Theoretical Advantages of a YS1646-Based S. mansoni vaccine include immunity in the gut and the systemic circulation for a parasite that ‘wanders’, induction of both humoral (antibodies) and cellular (T cell) immunity for durability, the same vaccine antigen might have both prophylactic and therapeutic uses, use of either adjuvanted or non-adjuvanted dosage forms, convenience of dosing requiring only 1 visit for i.m. injection, with boosting using p.o. dosage form, potential for lyophilization of bacteria using simple sugars with a buffer for p.o. dosing, and targeting of the vaccine against CatB appears to have both therapeutic and preventative activity.

While experiments to date have employed plasmid loci for the gene encoding the antigenic peptide, chromosomal integration of ‘one-copy’ YS1646 vaccines and ‘multi-copy’ YS1646 vaccines are also provided herein. YS1646 is highly attenuated in humans, and one strategy to reduce the attenuation is to employ zwf deficient bacteria, which relieves sensitivity of the bacteria to low pH, high CO₂, and high osmolarity.

One embodiment of the technology provides an S. mansoni preventative vaccine or kit therefor, for example, a YS1646 vaccine bearing a single copy or multiple copies of CatB that would be used in either a PO only schedule (ie: 3 doses every other day) or a multi-modality schedule (ie: 1 dose IM with 3 doses PO over 5 days). While the experiments reported herein and that of Chen with their YS1646 vaccine targeting S. japonicum suggest that the multimodality schedule will be superior, in some cases an oral-only vaccine may be preferred. Other Schistosome antigens may also be included, or provided as an alternate to CatB. The IM dose may be adjuvanted or unadjuvanted.

Another embodiment provides an S. mansoni therapeutic vaccine. Because S. mansoni modifies the human immune response to ensure its own survival, a therapeutic vaccine might need to provide a stronger ‘push’ to the immune system to have therapeutic impact. Therefore, a therapeutic regimen preferably includes at least one adjuvanted IM dose, in addition to the oral YS1646 doses.

Salmonella type-III secretion systems (T3SS) and both T3SS-specific and constitutive promoters were exploited, to generate a panel of YS1646 strains with plasmid-based expression of enhanced green fluorescent protein (eGFP) or full-length CatB. This panel was screened for protein expression in monomicrobial culture and murine RAW 264.7 murine macrophages and the most promising constructs were advanced to in vivo testing in adult female C57BL/6 mice. Animals were vaccinated with the two most promising strains using several strategies and then subjected to cercarial challenge.

A two-dose, multimodality schedule starting with oral (PO) gavage of YS1646 bearing the nirB_SspH1_CatB plasmid followed by intramuscular (IM) recombinant CatB (rCatB) was able to reduce both worm and tissue egg burden by 80-90%. Such reductions are among the best ever reported for any S. mansoni candidate vaccine in a murine model.

Promoter-Type 3 secretory signal pairs were screened for protein expression in vitro and transfected into YS1646 to generate candidate vaccine strains. Two strains were selected for in vivo evaluation (nirB_SspH1 and SspH1_SspH1). Female C57BL/6 mice were immunized twice, 3 weeks apart, using six strategies: i) saline gavage (control), ii) the ‘empty’ YS1646 vector orally (PO) followed by intramuscular recombinant CatB (20 μg IM rCatB), iii) two doses of IM rCatB, iv) two PO doses of YS1646-CatB, v) IM rCat then PO YS1646-CatB and vi) PO YS1646-CatB then IM rCatB. Serum IgG responses to CatB were monitored by ELISA. Three weeks after the second dose, mice were challenged with 150 cercariae and sacrificed 7 weeks later to assess adult worm and egg burden (liver and intestine), granuloma size and egg morphology.

CatB-specific antibodies were low/absent in the control and PO only groups but rose substantially in other groups (5898-6766 ng/mL). The highest response was in animals that received nirB-SspH1 YS1646 PO then IM rCat. In this group, reductions in worm and intestine/liver egg burden (vs. control) were 93.1% and 79.5%/90.3% respectively (all P<0.0001). Granuloma size was reduced in all vaccinated groups (range 32.86-52.83×10³ μm²) and most significantly in the nirB_SspH1+CatB IM group (34.74±3.35 μm² vs. 62.22±6.08×10³ μm²: vs. control P<0.01). Many eggs in the vaccinated animals had abnormal morphology.

Targeting CatB using a multi-modality approach can provide almost complete protection against S. mansoni challenge.

Salmonella type-III secretory signals (T3SS) and both T3SS-specific and constitutive promoters were exploited to generate a panel of YS1646 strains with plasmid-based expression of enhanced green fluorescent protein (eGFP) or full-length CatB that were screened for protein expression in monomicrobial culture and murine RAW 264 murine macrophages. Promising constructs were advanced to in vivo testing in adult female C57BL/6 mice Animals were vaccinated with the two most promising strains using several strategies and then subjected to cercarial challenge. A two-dose, multimodality schedule starting with oral (PO) gavage of YS1646 bearing the nirB-SspH1-CatB plasmid followed by intramuscular (IM) recombinant CatB (rCatB) was able to reduce both worm and tissue egg burden by 80-90%. Such reductions are among the best ever reported for any S. mansoni candidate vaccine.

CatB-specific IgG levels were low or absent in the saline control and PO only groups but rose substantially in all other vaccine groups (5898-6766 ng/mL). Responses were highest (6766±2128 ng/mL) in the animals immunized with the nirB-SspH1 YS1646 followed by IM catB. All vaccinated animals had reduced worm (70-89%) and egg burden in both the intestines (57-78%) and liver (67-88%). In the nirB-SspH1 plus catB IM group, the worm and intestine/liver egg burden were reduced 89% and 78.7%/87.9% (all P<0.0001). Overall granuloma size was significantly reduced in all vaccinated groups: again, most significantly in the nirB-SspH1 plus catB IM group. Many of the eggs in the granulomas of vaccinated animals had abnormal morphology.

Targeting the digestive enzyme catB using a multimodality approach (i.e., PO using a S. enterica Typhimurium vector plus a single IM dose) elicits both systemic and mucosal immune responses and provides almost complete protection against S. mansoni challenge.

These YS1646-vectored candidate vaccines show considerable promise to address the currently unmet need for an S. mansoni vaccine.

It is therefore an object to provide pharmaceutically acceptable orally-administrable vaccine formulation, comprising: an attenuated recombinant Salmonella bacterium adapted for colonization of a human gut, expressing at least one antigen corresponding to at least one Schistosome antigen; and a pharmaceutically acceptable carrier adapted to preserve the attenuated Salmonella bacterium through the gastrointestinal tract for delivery in the human gut.

The at least one antigen may be secreted from the Salmonella bacteria by a Salmonella Type 3 secretion system.

The at least one antigen may be selected from the group consisting of CatB.

The at least one antigen may be expressed in a fusion peptide with a secretory signal selected from the group consisting of one or more of SopE2, SseJ, SptP, SspH1, SspH2, SteA, and SteB.

The transcription of the at least one antigen may be under control of at least one promoter selected from the group consisting of one or more of SopE2, SseJ, SptP, SspH1, SspH2, SteA, SteB, pagC, lac, nirB, and pagC.

The at least one antigen may be produced based on a chromosomally integrated genetically engineered construct and/or a plasmid genetically engineered construct.

The at least one antigen may be produced based on a genetically engineered construct comprising a promoter portion, a secretion signal portion, and an antigen portion.

The promoter portion and the secretion signal portion may be separated by a first restriction endonuclease cleavage site. The secretion signal portion and the antigen portion may also be separated by a second restriction endonuclease cleavage site.

The genetically engineered construct may comprise plasmid, further comprising an antibiotic resistance gene.

It is another object to provide a recombinant attenuated Salmonella bacterium adapted for growth in a mammal, expressing at least one antigen corresponding to Schistosome antigen, adapted to induce an vaccine response to schistosome after oral administration to the mammal.

It is a further object to provide a method of immunizing a human against infection by schistosomes, comprising orally administering a pharmaceutically acceptable formulation, comprising: an attenuated recombinant Salmonella bacterium, expressing at least one antigen corresponding to a schistosome antigen; and a pharmaceutically acceptable carrier adapted to preserve the attenuated Salmonella bacterium through the gastrointestinal tract for delivery in the human gut.

The method may further comprise administering a second pharmaceutically acceptable formulation comprising at least one antigen corresponding to at least one schistosome antigen, through a non-oral route of administration.

The non-oral route of administration may comprise an intramuscular route of administration. The second pharmaceutically acceptable formulation may comprise an adjuvant.

The administration of the first pharmaceutically acceptable formulation and second pharmaceutically acceptable formulation may be concurrent, or the first pharmaceutically acceptable formulation may precede or succeeds the administering of the second pharmaceutically acceptable formulation. The administering of the first and/or second pharmaceutically acceptable formulation may be dependent on a test of pre-existing immunity of the human.

The administering of the first pharmaceutically acceptable formulation and the second pharmaceutically acceptable formulation may be according to a prime-pull, prime-boost or alternate administration protocol.

The administering of the first pharmaceutically acceptable formulation and the second pharmaceutically acceptable formulation may be in a manner dependent on tests of at least IgG and IgA immune response.

The administering of the first pharmaceutically acceptable formulation and the second pharmaceutically acceptable formulation are preferably effective to produce both IgG and IgA immunity to schistosomes.

These results represent between X-Y animals/group from Z experiments. (**P<0.01, ***P<0.005, ****P<0.0001)

Each plasmid construct was cloned to express S. mansoni-Cathepsin B (Sm-CatB) or enhanced green fluorescent protein (eGFP) fused with a type-3 secretory signal from S. enterica Typhimurium and driven by promoters from E. coli or S. enterica Typhimurium. Plasmid nomenclature=‘Promoter_T3SS_Gene of Interest’.

It is another object to provide a vaccine adapted to raise immunity to Shistosomes in animals, comprising an attenuated recombinant bacterium adapted to secrete CatB.

The attenuated recombinant bacterium may be YS1646 or YS1646 zwf−.

The vaccine may be provided in a kit with an i.m. dosage form of CatB or adjuvanted CatB.

It is a further object to provide a method of immunizing an animal against a parasitic worm, comprising enterically administering at least one dose of a live attenuated recombinant bacterium genetically engineered to secrete a parasitic worm digestive enzyme antigen in the animal's gut.

The attenuated recombinant bacterium may comprise YS1646 or YS1646 zwf−.

The parasitic worm may comprise a schistosome, e.g., S. mansoni. The parasitic worm digestive enzyme antigen may comprise catB.

The method may further comprise parenterally administering at least one dose of a purified antigen parasitic worm digestive enzyme antigen to the animal, e.g., prior to enterically administering the live attenuated recombinant bacterium.

The at least one dose of a purified antigen parasitic worm digestive enzyme antigen may be provided in a dosage form comprising an adjuvant.

The animal may be uninfected with the parasitic worm, and the animal may develop a preventative immune response to the parasitic worm. The animal may be infected with the parasitic worm, and the animal may develop a therapeutic immune response to the parasitic worm.

The enteric administration of at least one dose of a live attenuated recombinant bacterium genetically engineered to secrete the parasitic worm digestive enzyme antigen in the animal's gut may be repeated at least once, e.g., at least twice, with at least 24 hours between doses. The enteric administration may be preceded by at least one parenteral dose of the parasitic worm digestive enzyme antigen, and the enterically administering may be thereafter repeated at least once with at least 24 hours between enteric doses.

### BRIEF DESCRIPTION OF THE DRAWINGS

FIG. 1 shows a plasmid map for a recombinant plasmid. The pQE-30 plasmid served as a backbone. The promoter and secretory signal were inserted between the Xho1 and Not1 restriction sites. The full-length Cathepsin B gene, was inserted between the Not1 and Asc1 sites. An ampicillin resistance gene was used as a selectable marker.

FIG. 2 shows an immunization schedule. Baseline serum was collected on day 0 for all mice. Depending on the experimental group, mice receive 3 oral doses of YS1646 (1×10⁹ cfu/dose) or PBS every other day while others receive an intramuscular dose of 20 μg of CatB on day 5. Mice were bled and underwent a second round of vaccination three weeks later before being challenged with 150 S. mansoni cercariae by tail penetration. All animals were sacrificed 6-7 weeks post-infection.

FIGS. 3A-3C show expression of recombinant cathepsin B. FIG. 3A shows the plasmids nirB_SspH1, SspH1_SspH1 and SteA_SteA were transformed into Salmonella strain YS1646. Whole bacteria lysates and monomicrobial culture supernatants were examined for the presence of CatB by western blot. FIG. 3B shows the mouse macrophage cell line RAW 264.7 cells were infected with transformed YS1646 strains expressing eGFP as a marker for the capacity of promoter-TSSS pairs to support expression of a foreign protein. DAPI nuclear stain is represented in blue and eGFP is shown in green. Scale at 100 μm. FIG. 3C shows mouse macrophage cells line RAW 264.7 cells were infected with selected plasmids from Table 1 and the presence of CatB protein was determined by western blotting.

FIGS. 4A-4E show production of Sm-Cathepsin B specific antibodies prior to challenge. Serum anti-CatB IgG was measured by ELISA at weeks 0, 3 and 6 for groups that received the nirB_SspH1 construct (FIG. 4A) or the SspH1_SspH1 construct (FIG. 4B). These results represent between 8-16 animals/group from 2 independent experiments. FIG. 4C shows serum anti-CatB IgG1 and IgG2c were measured by endpoint-dilution ELISA and expressed as the ratio of IgG1/IgG2c. FIG. 4D shows intestinal anti-CatB IgA in intestinal tissue was measured by ELISA and is reported as mean±standard error of the mean ng/gram. These results represent 5-7 animals per group. (*P<0.05, **P<0.01, ***P<0.001 compared to the PBS group). FIG. 4E shows intestinal anti-CatB IgG measured by ELISA and is reported as mean±standard error of the mean ng/gram. Statistical test: One-way ANOVA, Tukeys multiple comparison (P<0.001).

FIGS. 5A-5B show cytokine production prior to challenge. Supernatant IL-5 (FIG. 5A) and IFN-γ (FIG. 5B) levels after stimulating splenocytes with rCatB for 72 hours were measured by QUANSYS multiplex ELISA. These results represent 5-7 animals per group. Results are expressed as the mean+the standard error of the mean. (*P<0.05, **P<0.01 compared to the PBS group)

FIGS. 6A-6C show parasitologic burden. The reduction in worm counts (FIG. 6A) as well as the reduction in egg load per gram of liver (FIG. 6B) or intestine (FIG. 6C) are represented for mice in the PBS, empty vector, PO, IM, and multimodality groups. Worm and egg burdens were determined 7 weeks after cercarial challenge. These results represent between 8-16 animals/group from 2 independent experiments. (*P<0.05, **P<0.01, ***P<0.001, ****P<0.0001 compared to the PBS group).

FIGS. 7A and 7B show histological staining of liver granulomas, including representative images of H&E staining of granulomas from livers of vaccinated mice (FIG. 7A, PO→IM group for the nirB_SspH1 construct) and saline control mice (FIG. 7B). Scale is set to 100 μm.

FIGS. 8A-8C show reductions in adult worms (FIG. 8A), eggs in liver (FIG. 8B), and eggs in intestines (FIG. 8C), two months after infection and four weeks after vaccination (two replicate experiments with ˜12 animals in each group).

FIGS. 9A-9C show reductions in adult worms (FIG. 9A), eggs in liver (FIG. 9B), and eggs in intestines (FIG. 9C), two months after infection and eight weeks after vaccination (two replicate experiments with ˜12 animals in each group).

FIGS. 10A-10C show reductions in adult worms (FIG. 10A), eggs in liver (FIG. 10B), and eggs in intestines (FIG. 10C), four months after infection and eight weeks after vaccination (one experiment with ˜8 animals in each group).

FIGS. 11A-11C show reductions in adult worms (FIG. 11A), eggs per gram in liver (FIG. 11B), eggs in intestine (FIG. 11C), over six months after infection.

### DETAILED DESCRIPTION OF THE PREFERRED EMBODIMENTS

Methods

All animal procedures were conducted in accordance with Institutional Animal Care and Use Guidelines and were approved by the Animal Care and Use Committee at McGill University (Animal Use Protocol 7625).

Plasmids

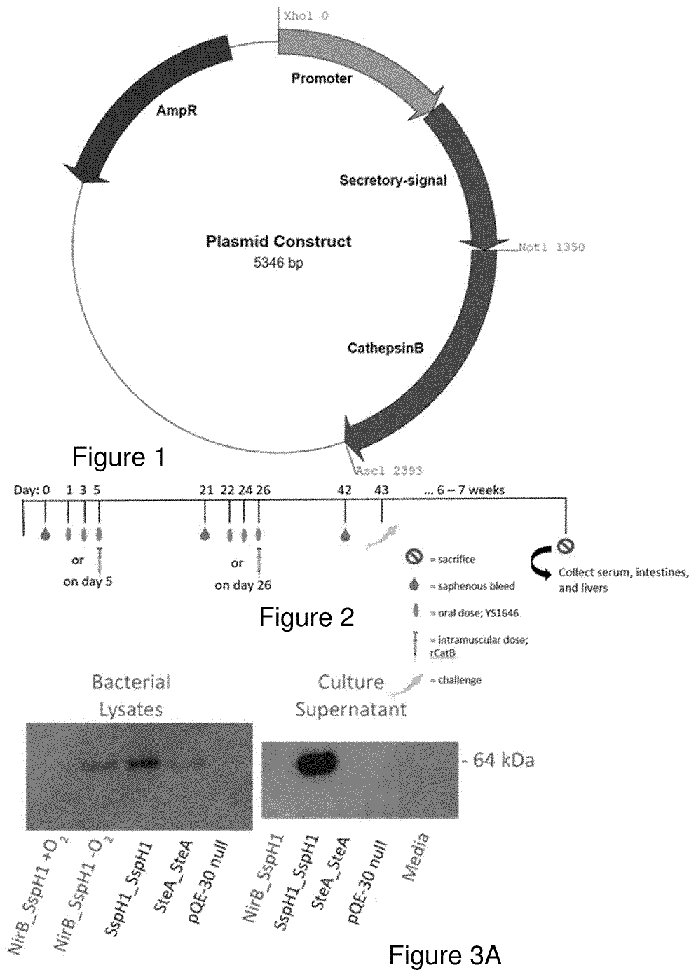

Gene segments of the pagc promoter as well as the sopE2, sspH1, sspH2, sptP, steA, steB and steJ promoters and secretory signals were cloned from YS1646 genomic DNA (American Type Culture Collection, Manassas, Va.) and the nirB and lac promoters were cloned from E. coli genomic DNA (strain AR_0137) (ThermoFischer Scientific, Eugene, Oreg.). S. mansoni CatB complementary DNA (cDNA) was sequence-optimized for expression in S. enterica Typhimurium [Java Codon Optimization Tool (jcat)], synthesized by GenScript (Piscataway, N.J.) and inserted into the pUC57 plasmid with a 6× His tag at the 3′ end. Promoter-T3SS pairs were cloned upstream of the CatB gene and inserted separately into pQE30 (Qiagen, Hilden, Germany). Parallel constructs were made with CatB gene replaced by eGFP to produce expression plasmids used for imaging studies. See FIG. 1 for the general plasmid map and Table 1 for a summary of the expression cassettes produced. All plasmids were sequenced to verify successful cloning (McGill Genome Centre, Montreal, QC). S. enterica Typhimurium YS1646 (Cedarlane Labs, Burlington, ON) was cultured in Lysogeny broth (LB) media and strains bearing each construct were generated by electroporation (5 ms, 3 kV: Biorad, Hercules, Calif.). Successfully transformed strains were identified using LB agar containing 50 μg/mL ampicillin (Wisent Bioproducts, St-Bruno, QC). Aliquots of each transformed strain were stored in LB with 15% glycerol at −80° C. until used in experiments.

FIG. 1 shows a plasmid map for recombinant YS1646 strains. The pQE-30 plasmid served as a backbone. The promoter and secretory signal were inserted between the Xho1 and Not1 restriction sites. The full-length Cathepsin B gene was inserted between the Not1 and Asc1 sites. An ampicillin resistance gene was used as a selectable marker.

TABLE 1
Recombinant Salmonella constructs.

| Plasmid | Promoter | Secretory Signal | Protein | |
|---|---|---|---|---|
| Lac_SopE2 | Lac | SopE2 | Sm-Cathepsin B | eGFP |
| nirB_SopE2 | nirB | | | |
| pagC_SopE2 | pagC | | | |
| SopE2_SopE2 | SopE2 | | | |
| Lac_SspH1 | Lac | SspH1 | | |
| nirB_SspH1 | nirB | | | |
| pagC_SspH1 | pagC | | | |
| SspH1_SspH1 | SspH1 | | | |

| | |
|---|---|
| SspH2_SspH2 | SspH2 |
| SteA_SteA | SteA |
| SteB_SteB | SteB |
| SteJ_SteJ | SteJ |
| SptP_SptP | SptP |

Table 1 shows Recombinant Salmonella constructs. Each plasmid construct was cloned to express S. mansoni-Cathepsin B (Sm-CatB) or enhanced green fluorescent protein (eGFP) fused with a type-3 secretory signal from S. enterica Typhimurium and driven by promoters from E. coli or S. enterica Typhimurium. Construct nomenclature=‘Promoter_Secretory Signal_Protein of Interest’.

Table 2 shows primers used in the construct design.

TABLE 2
Primers Used in the Construct Design

| | Forward Primer (5′ → 3′) | Reverse Primer (3′ → 5′) | Source |
|---|---|---|---|
| SopE2 promoter | CCGCTCGAGTAAAAATGT | CATGGTAGTTCTCCTTTTAG | YS1646 |
| and secretory | TCCTCGATAAA | SEQ ID NO: 002 | |
| signal | SEQ ID NO: 001 | | |
| SptP promoter | CGCCTCGAGTTTACGCTG | CATTTTTCTCTCCTCATA | YS1646 |
| and secretory | ACTCATTGG | CTTTA | |
| signal | SEQ ID NO: 003 | SEQ ID NO: 004 | |
| SseJ promoter | CGCCTCGAGACATAAAAC | CGCCTCGAGACATAAAAC | YS1646 |
| and secretory | ACTAGCACT | ACTAGCACT | |
| signal | SEQ ID NO: 005 | SEQ ID NO: 006 | |
| SspH1 promoter | CGCCTCGAGCGCTATATC | CTCTGCGGCCGCGGTAAG | YS1646 |
| and secretory | ACCAAAAC | ACCTGACGCTC | |
| signal | SEQ ID NO: 007 | SEQ ID NO: 008 | |
| SspH2 promoter | CGCCTCGAGGTTTGTGCG | CTCTGCGGCCGCATTCAG | YS1646 |
| and secretory | TCGTAT | GCAGGCACGCA | |
| signal | SEQ ID NO: 009 | SEQ ID NO: 010 | |
| SteA promoter | CGCCTCGAGGTTTCGCCG | CTCTGCGGCCGCATAATT | YS1646 |
| and secretory | CATGTTG | GTCCAAATAGT | |
| signal | SEQ ID NO: 011 | SEQ ID NO: 012 | |
| SteB promoter | CGCCTCGAGCGCTCCAGC | CTCTGCGGCCGCTCTGAC | YS1646 |
| and secretory | GCTTCGA | ATTACCATTT | |
| signal | SEQ ID NO: 013 | SEQ ID NO: 014 | |
| Lac promoter | CGCCTCGAGCATTAGGCACCC | GTGGAATTGTGAGCGGAT | Sequence |
| | CAGGCTTTACACTTTATGCTT | AACAATTTCACACAGGAA | is in the |
| | CCGGCTCGTATGTTGTGTGGA | ACAGCTATGACCATGACT | primers |
| | ATTGTGAGCGGATAA | AACATAACA CTATCCAC | |
| | SEQ ID NO: 015 | SEQ ID NO: 016 | |
| nirB promoter | CGCCTCGAGTTGTGGTTA | CGCGCGGCCGCCGGATCT | DHS a E. |
| | CCGGCCCGAT | TTACTCGCATTAC | coli |
| | SEQ ID NO: 017 | SEQ ID NO: 018 | |
| pagC promoter | CGCCTCGAGGTTAACCAC | AACAACTCCTTAATACTACT | YS1646 |
| | TCTTAATAA | SEQ ID NO: 020 | |
| | SEQ ID NO: 019 | | |
| SopE2 | GGCGGTAATAGAAAAGAA | AAGTCGCGGCCGCCGGAT | YS1646 |
| Secretion | ATCGAGGCAAAAATGACT | CTTTACTCGC | |
| Signal | AACATAACACTATCCAC | SEQ ID NO: 022 | |
| | SEQ ID NO: 021 | | |
| SspH1 | GGCGGTAATAGAAAAGAA | CTCTGCGGCCGCGGTAAG | YS1646 |
| Secretion | ATCGAGGCAAAAATGTTTA | ACCTGACGCTC | |
| Signal | ATATCCGCAATACACAACCTT | SEQ ID NO: 024 | |
| | SEQ ID NO: 023 | | |
| Cathepsin B | CGCGCGGCCGCGCACATC | AGTCGGCGCGCCGTGGTG | S. mansoni |
| | TCTGTTAAAAACGAA | GTGGTGGTGGTGCGG | |
| | SEQ ID NO: 025 | SEQ ID NO: 025 | |
| eGFP | CGCGCGGCCGCGGTGAGC | AGTCGGCGCGCCTTACTT | pEGFP_C1 |
| | AAGGGCGAG | GTACAGCTCGTC | |
| | SEQ ID NO: 027 | SEQ ID NO: 028 | |

Western Blotting

Recombinant YS1646 strains were grown in LB broth with 50 μg/mL ampicillin at 37° C. in a shaking incubator under aerobic or low oxygen (sealed twist-cap tubes) conditions. Bacterial lysates were prepared by centrifugation (9,000×g for 5 min) then boiling the pellet (100° C.×10 min). Proteins from the culture supernatant were precipitated with 10% trichloroacetic acid for 1 hour on ice followed by centrifugation (9,000×g for 2 min) and removal of the supernatant. Protein pellets were resuspended in NuPAGE LDS sample buffer and NuPAGE reducing agent according to the manufacturer's instructions (Thermo Fisher). Immunoblotting was performed as previously described [12]. Briefly, samples were run on a 4-12% Bis-Tris PAGE gel and transferred to nitrocellulose membranes (Thermo Fisher). Membranes were incubated in blocking buffer (5% skim milk in PBS [pH 7.4; 0.01M phosphate buffer, 0.14 M NaCl]) for 1 hour at room temperature (RT) with gentle agitation then washed three times in wash buffer (PBS [pH 7.4; 0.01M phosphate buffer, 0.14 M NaCl], 0.1% Tween 20 (Sigma-Aldrich, St. Louis, Mo.). Membranes were incubated with a murine, monoclonal anti-polyhistidine primary antibody (1:2,500; Sigma-Aldrich) in blocking buffer overnight at 4° C. with gentle shaking. Membranes were washed three times in wash buffer then incubated with a goat, anti-mouse IgG-horseradish peroxidase secondary antibody (1:5000; Sigma-Aldrich) in blocking buffer for 1 hour at RT with gentle agitation. Membranes were washed three times followed by addition of Supersignal West Pico chemiluminescent substrate (Thermo Fisher) as per the manufacturer's instructions and developed using an autoradiography cassette and the X-OMAT 2000 processor system (Kodak, Rochester, N.Y.).

In Vitro Macrophage Infection

Murine macrophage-like cells (RAW 264.7: ATCC-TIB 71) were seeded at 10⁶ cells/well in 12-well plates in Dulbecco's Modified Eagle's medium (DMEM) (Wisent Bioproducts) supplemented with 10% fetal bovine serum (FBS: Wisent Bioproducts). Transformed YS1646 were diluted in DMEM-FBS to give a multiplicity of infection of 100 and centrifuged onto the monolayer (110×g for 10 min) to synchronize the infection. After 1 hour at 37° C. in 5% CO₂, plates were washed three times with phosphate buffered saline (PBS: Wisent Bioproducts) and replaced in the incubator with DMEM-FBS containing 50 μg/mL gentamicin (Sigma-Aldrich) to kill any extracellular bacteria and prevent re-infection. After 2 hours, the cells were washed with PBS three times and the gentamicin concentration was lowered to 5 μg/mL. After 24 hours, the cells were harvested, transferred to Eppendorf tubes and centrifuged (400×g for 5 min). Pellets were prepared for western blotting as above. For imaging experiments, RAW 264.7 cells were seeded into 6-well chamber slides at 10⁴ cells/well and cultured as above. After 24 hours, the cells were stained with 4′,6-diamidino-2-phenylindole (DAPI) (Thermo Fisher), fixed with 4% paraformaldehyde in PBS and incubated for 10 min at RT. Images were obtained using a Zeiss LSM780 laser scanning confocal microscope and analyzed using ZEN software (Zeiss, Oberkochen, Germany).

Purification of Recombinant Cathepsin B

S. mansoni CatB was cloned and expressed in Pichia pastoris as previously described [12]. Briefly, the yeast cells were cultured at 28° C. with shaking in buffered complex glycerol medium (BMGY) (Fisher Scientific, Ottawa, ON). After two days, cells were pelleted (3,000×g for 5 min) and resuspended in fresh BMMY to induce protein expression. After 3 further days of culture, cells were harvested (3,000×g for 5 min) and supernatants were collected and purified by Ni-NTA affinity chromatography. Immunoblotting for the His-tag (as above) confirmed successful expression of CatB. Protein concentration was estimated by Piece bicinchoninic acid assay (BCA) (Thermo Fisher) and aliquots of the rCatB were stored at −80° C. until used.

Immunization Protocol

Female 6-8 week old C57BL/6 mice were purchased from Charles River Laboratories (Senneville, QC). All animals received two doses three weeks apart (See FIG. 2 for experimental design). Oral dosing (PO) was accomplished by gavage three times every other day (200 μL containing 1×10⁹ colony-forming units (CFUs)/dose). Intramuscular (IM) vaccinations were administered using a 25 g needle in the lateral thigh (20 μg rCatB in 50 μL PBS). Each experiment included six groups with 8 mice/group: i) saline PO twice (Control or PBS) ii) YS1646 transformed with ‘empty’ pQE30 vector (EV) PO followed by rCatB IM (EV→IM), iii) CatB-bearing YS1646 PO twice (PO→PO), iv) rCatB IM twice (IM→IM), v) CatB-bearing YS1646 PO followed by rCatB IM (PO→IM), and vi) rCatB IM followed by CatB-bearing YS1646 PO (IM→PO).

FIG. 2 shows an immunization schedule. Baseline serum was collected on day 0 for all mice. Each group consists of either a saline control, EV→IM, PO→PO, IM→IM, PO→IM, and IM→PO for the nirB_SspH1 and/or the SspH1_SspH1 construct. Mice receive 3 oral doses of YS1646 (1×10⁹cfu/dose) or PBS every other day while others receive an intramuscular dose of 20 μg of CatB on day 5. Mice were bled and underwent a second round of vaccination three weeks later before being challenged with 150 S. mansoni cercariae by tail penetration. All animals were sacrificed 6-7 weeks post-infection.

Intestine Processing for IgA Assessment

Four weeks after the second vaccination, the animals were sacrificed, and 10 cm of the proximal small intestine was collected. Tissue was weighed and stored in a protease inhibitor cocktail (Sigma Aldrich) at a 1:5 dilution (w/v) on ice until processed. Tissue was homogenized (Homogenizer 150; Fisher Scientific), centrifuged at 2500×g at 4° C. for 30 minutes and the supernatant was collected. Supernatants were stored at −80° C. until analyzed by ELISA.

Humoral Response by Enzyme-Linked Immunosorbent Assay (ELISA)

Serum IgG and Intestinal IgA

Blood was collected from the saphenous vein at baseline (week 0) and at 3 and 6 weeks in microtainer serum separator tubes (BD Biosciences, Mississauga, ON, Canada). Cleared serum samples were obtained following the manufacturer's protocol and stored at −20° C. until used. Serum CatB-specific IgG and intestinal CatB-specific IgA levels were assessed by ELISA as previously described [30]. Briefly, U-bottom, high-binding 96-well plates (Greiner Bio-One, Frickenhausen, Germany) were coated overnight at 4° C. with rCatB (0.5 μg/mL) in 100 mM bicarbonate/carbonate buffer at pH 9.6 (50 μL/well). Each plate contained a standard curve with 2-fold dilutions of purified mouse IgG or IgA (Sigma Aldrich, St. Louis, Mo.) starting at 2,000 ng/mL. The plates were washed three times with PBS (pH 7.4) and incubated with blocking buffer (2% bovine serum albumin (Sigma-Aldrich) in PBS-Tween 20 (0.05%; Fisher Scientific)) at 37° C. for 1 hour. The plates were washed three times with PBS and diluted serum samples (1:50 in blocking buffer) were added in duplicate (50 μL/well). Blocking buffer was added to the standard curve wells. After 1 hour at 37° C., the plates were washed with PBS four times and horseradish peroxidase-conjugated anti-mouse IgG or horseradish peroxidase-conjugated anti-mouse IgA (Sigma Aldrich) diluted 1:20,000 (1:10,000 for IgA) in blocking buffer was added for 30 min (IgG) or 1 hour (IgA) at 37° C. (75 μL/well). Plates were washed with PBS six times and 3,3′,5,5′-Tetramethyl benzidine (TMB) substrate (100 μL/well; Millipore, Billerica, Mass.) was used for detection followed by 0.5 M H₂SO₄ after 15 min (50 μl/well; Fisher Scientific). Optical density (OD) was measured at 450 nm with an EL800 microplate reader (BioTek Instruments Inc., Winooski, Vt.). The concentration of CatB-specific IgG and IgA were calculated by extrapolation from the mouse IgG or IgA standard curves.

Serum IgG1 and IgG2c

Serum CatB-specific IgG1 and IgG2c levels were assessed by ELISA as previously described [12]. Briefly, Immulon 2HB flat-bottom 96-well plates (Thermo Fisher) were coated overnight at 4° C. with rCatB (0.5 μg/mL) in 100 mM bicarbonate/carbonate buffer at pH 9.6 (50 μL/well). The plates were washed three times with PBS-Tween 20 (PBS-T: 0.05%; Fisher Scientific) and were blocked as above for 90 min. Serial serum dilutions in duplicate were incubated in the plates for 2 hours. Control (blank) wells were loaded with PBS-T. After washing three times with PBS-T, goat anti-mouse IgG1-horseradish peroxidase (HRP) (Southern Biotechnologies Associates, Birmingham, Ala.) and goat anti-mouse IgG2c-HRP (Southern Biotechnologies Associates) were added to the plates and incubated for 1 hour at 37° C. After a final washing step, TMB substrate (50 μL/well; Millipore, Billerica, Mass.) was used for detection followed by 0.5 M H₂SO₄ after 15 min (25 μl/well; Fisher Scientific). Optical density (OD) was measured at 450 nm with an EL800 microplate reader (BioTek Instruments Inc.). The results are expressed as the mean IgG1/IgG2c ratio of the endpoint titers ±standard error of the mean. Endpoint titers refer to the reciprocal of the highest dilution that gives a reading above the cut-off calculated as previously described [31].

Cytokine Production by Multiplex ELISA

In some experiments, some of the animals were sacrificed 4 weeks after the second vaccination. Spleens were collected and splenocytes were isolated as previously described with the following modifications [13]. Splenocytes were resuspended in 96-well plates (10⁶ cells/well) in RPMI-1640 (Wisent Bioproducts) supplemented with 10% fetal bovine serum, 1 mM penicillin/streptomycin, 10 mM HEPES, 1× MEM non-essential amino acids, 1 mM sodium pyruvate, 1 mM L-glutamine (all from Wisent Bioproducts), 0.05 mM 2-mercaptoethanol (Sigma-Aldrich). The cells were incubated at 37° C. in the presence of 2.5 μg/mL of rCatB for 72 hours after which the supernatant cytokine levels of IL-2, IL-4, IL-5 IL-10, IL-12p70, IL-13, IL-17, IFNγ, and TNF-α were measured by QUANSYS multiplex ELISA (9-plex) (Quansys Biosciences, Logan, Utah) following the manufacturer's recommendations.

Table 3 shows various cytokine production prior to challenge.

TABLE 3
Cytokine Production Prior to Challenge.

| Cytokine | | pQE30-null + | | | NirB_SspH1 + | rCatB + |
|---|---|---|---|---|---|---|
| (pg/mL) | PBS | rCatB | rCatB | NirB_SspH1 | rCatB | NirB_SspH1 |
| IL-2 | 424.5 ± 57.9 | 190.8 ± 62.3 | 426.9 ± 149.7 | 174.1 ± 23.5 | 324.6 ± 52.7 | 174.8 ± 62.0 |
| IL-4 | 20.6 ± 2.2 | 27.5 ± 6.4 | 18.0 ± 3.2 | 35.4 ± 7.6 | 22.8 ± 3.6 | 10.3 ± 1.7 |
| IL-10 | 10.2 ± 0.9 | 23.2 ± 4.3 | 29.7 ± 5.9 | 21.6 ± 2.4 | 21.9 ± 1.5 | 16.0 ± 3.1 |
| IL-12p70 | 34.5 ± 12.1 | 21.5 ± 5.8 | 16.5 ± 0.8 | 15.8 ± 0^{#} | 16.2 ± 0.4 | 15.8 ± 0^{#} |
| IL-13 | 23.0 ± 7.1 | 22.9 ± 8.7 | 75.1 ± 17.4 | 16.9 ± 6.1 | 68.8 ± 33.4 | 13.1 ± 2.4 |
| IL-17 | 19.4 ± 5.3 | 14.1 ± 0^{#} | 25.3 ± 11.0 | 14.1 ± 0^{#} | 14.5 ± 0.4 | 14.3 ± 0.2 |
| TNFα | 26.7 ± 6.2 | 36.1 ± 6.8 | 24.0 ± 5.9 | 17.1 ± 3.0 | 26.1 ± 3.8 | 32.0 ± 5.3 |

Supernatant levels of different cytokines after stimulating splenocytes with rCatB for 72 hours were measured by QUANSYS multiplex ELISA. These results represent 5-7 animals per group. Results are expressed as the mean+the standard error of the mean.

#Values were below the limit of detection.

Schistosoma mansoni Challenge

Biomphalaria glabrata snails infected with the S. mansoni Puerto Rican strain were obtained from the Schistosomiasis Resource Center of the Biomedical Research Institute (Rockville, Md.) through NIH-NIAID Contract HHSN272201700014I for distribution through BEI Resources. Mice were challenged three weeks after the second immunization (week 6) with 150 cercariae by tail exposure and were sacrificed seven weeks post-challenge as previously described [32]. Briefly, adult worms were counted after perfusion of the hepatic portal system and manual removal from the mesenteric veins. The livers and intestines were harvested from each mouse, weighed and digested in 4% potassium hydroxide overnight at 37° C. The next day, the number of eggs per gram of tissue was recorded by microscopy. A small portion of each liver was placed in 10% buffered formalin phosphate (Fisher Scientific) and processed for histopathology to assess mean granuloma size and egg morphology (H&E staining). Granuloma area was measured using Zen Blue software (version 2.5.75.0; Zeiss) as previously reported [33, 34]. Briefly, working at 400× magnification, the screen stylus was used to trace the perimeter of 6-8 granulomas with a clearly visible egg per mouse which the software converted into an area. Mean areas were presented as ×10³ μm²±SEM. Eggs were classified as abnormal if obvious shrinkage had occurred, if internal structure was lost or if the perimeter of the egg was crenelated and are reported as a percent of the total eggs counted (±SEM).

TABLE 4
Granuloma size and egg morphology

| | Granuloma size | Abnormal egg |
|---|---|---|
| | (× 10³ μm²) | morphology |
| Group | ± SEM | (%) ± SEM |
| PBS | 62.2 ± 6.1 | 0 |
| pQE-30 null + rCatB | 52.0 ± 6.9 | 18.9 ± 3.9 |
| rCatB | 52.8 ± 10.4 | 12.6 ± 5.1 |
| SspH1_SspH1 | 55.0 ± 8.5 | 25.0 ± 6.2 |
| SspH1_SspH1 + rCatB | 47.3 ± 4.4 | 30.5 ± 7.7* |
| rCatB + SspH1_SspH1 | 49.8 ± 14.3 | 28.6 ± 6.8* |
| nirB_SspH1 | 32.9 ± 2.0** | 75.9 ± 7.6**** |
| nirB_SspH1 + rCatB | 34.7 ± 3.4** | 79.4 ± 4.2**** |
| rCatB + nirB_SspH1 | 39.2 ± 3.7* | 71.9 ± 6.0**** |

Liver granuloma area (×10³ μm²) and egg morphology (ie: loss of internal structures, shrinkage, crenelated periphery) were assessed. Each group consists of either a saline control, EV→IM, PO→PO, IM→IM, PO→IM, and IM→PO for the nirB_SspH1 and/or the SspH1_SspH1 construct. SEM represents the standard error of the mean. (*P<0.05, **P<0.01, ****P<0.0001 compared to the PBS group)

Statistical Analysis

Statistical analysis was performed using GraphPad Prism 6 software (La Jolla, Calif.). In each experiment, reductions in worm and egg burden were expressed relative to the saline control group numbers. Results are represented from two separate experiments. Data were analyzed by one-way ANOVA and multiple comparisons were corrected using Tukey's multiple comparison procedure. P values less than 0.05 were considered significant.

Results

In Vitro Expression and Secretion of CatB by Transformed YS1646 Strains

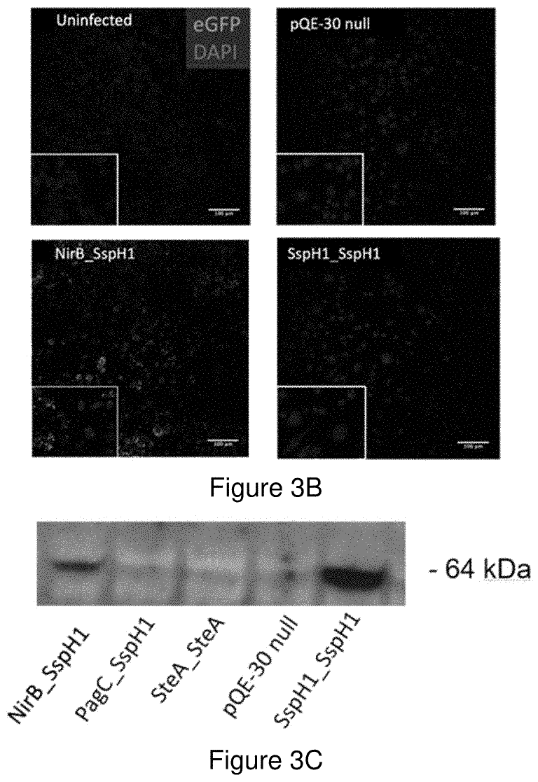

Thirteen expression cassettes were built and the sequences were verified (McGill University Genome Quebec Innovation Centre) (Table 1). The promoter/T3SS pairs were inserted in-frame with either S. mansoni CatB or eGFP. In monomicrobial culture, CatB expression was effectively driven by the nirB_SspH1, SspH1_SspH1 and SteA_SteA plasmids (FIG. 3A) with the greatest production from the nirB promoter in low oxygen conditions as previously reported [29]. Secreted CatB was detectable in the monomicrobial culture supernatants only with YS1646 bearing the SspH1_SspH1 construct (FIG. 3A). In infected RAW 264.7 cells, all of the constructs produced detectable eGFP by immunofluorescence (FIG. 3B) but only the YS1646 bearing the nirB_SspH1 and SspH1_SspH1 constructs produced CatB detectable by immunoblot (FIG. 3C). These constructs also led to the greatest eGFP expression in the RAW 264.7 cells and so were selected for in vivo testing.

FIGS. 3A-3C show expression of recombinant Cathepsin B. FIG. 3A: The plasmids nirB_SspH1, SspH1_SspH1 and SteA_SteA were transformed into Salmonella strain YS1646. Whole bacteria lysates and monomicrobial culture supernatants were examined for the presence of CatB by western blot. FIG. 3B: The mouse macrophage cell line RAW 264.7 cells were infected with transformed YS1646 strains expressing eGFP as a marker for the capacity of promoter-TSSS pairs to support expression of a foreign protein. DAPI nuclear stain is represented in blue and eGFP is shown in green. Scale at 100 μm. FIG. 3C: Mouse macrophage cells line RAW 264.7 cells were infected with selected plasmids from Table 1 and the presence of CatB protein was determined by western blotting.

Antibody Response to YS1646-Vectored Vaccination

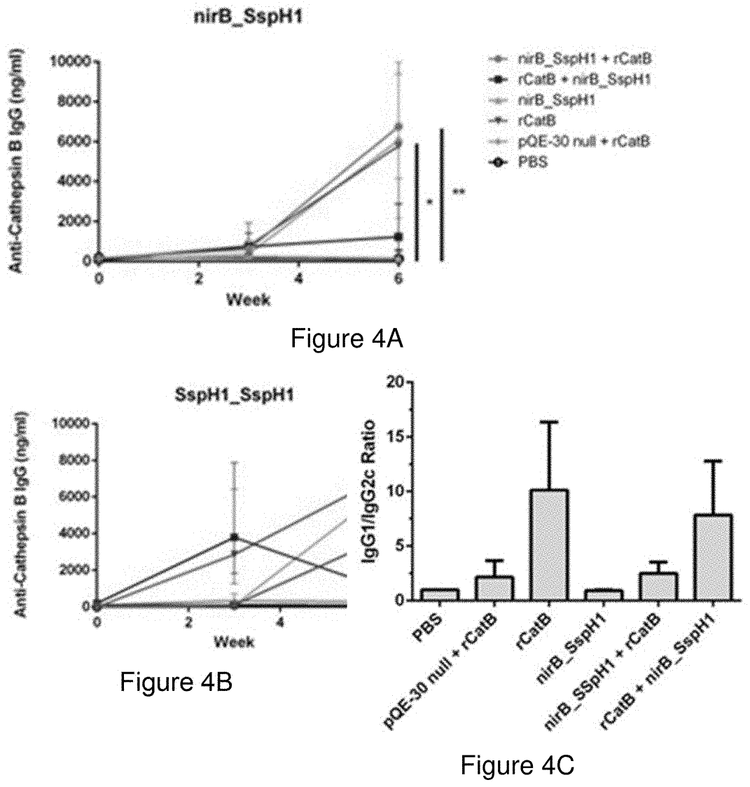

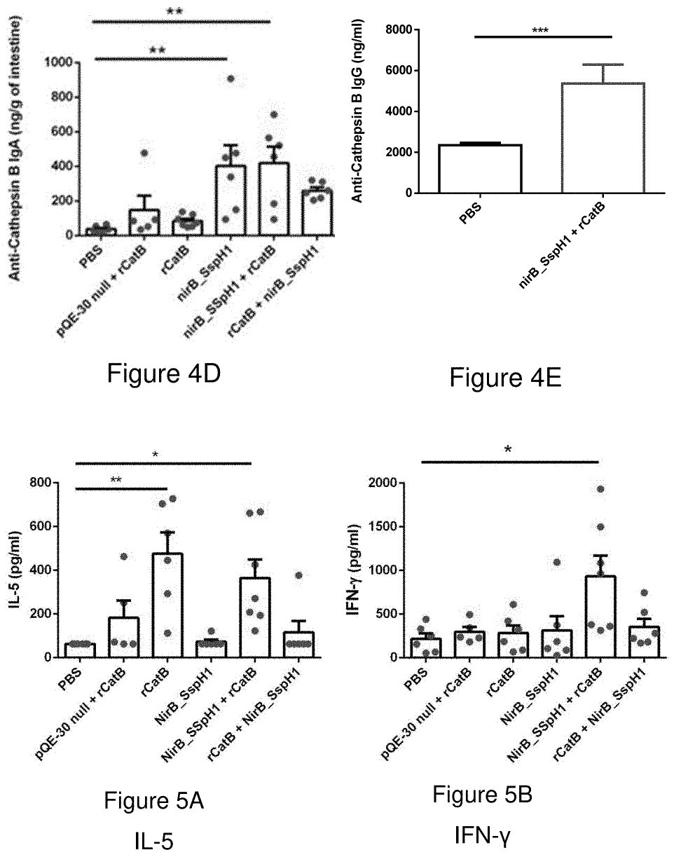

None of the groups had detectable anti-CatB IgG antibodies at baseline and the saline control mice remained negative after vaccination. Mice in the PO→PO group also had very low serum CatB-specific IgG antibody levels even after the second vaccination (395.7±48.9: FIG. 4A). In contrast, all animals that had received at least 1 dose of rCatB IM had significantly higher IgG titers at 6 weeks (ie: 3 weeks after the second immunization) (FIG. 4A). Mice that received nirB_SspH1 PO followed by an IM boost had the highest titers (6766±2128 ng/mL, P<0.01 vs. control) but these titers were not significantly different from groups that had received either one (EV→IM) or two doses of rCatB (IM→IM) (5898±1951 ng/mL and 6077±4460 ng/mL respectively, both P<0.05 vs. control). IgG antibody titers were generally lower in all groups that received the YS1646 strain bearing the SspH1_SspH1 construct (range 333.5-3495 ng/mL; P<0.05, P<0.01, P<0.001 vs control: FIG. 4B). Because the SspH1_SspH1 construct will not be carried forward into more advanced studies, we did not measure the IgG subtypes or the intestinal IgA levels for these experimental groups.

Control mice had no detectable anti-CatB antibodies and were arbitrarily assigned an IgG1/IgG2c ratio of 1. The PO→PO mice had a ratio of 0.9 (FIG. 4C). The EV→IM and the PO→IM groups had IgG1/IgG2c ratios of 2.2 and 2.5 respectively while the highest ratios were seen in the IM→IM and IM→PO groups (10.2 and 7.8 respectively).

Intestinal IgA levels in the saline, EV→IM, and IM→IM groups were all low (range 37.0-148.0 ng/g of tissue: FIG. 4D). Although the data are variable, groups that received at least one dose of nirB_SspH1 YS1646 PO had increased IgA levels compared to the control group that reached statistical significance in the PO→PO group (402.7±119.7 ng/g) and the PO→IM group (ie: nirB_SspH1 PO then rCatB IM: 419.6±95.3 ng/g, both P<0.01). The IM→PO group also had higher intestinal IgA titers than controls, but this increase did not reach statistical significance (259.8±19.4 ng/g).

FIGS. 4A-4E show production of Sm-Cathepsin B specific antibodies prior to challenge. Serum anti-CatB IgG was measured by ELISA at weeks 0, 3 and 6 for groups that received the nirB_SspH1 construct (A) or the SspH1_SspH1 construct (B). Each group consists of either a saline control, EV→IM, PO→PO, IM→IM, PO→IM, and IM→PO for the nirB_SspH1 and/or the SspH1_SspH1 construct. These results represent between 8-16 animals/group from 2 independent experiments and are reported as the geometric mean with 95% confidence intervals. Significance bars for A and B are to the right of each graph. C) Serum anti-CatB IgG1 and IgG2c were measured by endpoint-dilution ELISA and expressed as the ratio of IgG1/IgG2c. D) Intestinal anti-CatB IgA in intestinal tissue was measured by ELISA and is reported as mean±standard error of the mean ng/gram. These results represent 5-7 animals per group. (*P<0.05, **P<0.01, ** *P<0.001 compared to the PBS group). FIG. 4E shows intestinal anti-CatB IgG measured by ELISA and is reported as mean±standard error of the mean ng/gram. Statistical test: One-way ANOVA, Tukeys multiple comparison (P<0.001).

Cytokine Production in Response to YS1646-Vectored Vaccination

There was only modest evidence of CatB-specific cytokine production by antigen re-stimulated splenocytes immediately prior to challenge (4 weeks after the second dose). There were no significant differences in the levels of IL-2, IL-4, IL-10, IL-12p70, IL-13, IL-17 or TNF-α between vaccinated and control groups (Table 3). Compared to the control group, the levels of IL-5 in splenocyte supernatants were significantly higher in mice that received two doses of rCatB (IM→IM) (475.5±98.5 pg/mL, P<0.01) and the nirB_SspH1 PO→IM group (364.4±85.2 pg/mL, P<0.05) whereas the control group was below the limit of detection at 63.1 pg/mL (FIG. 5A). Only the PO→IM group had clear evidence of CatB-specific production of IFNγ in response to vaccination (933±237 pg/mL vs. control 216.4±62.5 pg/mL, P<0.05) (FIG. 5B). FIGS. 5A and 5B show cytokine production prior to challenge. Supernatant IL-5 (A) and IFN-γ (B) levels after stimulating splenocytes with rCatB for 72 hours were measured by QUANSYS multiplex ELISA. Each group consists of either a saline control, EV→IM, PO→PO, IM→IM, PO→IM, and IM→PO for the nirB_SspH1 and/or the SspH1_SspH1 construct. These results represent 5-7 animals per group. Results are expressed as the mean±the standard error of the mean. (*P<0.05, **P<0.01 compared to the PBS group)

Protection from S. mansoni Challenge from YS1646-Vectored Vaccination

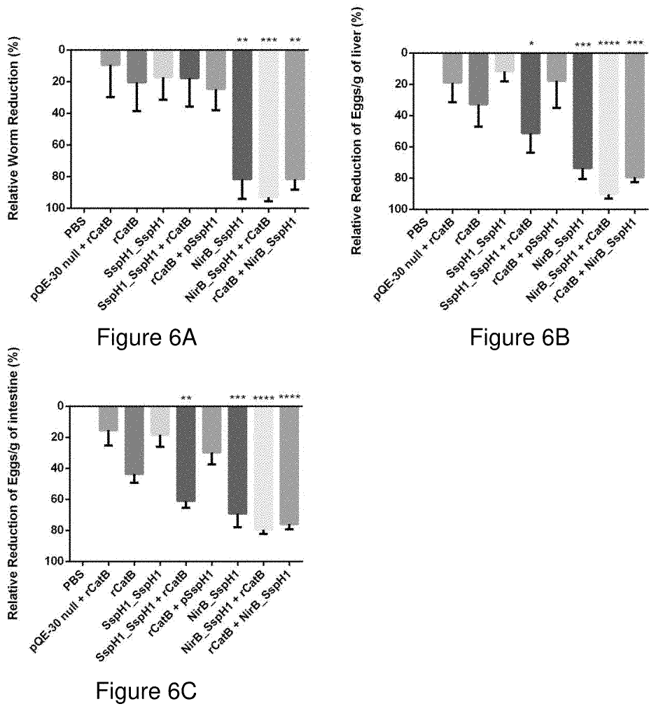

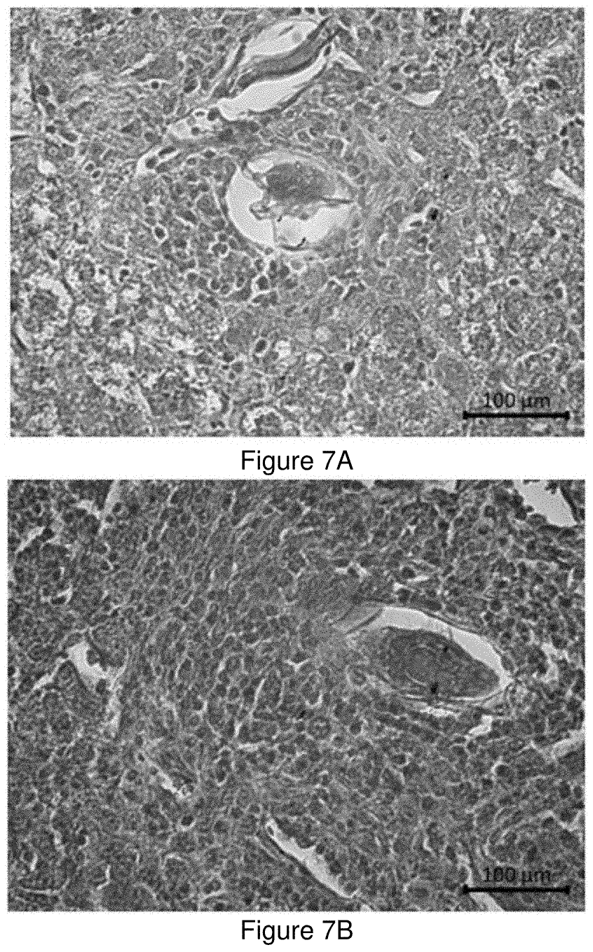

At 7 weeks after infection, the mean worm burden in the saline-vaccinated control group was 25.2±4.3 and all changes in parasitologic and immunologic outcomes are expressed in reference to this control group. Relatively small reductions in worm burden were observed in the EV→IM (9.4%) and IM→IM groups (20.5%) across all studies. Overall, protection was better with nirB_SspH1_CatB schedules compared to SspH1_SspH1_CatB schedules. In the SspH1_SspH1 animals, reductions in worm numbers were similar to the IM→IM group: 17.2% with oral vaccination alone (PO→PO) and only 17.8% and 24.7% in the PO→IM and IM→PO groups respectively. In contrast, the PO→PO group vaccinated with the nirB_SspH1 YS1646 strain had an 81.7% (P<0.01) reduction in worm numbers and multi-modality vaccination with this strain achieved 93.1% (P<0.001) and 81.7% (P<0.01) reductions in the PO→IM and IM→PO groups respectively. (FIG. 6A).

Overall, the reductions in hepatic and intestinal egg burden followed a similar pattern to the vaccine-induced changes in worm numbers. The hepatic and intestinal egg burden in the saline-vaccinated control mice ranged from 1,994-13,224 eggs/g and 6,548-24,401 eggs/g respectively. Reductions in hepatic eggs in the EV→IM and IM→IM groups were modest at 18.9% and 32.7% respectively. Reductions in intestinal eggs followed a similar trend: 15.4% and 43.6% respectively. In the groups that received the SspH1_SspH1 YS1646 strain, PO→PO immunization did not perform any better with 11.6% and 18.3% reductions in hepatic and intestinal egg numbers respectively. Somewhat greater reductions in hepatic and intestinal egg burden were seen in the PO→IM (51.3% and 60.9% respectively) and IM→PO groups (17.7% and 29.8% respectively). These apparent differences in egg burden between the two multi-modality groups did not parallel the reductions in worm numbers or the systemic anti-CatB IgG levels. Groups that received the nirB_SspH1 strain had more consistent and greater reductions in egg burden: the PO→PO group had 73.6% and 69.2% reductions in hepatic and intestinal egg numbers respectively (both P<0.001). The greatest impact on hepatic and intestinal egg burden was seen in the nirB_SspH1 multi-modality groups: 90.3% (P<0.0001) and 79.5% (P<0.0001) respectively in the PO→IM group and 79.4% (P<0.001) and 75.9% (P<0.0001) respectively in the IM→PO group (FIGS. 6B and 6C).

As shown in FIGS. 6A-6C, the reduction in worm counts (A) as well as the reduction in egg load per gram of liver (B) or intestine (C) are represented for mice in the each group consisting of either a saline control, EV→IM, PO→PO, IM→IM, PO→IM, and IM→PO for the nirB_SspH1 and/or the SspH1_SspH1 construct. Worm and egg burdens were determined 7 weeks after cercarial challenge. These results represent between 8-16 animals/group from 2 independent experiments. (*P<0.05, **P<0.01, ***P<0.001, ****P<0.0001 compared to the PBS group)

Hepatic granulomas were large and well-formed in the PBS-treated control mice (62.2±6.1×10³ μm²) and essentially all of the eggs in these granulomas had a normal appearance. The EV→IM and IM→IM groups had slightly smaller granulomas (52.0±6.9×10³ μm² and 52.8±10.4×10³ μm² respectively) with modest numbers of abnormal-appearing eggs (ie: loss of internal structure, crenellated edge) (Table 4) but these differences did not reach statistical significance. Groups that received the SspH1_SspH1 strain had granuloma sizes ranging from 47.3-55.0×10³ μm² with 30.5% of the eggs appearing abnormal in the PO→IM and 28.6% IM→PO groups (both P<0.05). In the groups that received the nirB_SspH1 strain, both the purely oral (PO→PO) and multi-modality strategies (PO→IM and IM→PO) resulted in even smaller granulomas (32.9±2.0 μm², 34.7±3.4×10³ μm² and 39.2±3.7×10³ μm²: P<0.01, P<0.01 and P<0.05 respectively). The large majority of the eggs in these granulomas had disrupted morphology (75.9±7.6%, 79.4±4.2% and 71.9±6.0% respectively: all P<0.0001). Overall, the greatest and most consistent reductions in both adult worm numbers and egg burdens in hepatic and intestinal tissues were seen in the animals that received oral dosing with the YS1646 bearing the nirB_SspH1_CatB construct followed 3 weeks later by IM rCatB.

### DISCUSSION

S. mansoni vaccine candidate capable of providing >40% protection [9]. This initiative targeted reduced worm numbers as well as reductions in egg burden in both the liver and the intestinal tissues. S. mansoni female worms can produce hundreds of eggs per day [33]. While the majority are excreted in the feces, some are trapped in host tissues where they cause most of the pathology associated with chronic infection [34]. Eggs trapped in the liver typically induce a vigorous granulomatous response that can lead to fibrosis, cirrhosis and death while egg-induced granulomas in the intestine cause local lesions that contribute to colonic polyp formation [35].

The protective efficacy of CatB-based vaccines delivered IM with adjuvants has been previously described. Using CpG dinucleotides to promote a Th1-type response, vaccination resulted in a 59% reduction in worm burden after challenge with 56% and 54% decreases in hepatic and intestinal egg burden respectively compared to adjuvant-alone control animals [12]. Parasitologic outcomes were slightly better in the same challenge model when the oil-in-water adjuvant Montanide ISA 720 VG was used to improve the antibody response: 56-62% reductions in worm numbers and the egg burden in tissues [13]. These results were well above the 40% threshold suggested by the TDR/WHO and provided proof-of-concept for CatB as a promising target antigen. Based on this success, we expanded our vaccine discovery program to explore alternate strategies and potentially more powerful delivery systems. enterica species replicate in a membrane-bound host cell compartment or vacuole [36], foreign protein antigens can be efficiently exported from the vacuole into the cytoplasm using the organism's T3SS. Like all Salmonella enterica species, YS1646 has two distinct T3SS located in Salmonella pathogenicity islands 1 and 2 (SPI-I and SPI-II) [37] that are active at different phases of infection [38]. The SPI-I T3SS translocates proteins upon first contact of the bacterium with epithelium cells through to the stage of early cell invasion while SPI-II expression is induced once the bacterium has been phagocytosed [39]. These T3SS have been used by many groups to deliver heterologous antigens in Salmonella-based vaccine development programs [22, 40].

The protective efficacy of CatB delivered by the attenuated strain YS1646 of Salmonella enterica serovar Typhimurium in a heterologous prime-boost vaccination regimen is described. Compared to infected controls, vaccination with CatB IM followed by YS1646 bearing the nirB_SspH1 strain resulted in an 93.1% reduction in worm numbers and 90.3% and 79.5% reductions in hepatic and intestinal egg burdens respectively compared to the control group. These results not only surpass the WHO's criterion for an effective S. mansoni vaccine by a considerable margin, they are a marked improvement on our own work using CatB delivered IM with adjuvants and are among the best results ever reported in similar murine models [12, 13]. For example, in the pre-clinical development of two candidate vaccines that subsequently entered clinical trials [43, 44], IM administration of the fatty acid binding protein Sm-14 with the adjuvant GLA-SE led to a 67% reduction in worm burden in mice [10] while IM vaccination with the tegumental protein TSP-2 with either Freund's adjuvant or alum/CpG reduced worm numbers by 57% and 25% and hepatic egg burden by 64% and 27% respectively [45, 46]. Another vaccine candidate targeting the tegumental protein Sm-p80 that is advancing towards clinical testing achieved 70 and 75% reductions in adult worm numbers and hepatic egg burden respectively when given IM with the oligodeoxynucleotide (ODN) adjuvant 10104 [47]. It is noteworthy that these other vaccine candidates were all administered IM, a route that typically results primarily in systemic immunity. Although there are reports of vaccines delivered IM that can induce some level of mucosal immunity [48], particularly with the use of adjuvants, intramuscular injection is less likely to elicit a local, mucosal response than the multimodality approach taken in our studies.

It is noteworthy that these other vaccine candidates were all administered IM. Although this route would be expected to generate high systemic antibody titers, particularly with the use of adjuvants, it is unlikely that any would elicit a local, mucosal response like the multimodality approach taken in our studies.

To what extent the surprising reductions in worm and egg burdens that we observed with the YS1646 can be attributed to the systemic or the local antibody response is currently unknown although it is likely that both contributed to the success of the combined schedules (ie: IM→PO and PO→IM). Oral administration of Salmonella-vectored vaccines clearly leads to higher mucosal IgA responses than IM dosing [49] and the protective potential of IgA antibodies has been demonstrated in schistosomiasis [50]. The migrating schistosomulae likely interact with the MALT during their week-long passage through the lungs. It is therefore possible that IgA produced by the respiratory mucosa interferes with parasite development at this stage in its lifecycle. The importance of the local response is strongly suggested by the fact that PO dosing alone with YS1646 bearing the nirB_SspH1_CatB construct still provided substantial protection (81.7% and 73.6%/69.2% for worms and hepatic/intestinal eggs) despite the almost complete absence of a detectable systemic response (FIG. 4A). Indeed, IgA titers were readily detectable in the intestinal tissues of mice receiving the nirB_SspH1 YS1646 vaccine PO→PO and in mice the received PO→IM dosing (402.7 ng/g and 419.6 ng/g respectively) (FIG. 4D). On the other hand, the importance of IgG antibodies in the protection against schistosomiasis has been reported by many groups [51, 52]. Administered IM, rCatB alone consistently elicited high systemic antibody responses and provided a modest level of protection without any measurable mucosal response. Chen and colleagues have also used YS1646 as a vector to test single- and multi-modality approaches for a bivalent vaccine candidate (Sj23LHD-GST) targeting S. japonicum in a similar murine model [29]. Although some authors have promoted so-called ‘prime-pull’ strategies to optimize mucosal responses (ie: ‘prime’ in the periphery then ‘pull’ to the target mucosa) [53], it is interesting that both the Chen group and our own findings suggest that PO→IM dosing may be the optimal strategy. In the S. japonicum model targeting the long hydrophobic domain of the surface exposed membrane protein Sj23LHD and a host-parasite interface enzyme (glutathione S-transferase or GST), the PO→IM vaccination schedule led to important reductions in both worm numbers (51.4%) and liver egg burden (62.6%) [29].

In addition to the substantial overall reductions in worm numbers and egg burden in our animals that received multimodality vaccination, there were additional suggestions of benefit in terms of both hepatic granuloma size and possible reduced egg fitness (Table 2). The size of liver granulomas is determined largely by a Th2-deviated immune response driven by soluble egg antigens (SEA) [54]. Prior work with CatB vaccination suggests that IM delivery of this antigen alone tends to elicit a Th2-biased response that can be shifted towards a more balanced Th1/Th2 response by CpG or Montanide [12, 13, 55]. The reduction in the anti-CatB IgG1/IgG2c ratio between the IM→IM only and multimodality groups (IM→PO, PO→IM) supports the possibility that combined recombinant CatB with YS1646 bearing CatB can induce a more ‘balanced’ pattern of immunity to this antigen and, at least in a limited sense, that the YS1646 is acting as a Th1-type adjuvant (FIG. 4C). Although no adjuvants were included in the current study, the YS1646 vector might reasonably be considered ‘auto-adjuvanted’ by the presence of LPS, even in an attenuated form, and flagellin which can act as TLR-4 and TLR-5 agonists respectively. It was still surprising however, that the average hepatic granuloma size was significantly smaller in our multi-modality groups than in the IM alone group since no CatB is produced by the eggs (Table 2). This observation raises the interesting possibility that the YS1646-based vaccination protocol may be able to influence the overall pattern of immunity to S. mansoni and/or reduce the fitness of the eggs produced (as suggested by the abnormal egg morphology observed). Such effects could significantly extend the value of the combined PO→IM vaccination strategy, i.e.: more durable impact, reduced transmission, etc. Furthermore, prior work with IM vaccination with CatB alone revealed a Th2-type pattern of cytokine response in splenocytes (eg: IL-4, IL-5, and IL-13) [55]. In the current work we observed increases in both IFNγ and IL-5 in the multimodality PO→IM group (FIG. 5), suggesting that YS1646 vaccination can induce more balanced Th1-Th2 immune response. Finally, this study did not consider the possible role of other immune mechanisms in controlling S. mansoni infection after YS1646 infection and we have previously shown that CD4⁺ T cells and anti-schistosomula antibody-dependent cellular cytotoxicity (ADCC) contribute to protection after CatB immunization (± adjuvants) [56]. Studies are underway to examine these possibilities with the multi-modality YS1646-based vaccination protocols. It is also intriguing that the apparent efficacy of either one or two IM doses of rCatB differed considerably between the EV→IM and IM→IM groups with the latter schedule eliciting significantly greater protection for all parasitologic outcomes despite the fact that these groups had similar levels of serum anti-CatB IgG at the time of challenge (FIG. 4). Future studies will address whether or not there are qualitative differences in the antibodies induced (ie: avidity, isotype, competence to mediate ADCC) and/or differences in other immune effectors (ie: CD4⁺ or CD8⁻ T cells).

Immune protection may be relatively narrow when only a single schistosome antigen is targeted. In the long term, this limitation could be easily overcome by adding one or more of the many S. mansoni target antigens that have shown promise in pre-clinical and/or clinical development (e.g., GST, Sm23, Sm-p80, etc.) to generate a ‘cocktail’ vaccine. In this context, an attenuated Salmonella vector like YS1646 might be ideal because of its high ‘carrying capacity’ for foreign genes [57]. Second, our current findings are based on plasmid-mediated expression and pQE30 contains a mobile ampicillin resistance gene that would obviously be inappropriate for use in humans [58]. Although chromosomal integration of our nirB_SspH1_CatB gene is an obvious mitigation strategy, expression of the CatB antigen from a single or even multiple copies of an integrated gene would likely be lower than plasmid-driven expression. Finally, the degree to which a vaccination schedule based on the YS1646 vector would be accepted by regulators is currently unknown. Attenuated Salmonellae have a good safety track-record in vaccination: e.g., the Ty21 a S. typhi vaccine and a wide range of candidate vaccines [57] despite their ability to colonize/persist for short periods of time [59]. Although the total clinical exposure to YS1646 to date is limited (25 subjects with advanced cancer in a phase 1 anti-cancer trial), the available data are reassuring since up to 3×10⁸ bacteria could be delivered intravenously in these vulnerable subjects without causing serious side effects [16]. Finally, these experiments were designed to test the simplest prime-boost strategies based on the YS1646 vaccine so no adjuvants were used with the recombinant protein dose. Experiments are on-going to determine whether or not the inclusion of an adjuvant with either the prime or boost dose of the recombinant protein can further enhance protection.

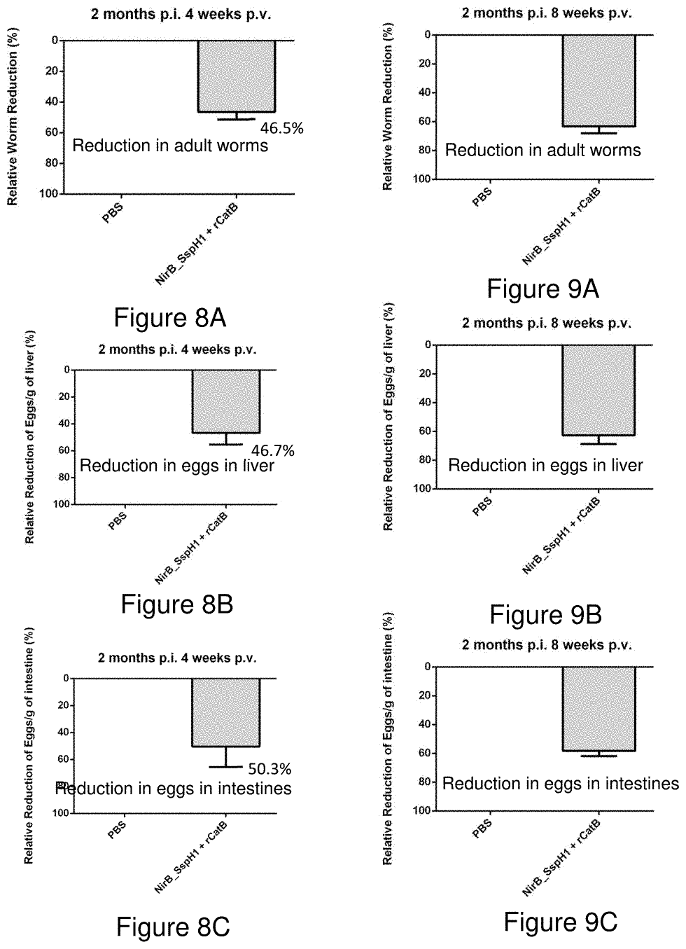

FIGS. 8A-8C show reductions in adult worms (FIG. 8A), eggs in liver (FIG. 8B), and eggs in intestines (FIG. 8C), two months after infection and four weeks after vaccination (two replicate experiments with ˜12 animals in each group).

FIGS. 9A-9C show reductions in adult worms (FIG. 9A), eggs in liver (FIG. 9B), and eggs in intestines (FIG. 9C), two months after infection and eight weeks after vaccination (two replicate experiments with ˜12 animals in each group).

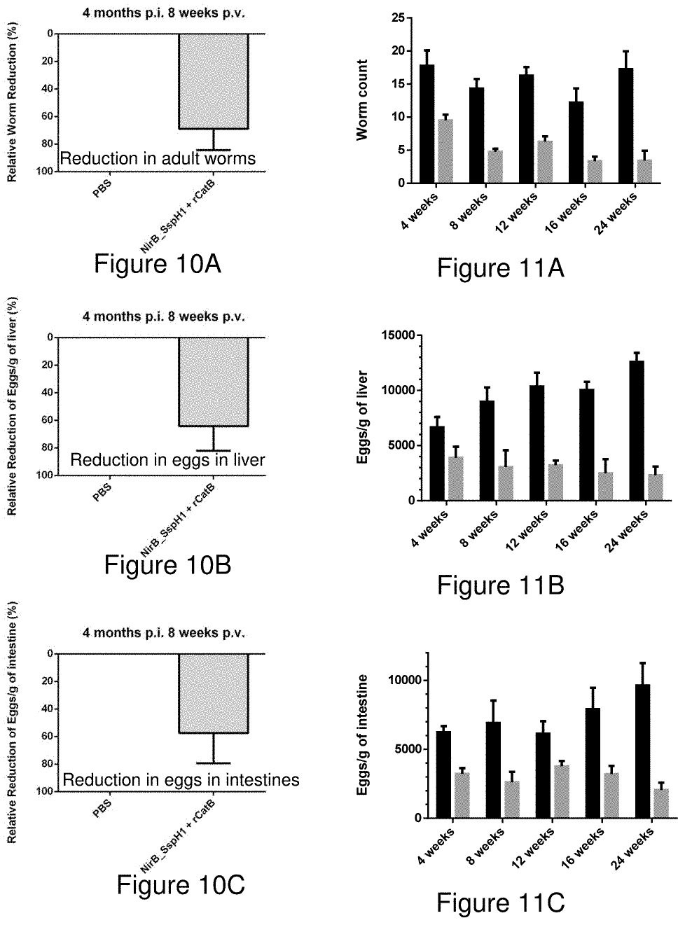

FIGS. 10A-10C show reductions in adult worms (FIG. 10A), eggs in liver (FIG. 10B), and eggs in intestines (FIG. 10C), four months after infection and eight weeks after vaccination (one experiment with ˜8 animals in each group).

In the experiment investigating the therapeutic use of nirB_SspH1_CatB vaccine, in a 5-day vaccination schedule, corresponding to FIGS. 8A-8C, 9A-9C, and 10A-10C, there are progressive decreases in parasitologic outcomes between 4 and 8 weeks after vaccination. Since these outcomes are vs. PBS controls, it cannot be determined if changes due to continued increases in PBS controls or further decreases in vaccinated animals (or both).

FIGS. 11A-11C show reductions in adult worms (FIG. 11A), eggs per gram in liver (FIG. 11B), eggs in intestine (FIG. 11C), over six months after infection.

TABLE 5
Therapeutic vaccine

| | 2 mo p.i. | 2 mo p.i. | 4 mo p.i. |
|---|---|---|---|
| Readout | 4 wks p.v. | 8 wks p.v. | 8 wks p.v |
| Relative worm | 46.5 | 63.2 | 69.0 |
| reduction | | | |
| Relative hapatic | 46.7 | 62.7 | 64.3 |
| egg reduction | | | |
| Relative intestinal | 50.3 | 58.2 | 57.4 |
| egg reduction | | | |

In summary, this work demonstrates that a YS1646-based, multimodality, prime-boost immunization schedule can provide nearly complete protection against S. mansoni in a well-established murine model. The protection achieved against a range of parasitologic outcomes was the highest reported to date for any vaccine. Therefore, the results are reasonably predictive of human response to the vaccine, subject to routine optimizations and known considerations.

What is claimed is:

1. A pharmaceutically acceptable vaccine kit, comprising:
an attenuated recombinant bacterium adapted to express at least one parasitic worm antigen based on a recombinant construct within the attenuated recombinant bacterium; and
a sterile injectable formulation comprising the at least one parasitic worm antigen.

2. The pharmaceutically acceptable vaccine kit according to claim 1, wherein the at least one parasitic worm antigen is secreted from the Salmonella bacteria by a Salmonella Type 3 secretion system.

3. The pharmaceutically acceptable vaccine kit according to claim 1, wherein the at least one parasitic worm antigen is catB.

4. The pharmaceutically acceptable vaccine kit according to claim 1, wherein the at least one parasitic worm antigen is expressed in a fusion peptide with a secretory signal selected from the group consisting of one or more of SopE2, SseJ, SptP, SspH1, SspH2, SteA, and SteB.

5. The pharmaceutically acceptable vaccine kit according to claim 1, wherein the transcription of the at least one parasitic worm antigen is under control of at least one promoter selected from the group consisting of one or more of SopE2, SseJ, SptP, SspH1, SspH2, SteA, SteB, pagC, lac, nirB, and pagC.

6. The pharmaceutically acceptable vaccine kit according to claim 1, wherein the at least one parasitic worm antigen is produced based on a chromosomally integrated genetically engineered construct.

7. The pharmaceutically acceptable vaccine kit according to claim 1, wherein the at least one parasitic worm antigen is produced based on a plasmid genetically engineered construct.

8. The pharmaceutically acceptable vaccine kit according to claim 1, wherein the at least one parasitic worm antigen is produced based on a genetically engineered construct comprising a promoter portion, a secretion signal portion, and a parasitic worm antigen portion.

9. The pharmaceutically acceptable vaccine kit according to claim 8, wherein the promoter portion and the secretion signal portion are separated by a first restriction endonuclease cleavage site.

10. The pharmaceutically acceptable vaccine kit according to claim 8, wherein the secretion signal portion and the parasitic worm antigen portion are separated by a second restriction endonuclease cleavage site.

11. A recombinant attenuated bacterium adapted for growth in a mammal, expressing at least one antigen corresponding to a schistosome antigen, adapted to induce a vaccine response to a schistosome after oral administration to the mammal.

12. The recombinant attenuated bacterium according to claim 11, in combination with an injectable form of the at least one antigen corresponding to the schistosome antigen.

13. A method of immunizing a human against a parasitic worm, comprising:
orally administering a live attenuated recombinant bacterium adapted to colonize an enteric tissue of the human, expressing at least one antigen corresponding to a parasitic worm antigen; and
injecting a sterile injectable vaccine comprising the at least one antigen corresponding to a parasitic worm antigen.

14. The method according to claim 13, wherein the at least one antigen corresponding to the parasitic worm antigen comprises CatB.

15. The method according to claim 13, wherein said injecting the sterile injectable vaccine comprises intramuscularly injecting the sterile injectable vaccine.

16. The method according to claim 13, wherein the sterile injectable vaccine comprises an adjuvant.

17. The method according to claim 13, wherein said administering of the live attenuated recombinant bacterium and the sterile injectable vaccine are at different times according to a predetermined temporal administration protocol.

18. The method according to claim 13, wherein said administering of the live attenuated recombinant bacterium precedes the administering of the sterile injectable vaccine by at least 24 hours.

19. The method according to claim 13, wherein the live attenuated recombinant bacterium is Salmonella enterica.

20. The method according to claim 13, wherein the parasitic worm comprises S. mansoni.


SEQUENCE LISTING (28 sequences)

SEQ ID NO: 1 - DNA, 29 bp, Artificial Sequence
  SopE2 promoter and secretory signal forward primer
ccgctcgagtaaaaatgttcctcgataaa

SEQ ID NO: 2 - DNA, 20 bp, Artificial Sequence
  SopE2 promoter and secretory signal reverse primer
catggtagttctccttttag

SEQ ID NO: 3 - DNA, 27 bp, Artificial Sequence
  SptP promoter and secretory signal forward primer
cgcctcgagtttacgctgactcattgg

SEQ ID NO: 4 - DNA, 23 bp, Artificial Sequence
  SptP promoter and secretory signal reverse primer
catttttctctcctcatacttta

SEQ ID NO: 5 - DNA, 27 bp, Artificial Sequence
  SseJ promoter and secretory signal forward primer
cgcctcgagacataaaacactagcact

SEQ ID NO: 6 - DNA, 27 bp, Artificial Sequence
  SseJ promoter and secretory signal reverse primer
cgcctcgagacataaaacactagcact

SEQ ID NO: 7 - DNA, 26 bp, Artificial Sequence
  SspH1 promoter and secretory signal forward primer
cgcctcgagcgctatatcaccaaaac

SEQ ID NO: 8 - DNA, 29 bp, Artificial Sequence
  SspH1 promoter and secretory signal reverse primer
ctctgcggccgcggtaagacctgacgctc

SEQ ID NO: 9 - DNA, 24 bp, Artificial Sequence
  SspH2 promoter and secretory signal forward primer
cgcctcgaggtttgtgcgtcgtat

SEQ ID NO: 10 - DNA, 29 bp, Artificial Sequence
  SspH2 promoter and secretory signal reverse primer
ctctgcggccgcattcaggcaggcacgca

SEQ ID NO: 11 - DNA, 25 bp, Artificial Sequence
  SteA promoter and secretory signal forward primer
cgcctcgaggtttcgccgcatgttg

SEQ ID NO: 12 - DNA, 29 bp, Artificial Sequence
  SteA promoter and secretory signal reverse primer
ctctgcggccgcataattgtccaaatagt

SEQ ID NO: 13 - DNA, 25 bp, Artificial Sequence
  SteB promoter and secretory signal forward primer
cgcctcgagcgctccagcgcttcga

SEQ ID NO: 14 - DNA, 28 bp, Artificial Sequence
  SteB promoter and secretory signal reverse primer
ctctgcggccgctctgacattaccattt

SEQ ID NO: 15 - DNA, 78 bp, Artificial Sequence
  Lac promoter forward primer
cgcctcgagcattaggcaccccaggctttacactttatgcttccggctcgtatgttgtgtggaattgtgagcggataa

SEQ ID NO: 16 - DNA, 71 bp, Artificial Sequence
  Lac promoter reverse primer
gtggaattgtgagcggataacaatttcacacaggaaacagctatgaccatgactaacataacactatccac

SEQ ID NO: 17 - DNA, 28 bp, Artificial Sequence
  nirB promoter forward primer
cgcctcgagttgtggttaccggcccgat

SEQ ID NO: 18 - DNA, 31 bp, Artificial Sequence
  nirB promoter reverse primer
cgcgcggccgccggatctttactcgcattac

SEQ ID NO: 19 - DNA, 27 bp, Artificial Sequence
  pagC promoter forward primer
cgcctcgaggttaaccactcttaataa

SEQ ID NO: 20 - DNA, 20 bp, Artificial Sequence
  pagC promoter reverse primer
aacaactccttaatactact

SEQ ID NO: 21 - DNA, 53 bp, Artificial Sequence
  SopE2 Secretion Signal forward primer
ggcggtaatagaaaagaaatcgaggcaaaaatgactaacataacactatccac

SEQ ID NO: 22 - DNA, 28 bp, Artificial Sequence
  SopE2 Secretion Signal reverse primer
aagtcgcggccgccggatctttactcgc

SEQ ID NO: 23 - DNA, 58 bp, Artificial Sequence
  SspH1 Secretion Signal forward primer
ggcggtaatagaaaagaaatcgaggcaaaaatgtttaatatccgcaatacacaacctt

SEQ ID NO: 24 - DNA, 29 bp, Artificial Sequence
  SspH1 Secretion Signal reverse primer
ctctgcggccgcggtaagacctgacgctc

SEQ ID NO: 25 - DNA, 33 bp, Artificial Sequence
  Cathepsin B forward primer
cgcgcggccgcgcacatctctgttaaaaacgaa

SEQ ID NO: 26 - DNA, 33 bp, Artificial Sequence
  Cathepsin B reverse primer
agtcggcgcgccgtggtggtggtggtggtgcgg

SEQ ID NO: 27 - DNA, 27 bp, Artificial Sequence
  eGFP forward primer
cgcgcggccgcggtgagcaagggcgag

SEQ ID NO: 28 - DNA, 30 bp, Artificial Sequence
  eGFP reverse primer
agtcggcgcgccttacttgtacagctcgtc